SEQUENCE LISTING (12 sequences)

SEQ ID NO: 1 - RNA, 21 nt, H. sapiens
aagaugugcacaggugagcaa

SEQ ID NO: 2 - RNA, 21 nt, H. sapiens
aauagacauuggguucgccgu

SEQ ID NO: 3 - RNA, 21 nt, H. sapiens
aaggaugagaucuaugccaac

SEQ ID NO: 4 - protein, 15 aa, H. sapiens
RKHAQTFVALCATDV

SEQ ID NO: 5 - protein, 13 aa, H. sapiens
RKPRDLELPLSPS

SEQ ID NO: 6 - protein, 13 aa, H. sapiens
KKPKGLGLAPTLV

SEQ ID NO: 7 - protein, 13 aa, H. sapiens
KKPKGLEISAPPL

SEQ ID NO: 8 - protein, 14 aa, H. sapiens
KTEKVDLELFPSPD

SEQ ID NO: 9 - protein, 15 aa, H. sapiens
KRVKLDSVRVLRQIS

SEQ ID NO: 10 - protein, 14 aa, H. sapiens
KKKPTPIQLNPAPD

SEQ ID NO: 11 - protein, 15 aa, H. sapiens
RKTRHVNILLFMGYM

SEQ ID NO: 12 - protein, 13 aa, H. sapiens
KRRNPLSLPVEKI

(19) United States
(12) Patent Application Publication
(10) Pub. No.: US 2007/0026431 A1
(43) Pub. Date: Feb. 1, 2007

(54) Modulation of MAPK-mediated phosphorylation and/or FBXW8-mediated ubiquitinylation of cyclin D1 in modulation of cellular proliferation

(76) Inventors: Hiroshi Okabe, San Francisco, CA (US); Frank McCormick, San Francisco, CA (US); Osamu Tetsu, San Francisco, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/441,806

(22) Filed: May 26, 2006

Related U.S. Application Data
(60) Provisional application No. 60/685,057, filed on May 26, 2005.

(51) Int. Cl. C12Q 1/68; G01N 33/567; C12Q 1/48; C07H 21/04; C12P 21/06; C12N 9/10

(52) U.S. Cl. 435/6; 435/7.2; 435/69.1; 435/320.1; 435/325; 435/193; 435/15; 530/350; 536/23.2

(57) ABSTRACT

The invention features methods and compositions for screening for agents that modulate cellular proliferation, particularly in cells that have elevated cyclin D1 (e.g., cancerous cells), where the methods provide for detection of agents that modulate phosphorylation of cyclin D1 by MAPK and/or detection of agents that modulate ubiquitination of cyclin D1 by FBXW8. The invention also features methods of controlling cellular proliferation, and agents useful in such methods.

### CROSS-REFERENCE

This application claims the benefit of U.S. Provisional Application No. 60/685,057, filed May 26, 2005, which application is incorporated herein by reference in its entirety.

### BACKGROUND OF THE INVENTION

Cyclin D1 plays a key role in regulation of G1 progression of the cell cycle in cancer cells, and is over-expressed in various kinds of cancers. Increased expression of cyclin D1 is achieved by different mechanisms, including chromosomal rearrangements, gene amplifications, and mRNA stabilization. In addition, uterine leiomyosarcomas and endometrial cancers, and breast cancers are reported to have defects in the proteolysis of cyclin D1 protein. These observations have led to increased interest to understand the mechanism that confer increased levels of cyclin D1 in cells.

Transcriptional regulation of cyclin D1 has been extensively studied and is well understood. Various mitogenic signals that activate the Ras/Raf/MEK/ERK (MAPK) cascade, resulting in cyclin D1 synthesis and its assembly with CDK4/6 in the presence or absence of assembly factors p21 Cip1 or p27 Kip1. Cyclin D1 is also a major transcriptional target of the APC/β-catenin/TCF signaling pathway. Indeed, cancers having mutations either APC or β-catenin exhibit high levels of cyclin D1 expression. Further confirming the role of cyclin D1 in cancer progression, mice lacking cyclin D1 are resistant to colon cancers induced by hyperactivation of the β-catenin/TCF signaling pathway.

Other mechanisms for increasing cyclin D1 in a cell are not as well characterized. Cyclin D1 is polyubiquitinated and subsequently degraded through the 26S proteasome pathway, a process that requires phosphorylating cyclin D1 at threonine (Thr)-286, located near its C terminus (Diehl et al. (1997) Genes Dev 1:957-72; Diehl et al. (1997) Mol Cell Biol. 17: 7362-74). Phosphorylation of cyclin D1 promotes its nuclear-to-cytoplasmic redistribution, indicating a role of cyclin D1 phosphorylation in cell cycle regulation. The cyclin D1 mutant T286A is resistant to ubiquitination in vitro and in vivo and is a highly stable protein. However, the cell components responsible for cyclin D1 phosphorylation, and for degradation of phosphorylated cyclin D1, has been an area of continued research and conflicting reports.

For example, GSK3β has been implicated as having a role in cyclin D1 phosphorylation and stability (Diehl et al. (1998) Genes Dev 12: 3499-511; Alt et al. (2000) Genes Dev. 14: 3102-14), but this role was later questioned (Shao et al. (2000) J Biol Chem. 275: 22916-24; Guo et al. (2005) Oncogene 24: 2599-612). Others have reported that p38 SAPK2 is involved in proteasomal degradation of cyclin D1 following osmotic shock (Casanovas et al. (2000) J Biol Chem. 275:35091-7). Mitogen signals or Ras activates phosphatidylinositol-3-OH kinase (P13K) and protein kinase B (PKB/Akt) kinases, which in turn inhibit activity of GSK3β. Therefore Ras signals may contribute to stabilization of the cyclin D1 protein. However, in Rat-1 cells, Ras signals have the opposite effect on cyclin D1 protein: they promote cyclin D1 degradation but not stabilization, suggesting that in these cells cyclin D1 turnover is totally independent of GSK3β.

While ubiquitin-mediated degradation is a well understood process, the ubiquitin pathway enzymes that mediate degradation of Cyclin D1 were also not understood. In general, polyubiquitin-protein conjugates are formed by shuttling three components that participate in sequential ubiquitin transfer reactions: 1) E1, an activating enzyme, 2) E2/Ubc, a ubiquitin-conjugating enzyme, and 3) an E3 protein ligase, which specifically binds to the target protein substrate (Hershko et al. (1998) Annu Rev Biochem. 67:425-79). This process facilitates E2-dependent addition of a multiubiquitin chain to lysine residues in a substrate protein.

The multicomponent SCF E3 ubiquitin ligases regulate ubiquitination of substrates in a phosphorylation-dependent manner (Deshaies (1999) Annu Rev Cell Dev Biol 15: 435-67). The SCF E3 ubiquitin ligases are a highly diverse family of complexes named for its components, the S-phase kinase-associated protein 1 (SKP1), Cullin 1 (CUL1/Cdc53), F-box proteins, and RBX1/ROC1 (Cardozo et al. (2004) Nat Rev Mol Cell Biol. 5: 739-51; Jin et al. (2004) Genes Dev. 18: 2573-80). SKP1 is an adaptor subunit and selectively interacts with a scaffold protein CUL1 or CUL7 to promote the ubiquitination of targeted substrates. Association of CUL7 with SKP1 depends on FBXW8 (also known as Fbx29, FBXO29, or Fbw6; Jin et al., 2004) and forms a specific SCF-like complex (Dias et al. (2002) Proc Natl Acad Sci U S A. 99: 16601-6; Arai (2003) Proc Natl Acad Sci USA. 100: 9855-60). Currently it is thought that CUL1, and perhaps CUL7 as well, are covalently modified by NeddS, a ubiquitin-like molecule involving recruitment of the RTNG-containing protein RJBX1, which in turn recruits an E2 ubiquitin-conjugating enzyme to the SCF, and may also facilitate recruitment of the SCF-like E3 ubiquitin ligase complex (Lammer et al. (1998) Genes Dev. 12: 914-26; Osaka et al. (1998) Genes Dev. 12: 2263-8; Kawakami et al. (2001) EMBO J. 20: 4003-12).

There is a need for compounds that modulate cellular proliferation, particularly compounds that inhibitor cellular proliferation of cancer cells. Identification of the cellular proteins involved in phosphorylation and/or ubiquitin-mediated degradation of cyclin D1 would provide for interesting targets for modulation of cellular proliferation through modulation of cyclin D1 levels in the cell cytoplasm. The invention is at least in part based on the discovery of these targets.

### SUMMARY OF THE INVENTION

The invention features methods and compositions for screening for agents that modulate cellular proliferation, particularly in cells that have elevated cyclin D1 (e.g., cancerous cells), where the methods provide for detection of agents that modulate phosphorylation of cyclin D1 by MAPK and/or detection of agents that modulate ubiquitination of cyclin D1 by FBXW8. The invention also features methods of controlling cellular proliferation, and agents useful in such methods.

Accordingly, in one aspect the invention provides methods for controlling cell proliferation by contacting a cell with an agent that modulates activity of a FBXW8 polypeptide, thereby controlling cell proliferation. In related aspects, the invention features methods for decreasing cell proliferation by contacting a cell with an agent, wherein the agent decreases activity of a FBXW8 polypeptide, thereby decreasing cell proliferation. In embodiments related to each of these aspects, the agent modulates activity of the FBXW8 polypeptide by modulating transcription of a nucleic acid encoding the FBXW8 polypeptide, modulating translation of a nucleic acid encoding the FBXW8 polypeptide, modulating activation of an E3 complex comprising the FBXW8 polypeptide, modulating degradation of the FBXW8 polypeptide, or modulating interaction of FBXW8 with cyclin D1 polypeptide. In further related embodiments, cell proliferation is associated with cancer or tumor growth (e.g., the cell is a cancer cell). In further related embodiments, the agent is a MAP kinase inhibitor, a Raf inhibitor, or an MEK inhibitor.

In other aspects, the invention features methods for screening a test agent for activity in modulating cell proliferation by contacting a FBXW8 polypeptide and a phosphorylated cyclin D1 polypeptide with a test agent, said contacting being under conditions suitable for interaction of a FBXW8 polypeptide and a phosphorylated cyclin D1 polypeptide to provide for ubiquitination of the phosphorylated cyclin D1 polypeptide by the FBXW8 polypeptide; and detecting the presence or absence of an effect of the test agent upon interaction between the FBXW8 polypeptide and the cyclin D1 polypeptide; where an effect of the test agent upon said interaction in the presence of the test agent as compared to the absence of the test agent indicates the test agent is capable of modulating cell proliferation.

In related embodiments, detecting of activity of a test agent in modulating interaction of FBXW8 and phosphorylated cyclin D1 is accomplished by detecting an effect of the test agent on binding of the FBXW8 polypeptide to the phosphorylated cyclin D1 polypeptide in an in vitro assay; by detecting an effect of the test agent on ubiquitination of phosphorylated cyclin D1 polypeptide by the FBXW8 polypeptide in an in vitro assay; by detecting an effect of the test agent on binding of the FBXW8 polypeptide to the phosphorylated cyclin D1 polypeptide in a cell-based assay; detecting an effect of the test agent on ubiquitination of phosphorylated cyclin D1 polypeptide by the FBXW8 polypeptide in a cell-based assay; detecting an effect of the test agent on total phosphorylated cyclin D1 polypeptide levels in a cell; detecting an effect of the test agent on total levels of cyclin D1 polypeptide in a cell; or detecting an effect of the test agent on total levels of ubiquitinated cyclin D1 in a cell. Where the assay is a ubiquitination assay, the assay may be conducted in the presence of a detectably labeled ubiquitin molecule, and said detecting the effect of the test agent on levels of detectably labeled, ubiquitinated cyclin D1 polypeptide. Where the ubiquitination assay is conducted in a cell, the cell can contain a detectably labeled ubiquitin molecule. Further, and particularly where the assay detects total cyclin D1 levels, total phosphorylated cyclin D1 levels and/or total ubiquitinated cyclin D1 levels, the effect observed in the presence of the test agent is specific for interaction of FBXW8 with phosphorylated cyclin D1 (e.g., the agent does not detectably affect MAPK activity in phosphorylation of cyclin D1, i.e., the agent is not a modulator of MAPK activity, such as an MAPK inhibitor).

In related embodiments, activity of a test agent in modulating interaction of FBXW8 and phosphorylated cyclin D1 is conducted in a cell-based assay using cells that express at least one of the FBXW8 polypeptide and the cyclin D1 polypeptide from a recombinant nucleic acid construct in the cell. In further related embodiments, at least one of the FBXW8 polypeptide and cyclin D1 polypeptide are provided as a fusion protein comprising a detectable label. The detectable label can be, for example, an immunodetectable label (e.g., a polypeptide containing a FLAG epitope), an enzymatic polypeptide (e.g., glutathione-S-transferase), or a fluorescent polypeptide (e.g., a green fluorescent polypeptide).

In other aspects, the invention features methods of screening a test agent for activity in modulating cell proliferation by contacting a MAPK polypeptide and a cyclin D1 polypeptide with a test agent, said contacting being under conditions suitable for interaction of a MAPK polypeptide and cyclin D1 polypeptide to provide for phosphorylation of the cyclin D1 polypeptide by the MAPK polypeptide; and detecting the presence or absence of an effect of the test agent upon interaction between the MAPK polypeptide and the cyclin D1 polypeptide; where an effect of the test agent upon said interaction in the presence of the test agent as compared to the absence of the test agent indicates the test agent is capable of modulating cell proliferation.

In related embodiments, detecting activity of a test agent in modulating interaction of MAPK and cyclin D1 can be accomplished by detecting an effect of the test agent on binding of the MAPK polypeptide to the cyclin D1 polypeptide in an in vitro assay; detecting an effect of the test agent on phosphorylation cyclin D1 by the MAPK polypeptide in an in vitro assay; detecting an effect of the test agent on binding of the MAPK polypeptide to the cyclin D1 polypeptide in a cell-based assay; detecting an effect of the test agent on phosphorylation of the cyclin D1 polypeptide by the MAPK polypeptide in a cell-based assay; detecting an effect of the test agent on total levels of phosphorylated cyclin D1 in a cell (where the effect is specific for interaction between MAPK and cyclin D1, e.g., the agent does not detectably affect activity of FBXW8 (e.g., the agent is not an FBXW8 inhibitor)); detecting an effect of the test agent on total levels of cyclin D1 in a cell (where the effect is specific for interaction between MAPK and cyclin D1); detecting an effect of the test agent on total levels of ubiquitinated cyclin D1 in a cell (where the effect is specific for interaction between MAPK and cyclin D, e.g., the agent does not detectably affect activity of FBXW8 (e.g., the agent is not an FBXW8 inhibitor)).

In related embodiments, activity of a test agent in modulating interaction of MAPK and cyclin D1 is conducted in a cell-based assay using cells that express at least one of the MAPK polypeptide and the cyclin D1 polypeptide from a recombinant nucleic acid construct in the cell. In further related embodiments, at least one of the MAPK polypeptide and cyclin D1 polypeptide are provided as a fusion protein comprising a detectable label. The detectable label can be, for example, an immunodetectable label (e.g., a polypeptide containing a FLAG epitope), an enzymatic polypeptide (e.g., glutathione-S-transferase), or a fluorescent polypeptide (e.g., a green fluorescent polypeptide).

In other aspects, the invention features an isolated polypeptide complex, which complexes are composed of a FBXW8 polypeptide; a Cullin polypeptide, where the Cullin polypeptide is a CUL1 polypeptide or a CUL7 polypeptide; a SKP1 polypeptide; and a phosphorylated cyclin D1 polypeptide, where the complex is capable of binding a phosphorylated cyclin D1 polypeptide. In related embodiments, at least one polypeptide of the complex is detectably labeled. In related aspects, the polypeptide complex is present in a reaction mixture.

In still other aspects, the invention features a reaction mixture having an isolated cyclin D1; and an isolated MAPK polypeptide. The reaction mixture may also contain source of phosphate for phosphorylation of cyclin D1 by MAPK, which may optionally be a source of radiolabled phosphate.

In further aspects, the invention features a method for inhibiting cell proliferation by contacting a cell with an effective amount of a small interfering nucleic acid (siNA) for at least one of an FBXW8-encoding nucleic acid, a CUL1-encoding nucleic acid, or a CUL7-encoding nucleic acid; where contacting provides for inhibition of proliferation of the cell. In related embodiments the cell is a cancerous cell. In further related embodiments, contacting is effective to inhibit growth of a tumor.

In other aspects, the invention provides a composition comprising an isolated small interfering nucleic acid (siNA), wherein the siNA comprises a sequence effective to inhibit transcription or translation of an FBXW8-encoding nucleic acid, a CUL1-encoding nucleic acid, or a CUL7-encoding nucleic acid; and a pharmaceutically acceptable carrier.

### BRIEF DESCRIPTION OF THE DRAWINGS

The patent or application file contains at least one drawing executed in color. Copies of this patent or application publication with color drawing(s) will be provided by the U.S. Patent and Trademark Office upon request and payment of necessary fee.

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1 provides a schematic overview of the phosphorylation and ubiquitination pathway involving MAPK, cyclin D1, and FBXW8.

FIG. 2 illustrates expression of cyclin D1 protein decreases during S phase in cancer cells. Panels A and B: Subcellular distribution of endogenous cyclin D1 in G1 and S phase (Magnification; ×600) in (Panel A) NIH3T3 cells (Panel B) HCT 116 colon cancer cells. Cells were rendered quiescent by serum starvation for 48 hours and were then stimulated with an addition of 10% FBS containing media to allow synchronous progression on permanox multiwell slides. Cells were fixed with paraformaldehyde at 9 hrs (NTH 3T3) or 6 hrs (HCT 116) when most of cells were in G1 phase and 21 hrs (NIH 3T3) or 15 hrs (HCT 116) when most of cells were in S phase. Subsequently cells were permeabilized by Triton X-100, and then stained with a mouse cyclin D1 monoclonal antibody followed by Alexa Fluor 594-conjugated anti-mouse IgG antibody (Red). Nuclei were visualized with Hoechst dye. Panels C and D: Expression profile of cyclin D1 protein during cell cycle progression released from quiescence in normal cells and cancer cells. Panel C: NTH 3T3 mouse, NIH3T3 human fibroblasts and CCD841 CoN normal colon epithelium. Panel D: HCT 116 and SW480 colon cancers and T98G glioblastomas. CCD841 CoN cells were synchronized at G1/G1 phase by treated with ACL-4 media without EGF for 24 hours and then stimulated by complete ACL-4 media containing EGF. Other cells were released from quiescence by serum stimulation. Samples were collected at indicated time points. Cell cycle distributions were determined by flow-cytometric cell cycle analyses and percentages of each phase were indicated with bar graphs. Western-blots were performed with cyclin D1 and β-actin antibodies respectively.

FIG. 3 illustrates that cyclin D1 is destabilized during S phase through the ubiquitin-proteasome pathway in cancers. Panels A and B: Pulse-chase analysis of cyclin D1 protein in NIH 3T3 (Panel A) and HCT 116 (Panel B) cells. When most of the populations were in G1 phase (9 hrs for NIH3T3 and 6 hrs for HCT 116) or in S phase (21 hrs for NIH3T3 and 15 hrs for HCT 116), cells were released from quiescence and then labeled with 35^{S}-methionine for 1 hour. Subsequently these cells were chased with cold methionine for the indicated times and then lysed. Cyclin D1 was immunoprecipitated and then analyzed with SDS-PAGE. Autoradiography was performed. Levels of metabolically labeled-cyclin D1 were estimated by quantitative scanning using the Quantity One (Bio-Rad) software and blotted on the graph to determine the half-life of cyclin D1. Cell cycle distribution was indicated in the tables respectively. Panel C: illustrates that the turnover of cyclin D1 protein is mediated by the ubiquitin-proteasome pathway. WI-38 and HCT 116 cells were released from quiescence by serum stimulation and treated in the presence (+) or absence (−) of MG132 for 2 hours at each time points. Western blot was performed with cyclin D1 and p-actin antibodies. Panel D: Polyubiquitination of cyclin D1 protein. HCT 116 colon cancer cells were transfected with (lane 1-3) or without (lane 4) ubiquitin cDNA and then synchronized to S phase through the sequential manipulation of serum starvation and stimulation. Cells were treated with (lane 3 and 4) or without (lane 1 and 2) 25 μM MG132 for an hour. Lysates were immunoprecipitated with either a cyclin D1 antibody (lane 2-4) or a control IgG (lane 1) and immunoblotted with a HA antibody (upper panel) or a cyclin D1 antibody (lower panel). The asterisk indicates background non-specific bands.

FIG. 4 illustrates in Panel A: Western-blot analysis using nuclear (N) and cytoplasmic (C) fraction (Fr.) protein extracted from cell lysates. Membrane was stained with Histone HI, MEK1, and cyclin D1. Panel B: Immunoprecipitation-immunoblot analysis. HCT 116 cells were transfected with ubiquitin cDNA and then synchronized to S phase through the sequential manipulation of serum starvation and stimulation. Cells were treated with Leptomycin B (LMB) for 3 hours to inhibit nuclear-to cytoplasmic localization of cyclin D1. Subsequently cells were treated with MG132 for an hour before harvesting. Nuclear protein (N) was fractionated and immunoprecipitated with a cyclin D1 antibody and immunoblotted with a HA antibody (upper panel) or a cyclin D1 antibody (lower panel). The asterisk indicates background non-specific bands.

FIG. 5 illustrates that MAPK regulates the Thr286 phosphorylation of cyclin D1 protein. Panel A: Half-life analysis of T286A and R29Q cyclin D1 (CD1) mutants and wild-type (WT) cyclin D1 protein. Stable SW480 cell lines expressing either HA-tagged cyclin D1 T286A, R29Q, or WT were generated. Cells were treated with CHX at 6 hr (G1 phase) or 15 hr (S phase) and chased for 3 hours. Immunoblot analysis was performed. Ectopically expressed T286A, R29Q, or WT CD1 was compared with the endogenous expression of cyclin D1, respectively. Asterisks indicate endogenous cyclin D1 expression. Panel B: Western blot analysis with fractionated nuclear proteins. Synchronized HCT 116 cells were released from quiescence by serum stimulation to induce re-entry into the cell cycle. Cell-cycle distribution was shown as bar graphs. Nuclear proteins were fractionated at indicated time points. Protein blot was performed with a cyclin D1, Thr286 phosphorylation-specific cyclin D1, CDK4, CDK6, GSK3β phosphorylation specific GSK3α at Tyr279 and GSK3β at Tyr216, phosphorylation specific antibodies of Rb at Ser780 and p44 and p42 ERK1/2 at Thr202/Tyr204. Panels C-E illustrate that MAPK regulates the Thr-286 phosphorylation and stability of cyclin D1 protein. Panels C and D: HA-WT CD1 (D) or Panel E: HA-CD1 T286A ectopically expressing SW480 cells were treated with highly specific small molecule inhibitors for GSK3β CDK4 and MEK. LiCl and BIO, AG12275, and U0126 were used for GSK, CDK4 and MEK/MAPK inhibitions respectively. Twenty-four hours after the treatment, cells were harvested and western-blot analyses were performed with a cyclin D1 and the p-Thr286 cyclin D1 antibodies. The phosphorylated cyclin D1 expression was magnified and normalized to its total (phosphorylated plus non-phosphorylated protein) expression and shown in the graphs in Panel C. Inhibition of the kinase activities by drugs was assessed using phosphorylation-specific antibodies for GSK3α/β, Rb and ERK1/2 on the same membrane respectively. Panel E: Cell lines expressing either HA-CD1 WT or HA-CD1 T286A were cultured in the presence (lane 1 and 4) or absence (lane 2 and 5) of serum for 24 hours. Cycling these cell lines were transfected with 7.5 μg active MEK1 and 2.5 μg pMAKS K^{k} respectively (lane 3 and 6). After 24 hours of the transfection, cells were serum-starved and cultured for a further 24 hours. Cells expressing the truncated H-2 K^{k} were used for protein blot (lane 3 and 6). Levels of total and phosphorylated cyclin D1 were determined. Panel F: Inhibition of GSK3 activities did not have any effect on expression of cyclin D1. HA-WT CD1 SW480 cells were treated with combining U0126 with LiCl. Arrowheads in Panels C, E, and F; double arrowheads in Panels Dand E or asterisks in Panels A and C-F indicate HA-CD1 WT, HA-CD1 T286A, or endogenous CD1 expression respectively.

FIG. 6 illustrates that MAPK phosphorylates cyclin D1 at Thr286, which triggers subsequent ubiquitination. Panel A: Identification of the D-domain within CD1 protein. The illustration of a full-length human CD1 shows the region of the D-domain and the MAPK phosphorylation site Thr286 (T286) with a solid bar and an arrow respectively. The amino acid sequence of the D-domain within CD1 is indicated in alignment with other known MAPK-docking sites of various ERK substrates. The doublet of basic (+) and nonpolar (φ) amino acids are conserved residues in the core D-domain motif L/I/V-X-L/I/V. Amino acid positions of the most 5′ residues of the D-domains are indicated with numbers in the left of each amino acid sequence respectively. Panels B and C: p42 ERK2 in vitro kinase assays for cyclin D1 (upper panels). Immuno-blotting (IB) analyses stained with a cyclin D1 antibody (b), or a GST antibody (c) were provided as a reference to show the amounts of substrates. Panel B: Wild type or T286A mutant recombinant GST-full length cyclin D1 protein was mixed with ³²P-ATP in the kinase assay reaction buffer in the presence or absence of purified EPvK2. Reactions were performed at 30° C. for 30 min and stopped by adding sample loading buffer. Samples were separated with SDS-PAGE and then ³²P-uptake was detected by autoradiography. Panel C: GST alone (lane 1), GST-C-terminal WT cyclin D1 fusion protein that retains the biding site of MAPK (amino acids 165-295, lane 2), T286A (lane 3) and a complete deletion of the D-domain (ΔD, lane 4) were used. Panel D: immunoprecipitation and immunoblotting analysis following ectopic expression of Flag-tagged ERK2 together with either HA-tagged WT or ΔD CD1 in HCT 116 colon cancer cells. Western blot for input controls (lysate) were provided.

FIG. 7 illustrates western blot analysis following transfection of various forms of HA-tagged cyclin D1 expression vectors in HCT 116 colon cancer cells.

FIG. 8 illustrates that MAPK regulates stability and relocalization of cyclin D1 protein. Panels A and B: Half-life analysis of HA-CD1 WT after exposure to U0126. Panel A: Exponentially growing HA-WT cyclin D1 SW480 cells were exposed to U0126 for 24 hours and subsequently treated with CHX and chased for 3 hours. Cells were harvested at different times and protein blot was performed (upper panel). Cyclin D1 expression was quantified and the half-life was calculated respectively. Panel B: Western blot analysis. Panels C and D: NIH 3T3 cells stably expressing the ΔB-Raf:ER^{TAM} were treated with 4-hydroxy-tamoxifen (4-HT). Panel C: Western blot analysis after 4-HT treatment. Panel D: Expression profiles of cyclin D1 protein and MAPK (ERK1/2) during cell cycle progression from quiescence. Cells were serum starved for 48 hours and then stimulated by the addition of 10% FBS containing media with (+) or without (−) 10 nM 4-HT. Panel E: Half-life analysis of endogenous cyclin D1 protein after MAPK induction. Exponentially growing cells were cultured in the presence (+) or absence (−) of 10 nM 4-HT and subsequently treated with CHX and chased for 3 hours. A half-life of cyclin D1 protein was calculated.

FIG. 9 illustrates that MAPK directly binds to cyclin D1 through the MAPK docking site (D-domain) within cyclin D1 and phosphorylates it specifically at Thr286. Panel A: p42 ERK2-associated GST-cyclin D1 (CD1) in vitro kinase assays (upper panels). Purified ERK2 phosphorylates cyclin D1 in vitro. Immuno-blotting (IB) analyses stained with a GST antibody (lower panels) were provided as a reference to show loading conditions of various forms of GST-CD1 fusion proteins. (A) GST alone (lane 1), GST-human (h) CD1 amino acids (A.A.) from 165 to 295 (165-295, lane 2), GST-human (h) or mouse (m) CD1 A.A. from 255 to 295 (255-295, lane 3 and 4) were used. Panel B: Western blot analysis following transfection of various forms of HA-tagged cyclin D1 expression vectors in both NIH 3T3 mouse fibroblast (left) and HCT 116 colon cancer (right) cells. In lane 5 and 10, Cells were treated with 5 μM of a highly specific GSK3β inhibitor BIO for further 24 hours following transfcetion of the ΔD CD1 mutant.

FIG. 10 illustrates in vitro ubiquitination assays using HeLa cell extracts Fraction II as a source of the enzymes necessary to conjugate ubiquitin to substrates and ATP. Panel A: Results in which GST-full length CD1 WT, T286A, or ΔD were used for a reaction either with (lane 3-5) or without (lane 1-2) recombinant ERK2. Samples were separated by SDS-PAGE and immunoblotted with a cyclin D1 antibody. ATP was added to all lanes but not Ubiquitin to lane 1. Panel B: Results in which GST-full length CD1 WT was used with or without ubiquitin. After separated with SDS-PAGE, immunobloting were performed with a cyclin D1 (lane 1, 2) or a ubiquitin antibody (lane 3, 4).

FIG. 11 illustrates that ERK/MAPK is identified as the major kinase that is responsible for the stability of cyclin D1 protein. Panels A-F: Pulse-chase analysis of cyclin D1 protein in HCT 116 cells after exposure to the MEK inhibitor U0126 (A-C) or the GSK3 inhibitor BIO (D-F). Exponentially growing HCT 116 cells were treated with either DMSO or 10 μM of U0126 (A-C) for 30 minutes, or DMSO or 5 μM of BIO (D-F) for 24 hours. Panels A and D: Cells were pulse-labeled with ³⁵S-methionine for 1 hour. Subsequently these cells were chased with cold methionine for the indicated times, and then lysed respectively. Cyclin D1 was immunoprecipitated and then analyzed with SDS-PAGE. Autoradiography was performed. Levels of metabolically labeled-cyclin D1 were estimated by quantitative scanning using the Quantity One (Bio-Rad) software and blotted on the graph to determine the half-life of cyclin D1. Panels B and E: Western blot analysis. The membrane was blotted with a cyclin D1 Thr286 phosphorylation specific (pThr286), total cyclin D1, a phosphorylation specific ERK, and total ERK antibodies (B), or a pThr286, cyclin D1, a GSKα/β phosphorylation specific, and GSK3β antibodies (E). Panels C and F: Cell cycle distributions were shown. Panel G: Western blot analysis following transfection of various forms of HA-tagged cyclin D1 expression vectors in both NIH 3T3 mouse fibroblast (left) and HCT 116 colon cancer (right) cells. In lane 5 and 10, cells were treated with 5 μM of BIO for further 24 hours following transfection of the ΔD CD1 mutant.

FIG. 12 illustrates the relocalization of cyclin D1 into the cytoplasm as cells proceed into S phase facilitates phosphorylation of cyclin D1 through ERK/MAPK. Panels A-C: Immunofluorescence analysis. Nuclei were visualized with Hoechst dye. Cell cycle distributions were determined by flow-cytometric cell cycle analyses. Panel A: Subcellular localization of cyclin D1. HCT 116 colon cancer cells were treated with 5 μM of the GSK3 inhibitor BIO for 24 hours. Cells were pulse-labeled with bromodeoxyuridine (BrdU) for an hour in the presence or absence of BIO. Subsequently cells were fixed with ethanol and then stained with a rabbit cyclin D1 polyclonal antibody followed by Alexa Fluor 594-conjugated anti-rabbit IgG antibody (Red) and a mouse BrdU-FITC antibody (Upstate). Panel B: Subcellular localization of the E3 ligase. HCT 116 colon cancer cells were transfected with V5 epitope-tagged FBXW8 pcDNA3. Twenty-four hours later cells were synchronized by sequential manipulation of serum starvation and stimulation. At 6 hrs (G1 phase) or 15 hrs (S phase) after serum stimulation, cells were fixed and immunofluorescence was performed with a V5 epitope tag antibody followed by Alexa Fluor 488-conjugated anti-mouse IgG antibody (Green) and a rabbit cyclin D1 polyclonal antibody followed by Alexa Fluor 594-conjugated anti-rabbit IgG antibody (Red). Panel C: Subcellular localization of Thr286 phosphorylated cyclin D1 (pThr286 cyclin D1) and phosphorylated form of ERK (pERK). HCT 116 cells were synchronized by sequential manipulation of serum starvation and stimulation. At 6 hrs (G1 phase) or 15 hrs (S phase), cells were fixed and immunofluorescence was performed with a cyclin D1 Thr286 phosphorylation specific antibody followed by Alexa Fluor 594-conjugated anti-rabbit IgG antibody (Red) and an ERK phosphorylation specific antibody followed by Alexa Fluor 488-conjugated anti-mouse IgG antibody (Green). Panel D: Immunoprecipitated-immunoblotting analysis. Panel E: Cell cycle distributions. Panels D and E: Exponentially growing HCT 116 colon cancer cells were transiently transfected with Flag epitope-tagged FBXW8 DNA plasmid together with HA-tagged cyclin D1 and CDK4 expression vectors. Twenty-four hours later cells were treated with DMSO (−) or 10 μM of U0126 (+) for 30 minutes and then harvested. Cell lysates were immunoprecipitated with either a Flag (FBXW8) antibody or a control IgG and immunoblotted with cyclin D1, pThr286, and Flag (FBXW8) antibodies (left panel). Western blot for input controls (lysate) were provided (right panel).

FIG. 13 illustrates FBXW8 ubiquitinates cyclin D1 in a Thr286 phosphorylation dependent manner. Panel A: Immunoprecipitation (IP)-immunoblotting (IB) analysis (left). Protein from exponentially growing HCT 116 colon cancer cells was precipitated with antibodies to cyclin D1 or IgG. Immunoprecipitates were subjected to SDS-PAGE and sequentially blotted with cyclin D1, CDK4, SKP1, CUL1 and CUL7 antibodies. IB analysis with 5% of total cell lysates was provided (right). Panel B: Immunoblot analysis following depletion of SKP1 expression for 48 hours through small interfering (si) RNA double-strand oligonucleotides in HCT 116 cells. Non-targeting siRNA (Control) and mock transfection (−) served as controls. Panel C:IP-IB analysis. Twenty-six retrieved full-length encoding cDNAs were cloned into V5 or Flag epitope tag expression vectors, respectively. These V5 or Flag-tagged F-box protein DNA plasmids were transfected together with HA-tagged cyclin D1 (HA-Cyc D1) and CDK4 expression vectors into T98G glioblastoma cells, respectively. Cells were collected 24 hours later. The samples were precipitated with a HA epitope tag antibody. Immunoprecipitates were subjected to SDS-PAGE and subsequently stained with V5 or Flag (F-box proteins), HA (Cyc D1) antibodies. IB analysis with 10% of total cell lysates was provided (bottom).

FIG. 14 illustrates: Panel A: Immunoprecipitation-immunoblotting analysis (top). V5-tagged F-box protein DNA plasmids were transiently transfected together with either HA-tagged cyclin D1 (Cyc D1) wild type (WT) or T286A mutant, and CDK4 expression vectors in T98G glioblastoma cells respectively. Samples were precipitated with a HA epitope-tag antibody. Immunoprecipitates were subjected to SDS-PAGE and subsequently blotted with V5 (F-box proteins) and HA (Cyc D1) antibodies. IB analysis with 10% of total cell lysates was provided (bottom). Panel B: In vitro binding assay. ³⁵S-labeled in vitro translated FBXW8, FBXL12 or β-TRCP was incubated with rabbit reticulocyte cell extracts and beads coupled to either the Thr286 phosphorylated cyclin D1 peptide (Cyclin D1-P; lane 2) or unphosphorylated cyclin D1 peptide (lane 1, Cyclin D1) overnight at 4° C. Beads were extensively washed with 0.5% NP-40 Tris-C1 buffer. Associated proteins were eluted with the sample bufferand separated by SDS-PAGE. The lane 3 or 6 contains 50% input of each ³⁵S-labeled in vitro-translated product. Panel C: In vitro ubiquitination assay. In vitro-translated F-box proteins with recombinant GST-full-length cyclin D1 (GDI) wild type, HeLa cell extracts Fraction II with ATP, Ubiquitin and ERK2, and In vitro-translated either SKP1, RBX1 and CUL1, or SKP1, RBX1 and CUL7 proteins and were incubated at 30° C. for 2 hours. Samples were separated by SDS-PAGE and immunoblotted with a cyclin D1 antibody.

FIG. 15 illustrates an in vitro ubiquitination assay. In vitro translated F-box proteins with recombinant GST-β-catenin (Upstate), HeLa cell extracts Fraction II with ATP, Ubiquitin, GSK3 β and in vitro-translated SKP1, RBX1 and CUL1 were incubated at 30° C. for 2 hours. Samples were separated by SDS-PAGE and immunoblotted with a β-catenin antibody.

FIG. 16 illustrates: Panel A: In vitro polyubiquitination of cyclin D1 through the SCF-like (SCFL) complex FBXW8 (SKP1-CUL7-FBXW8-RBX1/SCFL^{FBXW8}). WT or T286A GST-CD1 was incubated in the presence (lanes 1, and 3) or absence (lane 2) of purified ERK2 at 30° C. for 2 hours. Samples were separated by SDS-PAGE and immunoblotted with a cyclin D1 antibody. Asterisks indicate non-specific bands. Panel B: Reconstitution of polyubiquitination of cyclin D1 through SCFL^{FBXW8} in vitro using purified E1 and E2. GST-WT CD1 was incubated with recombinant SCFL^{FBXW8} in the presence or absence of E1 and E2/UbcH5C. Samples were separated by SDS-PAGE and immunoblotted with a cyclin D1 antibody.

FIG. 17 illustrates that the stability of cyclin D1 protein is regulated by the complexes of FBXW8 through the ubiquitin-proteasome pathway. Panels A and B: Immunoblot analysis. HCT 116 cells were infected with a retrovirus expressing the FBXW8 (A), the ΔF mutant form (ΔF FBXW8, Panel B) or a control retrovirus expressing GFP (A, B). Forty-eight hours later, cells were harvested and a western blot analysis was performed with antibodies to cyclin D1, cyclin E, Flag (FBXW8 and ΔF FBXW8), GFP and β-actin. Panel C: Immunoprecipitation (IP)-immunoblotting (IB) analysis. Empty (mock) or Flag-tagged WT FBXW8, and ΔF FBXW8 DNA plasmids were transiently transfected in T98G glioblastoma cells. Samples were precipitated with a Flag epitope tag antibody. Immunoprecipitates were subjected to SDS-PAGE and subsequently blotted with antibodies to cyclin D1, SKP1, CUL1, CUL7, RBX1 and Flag (F-box proteins). Panel D: Immunoblot analysis following depletion of FBXW8 expression for 48 hours through siRNA in HCT 116 colon cancer cells. Non-targeting siRNA (Control) and mock transfection (−) were served as controls.

FIG. 18 illustrates that knockdown of FBXW8, or its partner CUL1 or CUL7 expression through siRNA stabilizes cyclin D1 expression in HCT 116 colon cancer cells. Panels A and B: Immunoblot analysis (A) and RT-PCR analysis (B) following depletion of CUL1, CUL7 or FBXW8 expression for 48 hours through siRNA or mismatch (MM) oligonucleotides in HCT 116 colon cancer cells. Non-targeting siRNA (Control) and mock transfection (−) were served as controls. Relative gene expression is shown (B). Panels C and D: Pulse-chase analysis of cyclin D1 following depletion of CUL1, CUL7 or FBXW8 expression for 48 hours through siRNA in HCT 116 cells. Control cells were treated with non-targeting siRNA. Cells were pulse-labeled with ³⁵S-methionine for an hour, chased with cold methionine for the indicated times, and then lysed. Cyclin D1 was immunoprecipitated and then analyzed with SDS-PAGE. Levels of metabolically labeled-cyclin D1 were estimated by quantitative scanning using the Quantity One software (Bio-Rad) and blotted on the graph to determine the half-life of cyclin D1 (D).

FIG. 19 illustrates: Western blot analysis (Panel A). HCT 116 cells were infected with a retrovirus expressing a control empty vector (mock), or a dominant-negative (DN) FBXW8 or SKP2 ΔF FBXW8 or ΔF SKP2) for 48 hours. Colony-formation assay on HCT 116 cells infected with a control empty vector (mock), DN FBXW8 or DN SKP2, respectively (Panel B). These vectors express the neomycin gene. Fourteen days after infection and growth in G418, cells were stained with 0.5% crystal violet containing 20% ethanol.

FIG. 20 illustrates that Cyclin D1 degradation in the cytoplasm is essential for cell proliferation. Panel A: Viable cell number from HCT 116 colon cancer cells after knocking down FBXW8, CUL1, or CUL7 through siRNA double-stranded oligonucleotides. siRNAs were transfected on days 0, 1, 2, and 4. Cells were collected on the indicated days (0, 1, 2, 3, 4, 5) and stained with trypan blue. Cell numbers were counted with a haemocytometer. Panel B: Western blot analysis with total cyclin D1, cyclin D1 pThr286, CDK4, Histone HI, MEK1 and Rb antibodies. HCT 116 cells were treated with either control (Cont) or FBXW8 (W8) siRNA for 72 hours. Subsequently, samples were fractionated into nuclear or cytoplasmic proteins. CDK4-associated GST-Rb in vitro kinase assay using nuclear protein (bottom). Panel C: Generation of cyclin D1 ecdysone-inducible (IND) system in HCT 116 cells. Ectopic expression of HA-tagged T286A was induced in by 10 μM Ponasterone A (Pon A). Panel D: Colony formation assay. One hundred single cells from T286A IND HCT116 were cultured in the presence (+) or absence (−) of Pon A, and control (Cont) or FBXW8 siRNA. Cells were cultured for 2 weeks, and stained with 0.5% crystal violet containing 20% ethanol.

FIG. 21 is a schematic of a model of ubiquitination of cyclin D1 through the complex containing FBXW8.

FIG. 22 illustrates the generation of a Thr286 phosphorylation-specific polyclonal antibody for cyclin D1 protein. Proteins from HCT 116 colon cancer cells were precipitated with a cyclin D1 (CD1) antibody and subsequently incubated in the presence or absence of X phosphatase (lanes 1 and 2). Either HA-tagged WT or T286A CD1 expression vector was transfected in HCT 116 cells respectively (lane 3 and 4). Westen-blot was performed with a Thr286 phosphorylation-speciflc antibody (p-Thr286) or a cyclin D1 antibody.

### DEFINITIONS

By “FBXW8” or “FBXW8 polypeptide” is meant an F-box and WD-40 domain protein 8 (also known as F-box/WD-repeat protein 8, F-box only protein 29, FBW6, FBW8, FBX29, FBXO29, FBXW6, MGC33534). In embodiments of particular interest, the FBXW8 polypeptide is a mammalian FBXW8 polypeptide, with human FBXW8 polypeptide being of particular interest.

By “CUL1” or “Cullin 1 polypeptide” is meant a polypeptide that associates in a complex with an FBXW8 polypeptide and an SKP1 polypeptide to form an E3 ubitquitin ligase which mediates ubiquitination of phosphorylated cyclin D1. In embodiments of particular interest, the CUL1 polypeptide is a mammalian CUL1 polypeptide, with human CUL1 polypeptide being of particular interest.

By “CUL7” or “Cullin 7 polypeptide” is meant a polypeptide that associates in a complex with an FBXW8 polypeptide and an SKP1 polypeptide to form an E3 ubitquitin ligase which mediates ubiquitination of phosphorylated cyclin D1. In embodiments of particular interest, the CUL7 polypeptide is a mammalian CUL7 polypeptide, with human CUL7 polypeptide being of particular interest.

By “SKP1” or “S-phase Kinase-associated Protein I” polypeptide is meant a polypeptide that associates in a complex with an FBXW8 polypeptide and either a CUL1 or CUL7 polypeptide to form an E3 ubitquitin ligase which mediates ubiquitination of phosphorylated cyclin D1. In embodiments of particular interest, the SKP1 polypeptide is a mammalian SKP1 polypeptide, with human SKP1 polypeptide being of particular interest.

By “MAPK” or “MAPK polypeptide” is meant a Mitogen-Activated Protein Kinase protein which specifically phosphorylates cyclin D1 at threonine 268 (Thr268). MAPK polypeptide referred to herein is also known in the literature as p44 ERK1; p44ERK2; ERK; p38; p40; p41; ERK2; ERT1; MAPK2; PRKM1; PRKM2; P42MAPK; and p41mapk. In embodiments of particular interest, the MAPK polypeptide is a mammalian MAPK polypeptide, with human MAPK polypeptide being of particular interest.

By “cyclin D1 polypeptide” (also known as BCL1, BCL-1 oncogene, cyclin D1, D11S287E, G1/S-specific cyclin D1, HGNC:988, PRAD1, PRAD1 oncogene, U21B31) which is a substrate for phosphorylation by MAPK and for ubiquitination by an FBXW8-containing E3 ligase (FBXW8-CUL1-SKP1 or FBXW80CUL7-SKP1). Where the cyclin D1 polypeptide serves as a substrate for MAPK phosphorylation at threonine residue 286 (Thr286), e.g., a polypeptide comprising at least amino acid residues 255 to 295 from the C-terminus of the cyclin D1 polypeptide. In embodiments of particular interest, the cyclin D1 polypeptide is a mammalian cyclin D1 polypeptide, with human cyclin D1 polypeptide being of particular interest.

“Phosphorylated cyclin D1 polypeptide” as used herein, particularly in claims directed to methods of screening for modulators of cyclin D1 ubiquitination, refers to a phosphorylated cyclin D1 which is a suitable substrate for FBXW8-mediated ubiquitination of cyclin D1 (e.g., a cyclin D1 polypeptide phosphorylated at threonine 286).

The term “interaction”, as used in the context of interaction between a MAPK and cyclin D1, or interaction between phosphorylated cyclin D1 and an FBXW8-containing E3 ligase, refers to binding or other association between the polypeptides which facilitates an enzymatic reaction to occur between an enzyme and its substrate (e.g., phosphorylation of cyclin D1 by MAPK, or ubiquitination of cyclin D1 by FBXW8) under suitable conditions. Interaction can be detected directly (e.g., by detecting binding of FBXW8 and phosphorylated cyclin D1, or binding of cyclin D1 and MAPK) or indirectly by assaying a product of a reaction that occurs as a result of the interaction (e.g., ubiquitinated cyclin D1 as a result of FBXW8 and phosphorylated cyclin D1; phosphorylated cyclin D1 as a result of interaction of MAPK and cyclin D1).

By “having a defect in a polypeptide” or “a defective polypeptide”, as in the context of a cell having a defective FBXW8 or defective MAPK, is meant that the cell exhibits a phenotype associated with decreased or no detectable activity of the polypeptide. For example, a cell having a defect in a FBXW8 polypeptide has decreased or no detectable activity in ubiquitination of phosphorylated cyclin D1. In another example, a cell having a defect in a MAPK polypeptide has decreased or no detectable activity in phosphorylation of cyclin D1. The defect in the polypeptide may be due to, for example, decreased expression of a nucleic acid encoding the polypeptide, expression of a modified polypeptide (e.g., as in a polypeptide fragment lacking all or a portion of a functional domain required for activity, a polypeptide mutant having reduced or no detectable activity (including dominant negative mutants), and the like). The dominant negative mutant of FBXW8 as described herein is an example of a defective polypeptide.

By “test agent” or “candidate agent”, “candidate”, “candidate modulator”, “candidate ubiquitination modulator”, “candidate phosphorylation modulator” or grammatical equivalents herein, which terms are used interchangeably herein, is meant any molecule (e.g. proteins (which herein includes proteins, polypeptides, and peptides), small (i.e., 5-1000 Da, 100-750 Da, 200-500 Da, or less than 500 Da in size), or organic or inorganic molecules, polysaccharides, polynucleotides, etc.) which are to be tested for activity in modulating an activity associated with cellular proliferation and mediated through cyclin D1 (e.g., phosphorylation cyclin D1, or ubiquitination of cyclin D1). Further exemplary test agents are described herein.

By “screen” or “screening” (as used in the context of the methods to identify a test agent having a desired activity) is meant that a test agent is subjected to an assay to determine the presence of absence of an activity of interest (e.g., modulation of interaction between FBXW8 and phosphorylated cyclin D1; modulation of interaction between MAPK and cyclin D1, and the like).

By “modulate” is meant that a cellular phenotype (e.g., cell proliferation) and/or activity of a gene product increased (e.g., up-regulated) or decreased (e.g., down-regulated) in the presence of a modulator (e.g., test agent, e.g., siNA), such that cellular phenotype, gene expression, mRNA or protein level, or gene product activity is greater than or less than that observed in the absence of the modulator. The context of use of the term will make it apparent as to whether increase or decrease in the relevant phenomenon is desired. For example, in the context of inhibiting cellular proliferation (e.g., as in inhibition of growth of cancerous cells) through modulating MAPK phosphorylation of cyclin D1 and/or FBXW8-mediated ubiquitination of cyclin D1, a desired “modulator” is one that inhibits cellular proliferation by inhibiting MAPK phosphorylation and/or inhibiting FBXW8-mediated cyclin D1 degradation (e.g., by inhibiting cyclin D1 ubiquitination).

By “inhibit”, “down-regulate”, or “reduce”, it is meant that the cellular phenotype, gene expression, or mRNA level, protein level, or activity of one or more proteins or protein subunits, in the presence of a test agent is reduced below that observed in the absence of the test agent. In general, an inhibitory agent generally reduces an activity of interest (e.g., cellular proliferation, an enzymatic activity (e.g., cyclin D1 phosphorylation or ubiquitination), expression of a target gene) by at least 20%, e.g., at least 30%, at least 40%, at least 50%, at least 60%, at least 70%, at least 80%, at least 90%, at least 95%, up to about 99% or 100% in an assay, as compared to the same assay performed in the absence of the compound. In some embodiments, e.g., where inhibition of cellular proliferation using an siNA is involved, inhibition, down-regulation or reduction with an siNA molecule is below that level observed in the absence of the siNA molecule or in the presence of a negative control (e.g., an inactive or attenuated molecule, or an siNA molecule with scrambled sequence and/or mismatches).

By “nucleic acid” herein is meant either DNA or RNA, or molecules which contain both deoxy- and ribonucleotides. The nucleic acids include genomic DNA, cDNA and oligonucleotides including sense and anti-sense nucleic acids. Also siNAs, such as siRNAs, are included. Such nucleic acids may also contain modifications in the ribose-phosphate backbone to increase stability and half life of such molecules in physiological environments.

The nucleic acid may be double stranded, single stranded, or contain portions of both double stranded or single stranded sequence. As will be appreciated by those in the art, the depiction of a single strand (“Watson”) also defines the sequence of the other strand (“Crick”). By the term “recombinant nucleic acid” herein is meant nucleic acid, originally formed in vitro, in general, by the manipulation of nucleic acid by endonucleases, in a form not normally found in nature. Thus an isolated nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell or organism, it will replicate non-recombinantly, i.e. using the in vivo cellular machinery of the host cell rather than in vitro manipulations; however, such nucleic acids, once produced recombinantly, although subsequently replicated non-recombinantly, are still considered recombinant for the purposes of the invention.

Nucleic acid sequence identity (as well as amino acid sequence identity) is calculated based on a reference sequence, which may be a subset of a larger sequence, such as a conserved motif, coding region, flanking region, etc. A reference sequence will usually be at least about 18 residues long, more usually at least about 30 residues long, and may extend to the complete sequence that is being compared. Algorithms for sequence analysis are known in the art, such as BLAST, described in Altschul et al. (1990), J. Mol. Biol. 215:403-10 (using default settings, i.e. parameters w=4 and T=17).

Where a nucleic acid is said to hybridize to a recited nucleic acid sequence, hybridization is under stringent conditions. An example of stringent hybridization conditions is hybridization at 50° C. or higher and 0.1×SSC (15 mM sodium chloride/1.5 mM sodium citrate). Another example of stringent hybridization conditions is overnight incubation at 42° C. in a solution: 50% formamide, 5×SSC (150 mM NaCl, 15 mM trisodium citrate), 50 mM sodium phosphate (pH7.6), 5× Denhardt's solution, 10% dextran sulfate, and 20 μg/ml denatured, sheared salmon sperm DNA, followed by washing the filters in 0.1×SSC at about 65° C. Stringent hybridization conditions are hybridization conditions that are at least as stringent as the above representative conditions, where conditions are considered to be at least as stringent if they are at least about 80% as stringent, typically at least about 90% as stringent as the above specific stringent conditions. Other stringent hybridization conditions are known in the art and may also be employed to identify nucleic acids of this particular embodiment of the invention.

Similarly, “polypeptide” and “protein” as used interchangeably herein, and can encompass peptides and oligopeptides. Where “polypeptide” is recited herein to refer to an amino acid sequence of a naturally-occurring protein molecule, “polypeptide” and like terms are not necessarily limited to the amino acid sequence to the complete, native amino acid sequence associated with the recited protein molecule, but instead can encompass biologically active variants or fragments, including polypeptides having substantial sequence similarity or sequence identify relative to the amino acid sequences provided herein. In general, fragments or variants retain a biological activity of the parent polypeptide from which their sequence is derived (e.g., activity in phosphorylating cyclin D1 where the parent polypeptide is MAPK; activity in ubiquitination of cyclin D1 where the parent polypeptide is FBXW8). It should be noted that, as will be clear from the context, reference to cyclin D1, FBXW8, MAPK, CUL1, CUL7 and SKP1 is intended to refer to cyclin D1 polypeptide, FBXW8 polypeptide, MAPK polypeptide, CUL1 polypeptide, CUL7 polypeptide, and SKP1 polypeptide.

As used herein, “polypeptide” refers to an amino acid sequence of a recombinant or non-recombinant polypeptide having an amino acid sequence of i) a native polypeptide, ii) a biologically active fragment of an polypeptide, or iii) a biologically active variant of an polypeptide. Polypeptides useful in the invention can be obtained from any species, e.g., mammalian or non-mammalian (e.g., reptiles, amphibians, avian (e.g., chicken)), particularly mammalian, including human, rodenti (e.g., murine or rat), bovine, ovine, porcine, murine, or equine, preferably rat or human, from any source whether natural, synthetic, semi-synthetic or recombinant. In general, polypeptides comprising a sequence of a human polypeptide are of particular interest. For example, “Human FBXW8 polypeptide” refers to the amino acid sequences of isolated human FBXW8 polypeptide obtained from a human, and is meant to include all naturally-occurring allelic variants, and is not meant to limit the amino acid sequence to the complete, native amino acid sequence associated with the recited protein molecule.

A “variant” of a polypeptide is defined as an amino acid sequence that is altered by one or more amino acids (e.g., by deletion, addition, insertion and/or substitution). Generally, “addition” refers to nucleotide or amino acid residues added to an end of the molecule, while “insertion” refers to nucleotide or amino acid residues between residues of a naturally-occurring molecule. The variant can have “conservative” changes, wherein a substituted amino acid has similar structural or chemical properties, e.g., replacement of leucine with isoleucine. More rarely, a variant can have “nonconservative” changes, e.g., replacement of a glycine with a tryptophan. Similar minor variations can also include amino acid deletions or insertions, or both. Guidance in determining which and how many amino acid residues may be substituted, added, inserted or deleted without abolishing biological or immunological activity can be found using computer programs well known in the art, for example, DNAStar software.

The term “isolated” indicates that the recited material (e.g, polypeptide, nucleic acid, etc.) is substantially separated from, or enriched relative to, other materials with which it occurs in nature (e.g., in a cell). A material (e.g., polypeptide, nucleic acid, etc.) that is isolated constitutes at least about 0.1%, at least about 0.5%, at least about 1% or at least about 5% by weight of the total material of the same type (e.g., total protein, total nucleic acid) in a given sample.

“Treating” or “treatment” of a condition or disease includes: (1) preventing at least one symptom of the conditions, i.e., causing a clinical symptom to not significantly develop in a mammal that may be exposed to or predisposed to the disease but does not yet experience or display symptoms of the disease, (2) inhibiting the disease, e.g., arresting or reducing the development of the disease or its symptoms, or (3) relieving the disease, i.e., causing regression of the disease or its clinical symptoms.

A “therapeutically effective amount” or “efficacious amount” means the amount of a compound that, when administered to a mammal or other subject for treating a disease, is sufficient to effect such treatment for the disease. The “therapeutically effective amount” will vary depending on the compound, the disease and its severity and the age, weight, etc., of the subject to be treated.

The terms “subject” and “patient” mean a member or members of any mammalian or non-mammalian species that may have a need for the pharmaceutical methods, compositions and treatments described herein. Subjects and patients thus include, without limitation, primate (including humans), canine, feline, ungulate (e.g., equine, bovine, swine (e.g., pig)), avian, and other subjects. Humans and non-human animals having commercial importance (e.g., livestock and domesticated animals) are of particular interest.

“Mammal” means a member or members of any mammalian species, and includes, by way of example, canines; felines; equines; bovines; ovines; rodentia, etc. and primates, particularly humans. Non-human animal models, particularly mammals, e.g. primate, murine, lagomorpha, etc. may be used for experimental investigations.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound (e.g., phenylglycine-containing compound or sulfonamide containing compound) employed and the effect to be achieved, and the pharmacodynamics associated with each compound in the host.

A “pharmaceutically acceptable excipient,” “pharmaceutically acceptable diluent,” “pharmaceutically acceptable carrier,” and “pharmaceutically acceptable adjuvant” means an excipient, diluent, carrier, and adjuvant that are useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. “A pharmaceutically acceptable excipient, dileuent, carrier and adjuvant” as used in the specification and claims includes both one and more than one such excipient, dileuent, carrier, and adjuvant.

As used herein, a “pharmaceutical composition” is meant to encompass a composition suitable for administration to a subject, such as a mammal, especially a human. In general a “pharmaceutical composition” is sterile, and preferably free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intracheal and the like.

“Ubiquitinated” or “ubiquitination” in reference to a protein is meant to encompass modification of a polypeptide by conjugation to a ubiquitin (Ub) or a ubiquitin-like modifier (UbI).

By “ubiquitin agents” is meant a molecule involved in ubiquitination, most frequently enzymes. Ubiquitin agents can include ubiquitin activating agents, ubiquitin ligating agents and ubiquitin conjugating agents. In addition, ubiquitin agents can include ubiquitin moieties as described below. In addition, de-ubiquitylation agents (e.g. proteases that degrade or cleave ubiquitin or polyubiquitin chains) find use in the invention.

Other definitions of terms appear throughout the specification.

### DETAILED DESCRIPTION OF THE INVENTION

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the polypeptide” includes reference to one or more polypeptides and equivalents thereof known to those skilled in the art, and so forth.

It is further noted that the claims may be drafted to exclude any element which may be optional. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Overview

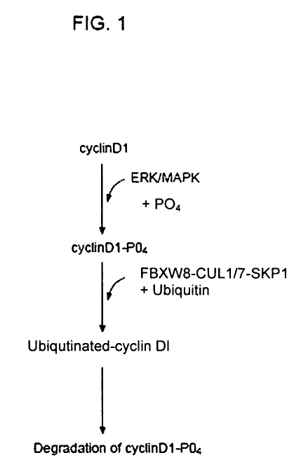

Cyclin D1 degradation is required for cell proliferation, including proliferation of cancer cells. The inventors have demonstrated that the MAPK signaling cascade promotes cyclin D1 phosphorylation at Thr-286 and that MAPK is the major kinase which specifically phosphorylates cyclin D1 at Thr-286. Phosphorylated cyclin D1 is polyubiquitinated and degraded through the 26S proteasome pathway. (for a schematic, see FIG. 1)

Furthermore, the inventors have demonstrated that the E3 ubiquitin ligase which specifically interacts with cyclin D1 contains FBXW8 F-box protein. The FBXW8 F-box protein associates with either CUL1 or CUL7 (referred to herein as “CUL1/CUL7”) and SKP1 to form an SCF-like complex which recognizes cyclin D1 in a phosphorylation-dependent manner. The ubiquitination of cyclin D1 is regulated by the FBXW8-CUL1/CUL7-SKP1 complex. The inventors have further demonstrated that inhibiting activity of FBXW8 F-box protein or either of CUL1 or CUL7 through RNA interference or a dominant-negative mutant causes accumulation of stabilized cyclin D1 in the cytoplasm, which results in the reduction of cancer cell proliferation.

FBXW8 Protein (F-BOX WD-40 Domain Protein 8)

FBXW8 (F-Box, WD-40 domain protein; also known as (FBW6, FBW8, FBX29, FBXO29, MGC33534) contains a WD-40 domain and an F-box motif. The consensus sequence of an F-box motif is described in Bai et al., 1996, Cell 86:263, incorporated herein by reference in its entirety. FBXW8 protein interacts with SKP1 and either CUL1 or CUL7 to form an ubiquitin E3 ligase complex to ubiquitinate phosphorylated cyclin D1, which then leads to degradation.

The FBXW8 protein may be produced by any method known in the art Exemplary methods are specifically described below. In one embodiment, the subject FBXW8 protein is made by performing a reverse transcriptase-polymerase chain reaction (RT-PCR) using total RNA from cells, for example, HEK 293, HCT 116 or WI-38 cells to obtain the FBXW8 F-box protein gene. The retrieved full-length cDNA is then cloned into pFB retrovirus expression vector (Stratagene) and transfected to amphotropic phoenix cells. The supernatant was harvested for 48-72 hrs after transfection, filtered, and stored at −80° C. Cells were infected with a virus media containing 8 μg/ml polybrene for 4 hours then subsequently replaced with fresh media and cultured for further 48 hours.

DNA sequences of FBXW8-encoding nucleic acids, and the proteins encoded by those nucleic acids, have been determined and deposited in a publicly available database (e.g., NCBI's Genbank database). In an embodiment of particular interest, the the FBXW8 protein has the amino acid sequence encoded by the nucleic acid sequence disclosed by NCBI GID: 26259. Other FBXW8 sequences deposited in NCBI's Genbank database include: GID:30795122 (accession number NM_{—}153348.2; Homo sapiens F-box and WD-40 domain protein 8 (FBXW8), transcript variant 1, mRNA); and GID: 30795120 (accession number NM_{—}012174.1; Homo sapiens F-box and WD-40 domain protein 8 (FBXW8), transcript variant 2, mRNA); GID: 34190635 (accession number BC037296.2; Homo sapiens F-box and WD-40 domain protein 8, transcript variant 1, mRNA); GID:70999265 (Accession no.: XM_{—}749259.1; Mus musculus (house mouse) chromosome 5 genomic contig, strain C57BL/6J); GID: 23272281 (accession no.: BC024091.1; Mus musculus F-box and WD-40 domain protein 8, mRNA), GID: 89036563 (accession no.: NW_{—}925395.1; Homo sapiens Homo sapiens chromosome 12 genomic contig, alternate assembly (based on Celera assembly); GID: 82899024 (accession no. NW_{—}001030796.1; Mus musculus chromosome 5 genomic contig, alternate assembly); GID: 89035772 (accession no.: NT_{—}009775.16; Homo sapiens chromosome 12 genomic contig, reference assembly); GID: 62658972 (accession no.: XM_{—}222223.3; Rattus norvegicus F-box and WD-40 domain protein 8); GID: 84139102 (accession no.: CX062960.1; Sus scrofa Porcine testis EST project); GID: 30795120 (Accession no.: NM_{—}012174.1; Pan troglodytes FBXW8 gene, VIRTUAL TRANSCRIPT, partial sequence, genomic survey sequence). The above Genbank accessions are incorporated by reference in their entirety, including the nucleic acid and protein sequences therein, and the annotation of those sequences, as of the earliest filing date of this patent application.

In certain embodiments, a FBXW8-encoding nucleic acid may have: a) at least 70% (e.g., at least 80%, at least 90%, at least 95%, at least 97% or at least 98% sequence identity) to a FBXW8 sequence deposited in NCBI's Genbank database; b) may hybridize under stringent conditions to a FBXW8 sequence deposited in NCBI's Genbank database; or c) may encode a polypeptide that has at least 70% (e.g., at least 80%, at least 90%, at least 93%, at least 95%, at least 97% or at least 98% sequence identity) to a FBXW8 sequence deposited in NCBI's Genbank database. Regions of nucleotide and amino acid sequences that are suitable for modification (e.g., by substitution, deletion, insertion, and/or addition) will be readily apparent to the ordinarily skilled artisan upon alignment of the above-referenced nucleic acid and/or amino acid sequences, where areas of conserved or shared sequence should generally be maintained.

MAPK (Mitogen-Activated Protein Kinase)

The inventors have demonstrated that MAPK specifically phosphorylates cyclin D1 at Thr286. Phosphorylation of cyclin D1 at Thr286 is required for FBXW8-mediated ubiquitination of cyclin D1 and degradation through the 26S proteasome pathway. In some embodiments, MAPK is provided with binding partners that can facilitate interaction of MAPK with cyclin D1 to mediated phosphorylation of cyclin D1. Such binding partners can be provided as isolated proteins, or can be provided as components in a cell extract, where the clel is one in which MAPK-mediated phosphorylation of cyclin D1 occurs (e.g., due to endogenous genes or recombinant modification). Because MAPK has been extensively studied, one of skill in the art would recognize that MAPK may be prepared according to any general method known in the art. Exemplary methods are specifically described below.

DNA sequences of MAPK genes and the proteins encoded by those genes have been determined and deposited in a publicly available database (e.g., NCBI's Genbank database). In an embodiment of particular interest, the MAPK protein has the amino acid sequence encoded by the nucleic acid sequence disclosed by NCBI GID:5594. Other MAPK sequences deposited in NCBI's Genbank database include: GID: 75709178 (accession number NM_{—}002745.4; Homo sapiens mitogen-activated protein kinase 1 (MAPK1), transcript variant 1, mRNA); GID: 75709179 (Accession no.: NM_{—}138957.2; Homo sapiens mitogen-activated protein kinase 1 (MAPK1), transcript variant 2, mRNA); GID: 84579908 (accession no.: NM_{—}001038663.1; Mus musculus mitogen activated protein kinase 1 (Mapk1), GID: 17389605 (accession no.: BC017832.1; Homo sapiens mitogen-activated protein kinase 1, transcript variant 2, mRNA; GID: 74000585 (accession no. XM_{—}861228.1; Canis familiaris (dog) similar to Dual specificity mitogen-activated protein kinase kinase 1 (MAP kinase kinase 1) (MAPKK 1) (ERK activator kinase 1) (MAPK/ERK kinase 1) (MEK1), transcript variant 6 (LOC478347), mRNA); GID: 55650216 (accession no.: XM_{—}512987.1; Pan troglodytes (chimpanzee) mitogen-activated protein kinase kinase 2; mitogen-activated protein kinase kinase 2, p45; MAP kinase kinase 2; MAPK/ERK kinase 2; dual specificitymitogen-activated protein kinase kinase 2). The above Genbank accessions are incorporated by reference in their entirety, including the nucleic acid and protein sequences therein, and the annotation of those sequences, as of the earliest filing date of this patent application.

In certain embodiments, a MAPK gene may have: a) at least 70% (e.g., at least 80%, at least 90%, at least 95%, at least 97% or at least 98% sequence identity) to a MAPK sequence deposited in NCBI's Genbank database; b) may hybridize under stringent conditions to a MAPK sequence deposited in NCBI's Genbank database; or c) may encode a polypeptide that has at least 70% (e.g., at least 80%, at least 90%, at least 93%, at least 95%, at least 97% or at least 98% sequence identity) to a MAPK sequence deposited in NCBI's Genbank database. Regions of nucleotide and amino acid sequences that are suitable for modification (e.g., by substitution, deletion, insertion, and/or addition) will be readily apparent to the ordinarily skilled artisan upon alignment of the above-referenced nucleic acid and/or amino acid sequences, where areas of conserved or shared sequence should generally be maintained (e.g., motifs, domains, and the like).

CULLIN 1 (CUL1)

CUL1 associates with SKP1 and FBXW8 to form a specific (SKP1-CUL7-FBXW8) E3 ligase complex which promotes the ubiquitination of phosphorylated cyclin D1. CUL1 is known in the art; thus one of ordinarly skill in the art would recognize that CUL1 may be prepared according to any any general method known in the art. Exemplary methods are specifically described below.

The DNA sequences of several CUL1 genesand the proteins encoded by those genes have been determined and deposited into NCBI's Genbank database. In an embodiment of particular interest, the CUL1 protein is encoded by the nucleic acid sequence disclosed by NCBI GID: 8454. Other CUL1 sequences deposited in NCBI's Genbank database include: GID: 32307160 (accession number NM_{—}003592.2; Homo sapiens cullin 1 (CUL1), mRNA); GID: 34328459 (Accession no.: NM_{—}012042.3; Mus musculus cullin 1 (Cul1), mRNA); GID: 3139076 (accession no.: AF062536.1; Homo sapiens cullin 1 mRNA, complete cds), GID: 5815402 (accession no.: AF176910.1; Mus musculus cullin 1 (Cul1) mRNA, complete cds, Mrna); GID: 42564211 (accession no. AY528252.1; Bos taurus (cattle) cullin 1 mRNA, partial cds); GID: 55733335 (accession no.: CR861282.1; Pongo pygmaeus (orangutan) Pongo pygmaeus mRNA; cDNA DKFZp4591053); GID: 50364553 (accession no.: AACC02000041.1; chromosome 7 Contg41, whole genome shotgun sequence). The above Genbank accessions are incorporated by reference in their entirety, including the nucleic acid and protein sequences therein, and the annotation of those sequences, as of the earliest filing date of this patent application.

In certain embodiments, a CUL1 gene may have: a) at least 70% (e.g., at least 80%, at least 90%, at least 95%, at least 97% or at least 98% sequence identity) to a CUL1 sequence deposited in NCBI's Genbank database; b) may hybridize under stringent conditions to a CUL1 sequence deposited in NCBI's Genbank database; or c) may encode a polypeptide that has at least 70% (e.g., at least 80%, at least 90%, at least 93%, at least 95%, at least 97% or at least 98% sequence identity) to a CUL1 sequence deposited in NCBI's Genbank database. Regions of nucleotide and amino acid sequences that are suitable for modification (e.g., by substitution, deletion, insertion, and/or addition) will be readily apparent to the ordinarily skilled artisan upon alignment of the above-referenced nucleic acid and/or amino acid sequences, where areas of conserved or shared sequence should generally be maintained (e.g., motifs, domains, and the like).

Cullin 7 (CUL7)

CUL7 associates with SKP1 and FBXW8 to form a specific (SKP1-CUL7-FBXW8) E3 ligase complex which promotes the ubiquitination of phosphorylated cyclin D1. CUL7 is known in the art, and thus the ordinarily skilled artisan would recognize that CUL7 may be prepared according any general method known in the art. Exemplary methods are specifically described below.

The DNA sequences of several CUL7 genes and the proteins encoded by those genes have been determined and deposited into NCBI's Genbank database. In an embodiment of particular interest, the CUL7 protein has the amino acid sequence encoded by the nucleic acid sequence disclosed in NCBI GID: 9820. Other CUL7 sequences deposited in NCBI's Genbank database include: GID: 21707140 (accession number AAH33647.1; Homo sapiens Cullin-7); GID: 18043940 (Accession no.: BC019645.1; Mus musculus cullin 7, mRNA); GID: 41872645 (accession no.: NM_{—}014780.3; Homo sapiens cullin 7 (CUL7), mRNA), GID: 58761521 (accession no.: NM_{—}025611.5; Mus musculus cullin 7 (Cul7), mRNA); GID: 55727518 (accession no. CAH90514.1; Pongo pygmaeus (orangutan) hypothetical protein); GID: 55727517 (accession no.: CR858277.1; Pongo pygmaeus (orangutan) cyclin D1 (mRNA; cDNA DKFZp469G0910); GID: 21707139 (accession no.: BC033647.1; Homo sapiens cullin 7, mRNA). The above Genbank accessions are incorporated by reference in their entirety, including the nucleic acid and protein sequences therein, and the annotation of those sequences, as of the earliest filing date of this patent application.

In certain embodiments, a CUL7 gene may have: a) at least 70% (e.g., at least 80%, at least 90%, at least 95%, at least 97% or at least 98% sequence identity) to a CUL7 sequence deposited in NCBI's Genbank database; b) may hybridize under stringent conditions to a CUL7 sequence deposited in NCBI's Genbank database; or c) may encode a polypeptide that has at least 70% (e.g., at least 80%, at least 90%, at least 93%, at least 95%, at least 97% or at least 98% sequence identity) to a CUL7 sequence deposited in NCBI's Genbank database. Regions of nucleotide and amino acid sequences that are suitable for modification (e.g., by substitution, deletion, insertion, and/or addition) will be readily apparent to the ordinarily skilled artisan upon alignment of the above-referenced nucleic acid and/or amino acid sequences, where areas of conserved or shared sequence should generally be maintained (e.g., motifs, domains, and the like).

SKP1 (S-Phase Kinase-associated Protein 1)

S-phase Kinase-associated Protein 1 (SKP1) (also known as SKP1a, SKP1b, Cyclin A/CDK2-associated protein p19, EMC19, MGC34403, OCP2, OCP-2, OCP-II, OCP-II protein, Organ of Corti protein 2, p19A, p19skp1, RNA polymerase II elongation factor-like protein, SIII, TCEB1L, and Transcription elongation factor B) associates with FBXW8 and either CUL1 or CUL7 to form E3 ligase complexes (SKP1-CUL1-FBXW8 or SKP1-CUL7-FBXW8) which promotes the ubiquitination of phosphorylated cyclin D1.

SKP1 is known in the art; thus one of skill in the art would recognize that SKP1 may be prepared according to any general method known in the art. Exemplary methods are specifically described below.

In an embodiment of particular interest, the SKP1 protein has an amino acid sequence encoded by the nucleic acid sequence disclosed by NCBI GID: 6500. Other exemplary SKP1 genes and the proteins encoded by those genes have been determined and deposited into NCBI's Genbank database. SKP1 sequences deposited in NCBI's Genbank database include: GID: GI:25777713 (accession number NP_{—}733779, Homo sapiens S-phase kinase-associated protein 1A isoform b); GID: 25777711 (accession number NP_{—}008861 Homo sapiens S-phase kinase-associated protein 1A isoform a); GID: 25777712 (accession no. NM_{—}170679.1, Homo sapiens S-phase kinase-associated protein 1A (p19A) (SKP1A) transcript variant 2); GID:25777710 (accession no. NM_{—}006930.2; Homo sapiens S-phase kinase-associated protein 1A (p19A) (SKP1A), transcript variant 1); GID: 31560542 (accession no. NM_{—}011543, Mus musculus S-phase kinase-associated protein 1A (Skp1a)); GID:31560543 (accession no. NP_{—}035673, Mus musculus S-phase kinase-associated protein 1A (Skp1a)). The above Genbank accessions are incorporated by reference in their entirety, including the nucleic acid and protein sequences therein, and the annotation of those sequences, as of the earliest filing date of this patent application.

In certain embodiments, an SKP1-encoding nucleic acid may have: a) at least 70% (e.g., at least 80%, at least 90%, at least 95%, at least 97% or at least 98% sequence identity) to a SKP1 sequence deposited in NCBI's Genbank database; b) may hybridize under stringent conditions to a SKP1 sequence deposited in NCBI's Genbank database; or c) may encode a polypeptide that has at least 70% (e.g., at least 80%, at least 90%, at least 93%, at least 95%, at least 97% or at least 98% sequence identity) to a SKP1 sequence deposited in NCBI's Genbank database. Regions of nucleotide and amino acid sequences that are suitable for modification (e.g., by substitution, deletion, insertion, and/or addition) will be readily apparent to the ordinarily skilled artisan upon alignment of the above-referenced nucleic acid and/or amino acid sequences, where areas of conserved or shared sequence should generally be maintained (e.g., motifs, domains, and the like).

Cyclin D1

Cyclin D1 contains a highly stringent (within 0.041 percentile) D-domain in amino acids 179-193 which is recognized by the Ras/Raf/MEK/ERK MAPK signaling cascade. MAPK specifically phosphorylates cyclin D1 at Thre-286 which is required for cyclin D1 to be polyubiquitinated and degraded through the 26S proteasome pathway. Because cyclin D1 has been extensively studied, one of skill in the art would recognize that cyclin D1 may be prepared according to any general method known in the art. Exemplary methods are specifically described below.

In an embodiment of particular interest the cyclin D1 protein has an amino acid sequence encoded by the nucleic acid sequence disclosed by NCBI GID:595. The DNA sequences of several cyclin D1 genes and the proteins encoded by those genes have been determined and deposited into NCBI's Genbank database. Other cyclin D1 sequences deposited in NCBI's Genbank database include: GID:16950654 (Accession number NM_{—}053056.1; Homo sapiens cyclin D1 (PRAD1: parathyroid adenomatosis 1) (CCND1 mRNA); GID: 16950655 (Accession number NP_{—}444284.1; cyclin D1 Homo sapiens); GID: 61368366 (accession number AY891237.1; Homo sapiens Synthetic construct Homo sapiens clone FLH019447.01 L cyclin D1 (CCND1) mRNA, partial cds.); GID: 473122 (Accession no.: X75207.1 GI:; R. norvegicus CCND1 mRNA for cyclin D1.); GID: 77628152 (accession no.: NM_{—}053056.2; Homo sapiens cyclin D1 (CCND1), mRNA), GID: 6680867 (accession no.: NM_{—}007631.1; Mus musculus cyclin D1 (Ccnd1), mRNA; GID: 86438381 (accession no. BC112798.1; Bos taurus (cattle) similar to C1/S-specific cyclin D1 (PRAD1 oncogene) (BCL-1 oncogene), mRNA); GID: 31377522 (accession no.: NM_{—}171992.2; Rattus norvegicus cyclin D1 (Ccnd1), mRNA); GID: 33991562 (accession no.: BC023620.2; Homo sapiens cyclin D1, mRNA). The above Genbank accessions are incorporated by reference in their entirety, including the nucleic acid and protein sequences therein, and the annotation of those sequences, as of the earliest filing date of this patent application.

In certain embodiments, a cyclin D1 gene may have: a) at least 70% (e.g., at least 80%, at least 90%, at least 95%, at least 97% or at least 98% sequence identity) to a cyclin D1 sequence deposited in NCBI's Genbank database; b) may hybridize under stringent conditions to a cyclin D1 sequence deposited in NCBI's Genbank database; or c) may encode a polypeptide that has at least 70% (e.g., at least 80%, at least 90%, at least 93%, at least 95%, at least 97% or at least 98% sequence identity) to a cyclin D1 sequence deposited in NCBI's Genbank database. Regions of nucleotide and amino acid sequences that are suitable for modification (e.g., by substitution, deletion, insertion, and/or addition) will be readily apparent to the ordinarily skilled artisan upon alignment of the above-referenced nucleic acid and/or amino acid sequences, where areas of conserved or shared sequence should generally be maintained (e.g., motifs, domains, and the like).

Nucleic Acid Molecules, Polypeptide Production Methods, Expression Vectors, Fusion Proteins

FBXW8 polypeptides, MAPK polypeptides, cyclin D1 polypeptides, CUL1 polypeptides, CUL7 polypeptides and SKP1 polypeptides for use in the assays and complexes described herein can be produced according to methods known in the art.

Nucleic Acids

The disclosure provides nucleic acid compositions encoding the MAPK polypeptides, FBXW8 polypeptides, cyclin D1 polypeptides, CUL1 polypeptides, CUL7 polypeptides and SKP1 polypeptides described herein. Exemplary nucleic acid and amino acid sequences for each of these polypeptides are provided above.

Nucleic acid compositions of particular interest comprise a sequence of DNA having an open reading frame that encodes a protein of interest (e.g., MAPK, FBXW8, cyclin D1, CUL1, CUL7, SKP1) and is capable, under appropriate conditions, of being expressed as a protein according to the subject invention.

In general, nucleic acids encoding a polypeptide of interest may be present in an appropriate vector for extrachromosomal maintenance or for integration into a host genome, as described in greater detail below. Where the regions associated with biological activity of the polypeptide is known, the nucleic acid may encode all or part of the polypeptide, with the proviso that the polypeptide provides the desired biological activity (e.g., phosphorylation of cyclin D1, mediation of ubiquitination of phosphorylated cyclin D1, etc.).

The polynucleotides of interest and constructs containing such polynucleotides can be generated synthetically by a number of different protocols known to those of skill in the art. Appropriate polynucleotide constructs are purified using standard recombinant DNA techniques as described in, for example, Sambrook et al., Molecular Cloning. A Laboratory Manual, 2nd Ed., (1989) Cold Spring Harbor Press, Cold Spring Harbor, N.Y., and under current regulations described in United States Dept. of HHS, National Institute of Health (NIH) Guidelines for Recombinant DNA Research.

Mutant nucleic acids can be generated by random mutagenesis or targeted mutagenesis, using well-known techniques that are routine in the art. The regions of the sequence that tolerate modification (e.g., conservative or non-conservative substitution) can be identified both from the results of the funcational assays provided in the Examples below and/or by sequence alignment of isoforms and homologs of a sequence to be modified. The DNA sequence or protein product of such a mutation will usually be substantially similar to the sequences provided herein, e.g. will differ by at least one nucleotide or amino acid, respectively, and may differ by at least two but not more than about ten nucleotides or amino acids. The sequence changes may be substitutions, insertions, deletions, or a combination thereof. Deletions may further include larger changes, such as deletions of a domain or exon, e.g. of stretches of 10, 20, 50, 75, 100, 150 or more aa residues. Techniques for in vitro mutagenesis (e.g., site-specific mutation) of cloned genes are known. In general, nucleic acids encoding a polypeptide of interest may be present in an appropriate vector for extrachromosomal maintenance or for integration into a host genome, as described in greater detail below. Where the regions associated with biological activity of the polypeptide is known, the nucleic acid may encode all or part of the polypeptide, with the proviso that the polypeptide provides the desired biological activity (e.g., phosphorylation of cyclin D1, mediation of ubiquitination of phosphorylated cyclin D1, etc.).

The polynucleotides of interest and constructs containing such polynucleotides can be generated synthetically by a number of different protocols known to those of skill in the art. Appropriate polynucleotide constructs are purified using standard recombinant DNA techniques as described in, for example, Sambrook et al., Molecular Cloning: A Laboratory Manual, 2nd Ed., (1989) Cold Spring Harbor Press, Cold Spring Harbor, N.Y., and under current regulations described in United States Dept. of HHS, National Institute of Health (NIH) Guidelines for Recombinant DNA Research.

Vectors

In general, nucleic acids encoding a polypeptide of interest may be present in an appropriate vector for extrachromosomal maintenance or for integration into a host genome, as described in greater detail below. Where the regions associated with biological activity of the polypeptide is known, the nucleic acid may encode all or part of the polypeptide, with the proviso that the polypeptide provides the desired biological activity (e.g., phosphorylation of cyclin D1, mediation of ubiquitination of phosphorylated cyclin D1, etc.). The expression vector may be either self-replicating extrachromosomal vectors or vectors which integrate into a host genome.

Generally, these expression vectors include transcriptional and translational regulatory nucleic acid operably linked to the nucleic acid encoding the protein. The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. As another example, operably linked refers to DNA sequences linked so as to be contiguous, and, in the case of a secretory leader, contiguous and in reading frame. However, enhancers do not have to be contiguous. Linking is accomplished by ligation at convenient restriction sites. If such sites do not exist, the synthetic oligonucleotide adapters or linkers are used in accordance with conventional practice. The transcriptional and translational regulatory nucleic acid will generally be appropriate to the host cell used to express the protein; for example, transcriptional and translational regulatory nucleic acid sequences from Bacillus can be used to express the protein in Bacillus. Numerous types of appropriate expression vectors, and suitable regulatory sequences are known in the art for a variety of host cells.

In general, the transcriptional and translational regulatory sequences may include, but are not limited to, promoter sequences, ribosomal binding sites, transcriptional start and stop sequences, translational start and stop sequences, and enhancer or activator sequences. In one embodiment, the regulatory sequences include a promoter and transcriptional start and stop sequences.

Promoter sequences contemplated include constitutive promoters and inducible promoters. The promoters may be either naturally occurring promoters or hybrid promoters. Hybrid promoters, which combine elements of more than one promoter, are also known in the art, and are useful in the present invention.

In addition, the expression vector may comprise additional elements. For example, the expression vector may have two replication systems, thus allowing it to be maintained in two organisms, for example in mammalian or insect cells for expression and in a prokaryotic host for cloning and amplification. Furthermore, for integrating expression vectors, the expression vector contains at least one sequence homologous to the host cell genome, and preferably two homologous sequences which flank the expression construct. The integrating vector may be directed to a specific locus in the host cell by selecting the appropriate homologous sequence for inclusion in the vector. Constructs for integrating vectors are well known in the art.

In addition, in one embodiment, the expression vector contains a selectable marker gene to allow the selection of transformed host cells. Selection genes are well known in the art and will vary with the host cell used.

Viral and non-viral vectors may be prepared and used, including plasmids, which provide for replication of DNA of interest and/or expression in a host cell. The choice of vector will depend on the type of cell in which propagation is desired and the purpose of propagation. Certain vectors are useful for amplifying and making large amounts of the desired DNA sequence. Other vectors are suitable for expression in cells in culture. Still other vectors are suitable for transformation and expression in cells in a whole animal or person. The choice of appropriate vector is well within the skill of the art. Many such vectors are available commercially. To prepare the constructs, the partial or full-length polynucleotide is inserted into a vector typically by means of DNA ligase attachment to a cleaved restriction enzyme site in the vector.

Alternatively, the desired nucleotide sequence can be inserted by homologous recombination in a cell. Typically this is accomplished by attaching regions of homology to the vector on the flanks of the desired nucleotide sequence. Regions of homology are added by ligation of oligonucleotides, or by polymerase chain reaction using primers comprising both the region of homology and a portion of the desired nucleotide sequence, for example.

Also provided are expression cassettes or systems that find use in, among other applications, the synthesis of the subject proteins. For expression, the gene product encoded by a polynucleotide of the invention is expressed in any convenient expression system, including, for example, bacterial, yeast, insect, amphibian and mammalian systems. In the expression vector, a subject polynucleotide is linked to a regulatory sequence as appropriate to obtain the desired expression properties. These regulatory sequences can include promoters (attached either at the 5′ end of the sense strand or at the 3′ end of the antisense strand), enhancers, terminators, operators, repressors, and inducers. The promoters can be regulated or constitutive.

In some situations it may be desirable to use conditionally active promoters, such as tissue-specific or developmental stage-specific promoters. These are linked to the desired nucleotide sequence using the techniques described above for linkage to vectors. Any techniques known in the art can be used. In other words, the expression vector will provide a transcriptional and translational initiation region, which may be inducible or constitutive, where the coding region is operably linked under the transcriptional control of the transcriptional initiation region, and a transcriptional and translational termination region. These control regions may be native to the subject species from which the subject nucleic acid is obtained, or may be derived from exogenous sources.

Eukaryotic promoters suitable for use include, but are not limited to, the following: the promoter of the mouse metallothionein I gene sequence (Hamer et al., J. Mol. Appl. Gen. 1:273-288, 1982); the TK promoter of Herpes virus (McKnight, Cell 31:355-365, 1982); the SV40 early promoter (Benoist et al., Nature (London) 290:304-310, 1981); the yeast gall gene sequence promoter (Johnston et al., Proc. Natl. Acad. Sci. (USA) 79:6971-6975, 1982); Silver et al., Proc. Natl. Acad. Sci. (USA) 81:5951-59SS, 1984), the CMV promoter, the EF-1 promoter, Ecdysone-responsive promoter(s), tetracycline-responsive promoter, and the like.

Promoters may be constitutive or regulatable (e.g, inducible). Inducible promoter elements are DNA sequence elements that act in conjunction with promoters and may bind either repressors (e.g. lacO/LACIq repressor system in E. coli) or inducers (e.g. gal1/GAL4 inducer system in yeast). In such cases, transcription is virtually “shut off” until the promoter is derepressed or induced, at which point transcription is “turned-on.”

Expression vectors generally have convenient restriction sites located near the promoter sequence to provide for the insertion of nucleic acid sequences encoding heterologous proteins. A selectable marker operative in the expression host may be present. Expression vectors may be used for, among other things, the screening methods described in greater detail below.

Expression cassettes may be prepared comprising a transcription initiation region, the gene or fragment thereof, and a transcriptional termination region. After introduction of the DNA, the cells containing the construct may be selected by means of a selectable marker, the cells expanded and then used for expression.

The above described expression systems may be employed with prokaryotes or eukaryotes in accordance with conventional ways, depending upon the purpose for expression. For large scale production of the protein, a unicellular organism, such as E. coli, B. subtilis, S. cerevisiae, insect cells in combination with baculovirus vectors, or cells of a higher organism such as vertebrates, e.g. COS 7 cells, HEK 293, CHO, Xenopus Oocytes, etc., may be used as the expression host cells. In some situations, it is desirable to express the gene in eukaryotic cells, where the expressed protein will benefit from native folding and post-translational modifications.

Specific expression systems of interest include bacterial, yeast, insect cell and mammalian cell derived expression systems. Expression vectors for bacteria are well known in the art, and include vectors for Bacillus subtilis, E. coli, Streptococcus cremoris, and Streptococcus lividans, among others. The bacterial expression vectors are transformed into bacterial host cells using techniques well known in the art, such as calcium chloride treatment, electroporation, and others.

Where expression in a bacterial host cell is desired (e.g., for polypeptide production), a suitable bacterial promoter is included in the vector, any nucleic acid sequence capable of binding bacterial RNA polymerase and initiating the downstream (3′) transcription of the coding sequence of a protein into mRNA. Sequences encoding metabolic pathway enzymes provide particularly useful promoter sequences. Examples include promoter sequences derived from sugar metabolizing enzymes, such as galactose, lactose and maltose, and sequences derived from biosynthetic enzymes such as tryptophan. Promoters from bacteriophage may also be used and are known in the art. In addition, synthetic promoters and hybrid promoters are also useful; for example, the tac promoter is a hybrid of the trp and lac promoter sequences. Furthermore, a bacterial promoter can include naturally occurring promoters of non-bacterial origin that have the ability to bind bacterial RNA polymerase and initiate transcription.

In addition to a promoter sequence, an efficient ribosome binding site is desirable. In E. coli, the ribosome binding site is called the Shine-Delgarno (SD) sequence and includes an initiation codon and a sequence 3-9 nucleotides in length located 3-11 nucleotides upstream of the initiation codon. Bacterial expression vectors may also include a signal peptide sequence that provides for secretion of the protein in bacteria. The signal sequence typically encodes a signal peptide comprised of hydrophobic amino acids which direct the secretion of the protein from the cell, as is well known in the art. The protein is either secreted into the growth media (gram-positive bacteria) or into the periplasmic space, located between the inner and outer membrane of the cell (gram-negative bacteria).

In one embodiment, proteins are produced in insect cells. Expression vectors for the transformation of insect cells, and in particular, baculovirus-based expression vectors, are well known in the art. In another embodiment, proteins are produced in yeast cells. Yeast expression systems are well known in the art, and include expression vectors for Saccharomyces cerevisiae, Candida albicans and C. maltosa, Hansenula polymorpha, Kluyveromyces fragilis and K. lactis, Pichia guillerimondii P. methanolica and P. pastoris, Schizosaccharomyces pombe, and Yarrowia lipolytica. Promoter sequences for expression in yeast include the inducible GAL1,10 promoter, the promoters from alcohol dehydrogenase, enolase, glucokinase, glucose-6-phosphate isomerase, glyceraldehyde-3-phosphate-dehydrogenase, hexokinase, phosphofructokinase, 3-phosphoglycerate mutase, pyruvate kinase, and the acid phosphatase gene. Yeast selectable markers include ADE2, HIS4, LEU2, TW1, and ALG7, which confers resistance to tunicamycin; the neomycin phosphotransferase gene, which confers resistance to G4 18; and the CUP1 gene, which allows yeast to grow in the presence of copper ions.

Mammalian expression can be accomplished as described in Dijkema et al., EMBO J. (1985) 4:761, Gorman et al., Proc. Natl. Acad. Sci. (USA) (1982) 79:6777, Boshart et al., Cell (1985) 41:521 and U.S. Pat. No. 4,399,216. Other features of mammalian expression are facilitated as described in Ham and Wallace, Meth. Enz. (1979) 58:44, Barnes and Sato, Anal. Biochem. (1980) 102:255, U.S. Pat. Nos. 4,767,704, 4,657,866, 4,927,762, 4,560,655, WO 90/103430, WO 87/00195, and U.S. RE Pat. No. 30,985.

Methods of introducing exogenous nucleic acid into mammalian hosts, as well as other hosts, is well known in the art, and will vary with the host cell used. Techniques include dextran-mediated transfection, calcium phosphate precipitation, polybrene mediated transfection, protoplast fusion, electroporation, viral infection, encapsulation of the polynucleotide(s) in liposomes, and direct microinjection of the DNA into nuclei.

Protein Production Methods

Proteins can be produced by culturing a host cell transformed with an expression vector containing nucleic acid encoding the protein, under the appropriate conditions to induce or cause expression of the protein. The conditions appropriate for protein expression will vary with the choice of the expression vector and the host cell, and will be easily ascertained by one skilled in the art through routine experimentation. For example, the use of constitutive promoters in the expression vector will require optimizing the growth and proliferation of the host cell, while the use of an inducible promoter requires the appropriate growth conditions for induction.

In a one embodiment, the proteins are expressed in mammalian cells, especially human cells, with cancerous cells, particularly human cancerous cells, being of interest. Mammalian expression systems are also known in the art, and include retroviral systems. A mammalian promoter (i.e., a promoter functional in a mammalian cell) is any DNA sequence capable of binding mammalian RNA polymerase and initiating the downstream (3′) transcription of a coding sequence for a protein into mRNA. A promoter will have a transcription initiating region, which is usually placed proximal to the 5′ end of the coding sequence, and a TATA box, using a located 25-30 base pairs upstream of the transcription initiation site. The TATA box is thought to direct RNA polymerase II to begin RNA synthesis at the correct site. A mammalian promoter will also contain an upstream promoter element (enhancer element), typically located within 100 to 200 base pairs upstream of the TATA box. An upstream promoter element determines the rate at which transcription is initiated and can act in either orientation. Of particular use as mammalian promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter.

The protein may also be made as a fusion protein, using techniques well known in the art. Thus, for example, the protein may be made fusion nucleic acid encoding the peptide or may be linked to other nucleic acid for expression purposes. Similarly, proteins of the invention can be linked to tags that are protein labels, such as an immunodetectable label (e.g., FLAG), a enzymatically detectable label (e.g., GST), and/or an optically detectable label (e.g., green fluorescent protein (GFP), red fluorescent protein (RFP), blue fluorescent protein (BFP), yellow fluorescent protein (YFP), luciferase, etc.)

Proteins may be isolated or purified in a variety of ways known to those skilled in the art depending on what other components are present in the sample. Standard purification methods include electrophoretic, molecular, immunological and chromatographic techniques, including ion exchange, hydrophobic, affinity, and reverse-phase HPLC chromatography, and chromatofocusing. For example, the ubiquitinated cyclin D1 may be isolated using a standard anti-ubiquitin antibody column. Phosphorylated cyclin D1 may be isolatd using an antibody specific for phosphorylated cyclin D1. Ultrafiltration and diafiltration techniques, in conjunction with protein concentration, are also useful. For general guidance in suitable purification techniques, see Scopes, R., Protein Purification, Springer-Verlag, NY (1982). The degree of purification necessary will vary depending on the use of the protein. In some instances no purification will be necessary.

Covalently Modified Proteins

MAPK polypeptides, FBXW8 polypeptides, cyclin D1 polypeptides, CUL1 polypeptides, CUL7 polypeptides, and SKP1 polypeptides having covalent modifications, particularly those that confer a feature useful in a screening assay as described below, are also provided herein. Of particular interest are polypeptides modified so as to incorporate a detectable tag.

Detectably Tagged Polypeptides

Polypeptides modified to comprises a tag and useful in the screening methods of the invention are specifically contemplated herein. By “tag” is meant an attached molecule or molecules useful for the identification or isolation of the attached molecule(s), which can be substrate binding molecules. For example, a tag can be an attachment tag or a label tag. Components having a tag are referred to as “tag-X”, wherein X is the component.

The terms “tag”, “detectable label” and “detetable tag” are used interchangeably herein without limitation. Usually, the tag is covalently bound to the attached component. By “tag”, “label”, “detectable label” or “detectable tag” is meant a molecule that can be directly (i.e., a primary label) or indirectly (i.e., a secondary label) detected; for example a label can be visualized and/or measured or otherwise identified so that its presence or absence can be known. As will be appreciated by those in the art, the manner in which this is performed will depend on the label. Exemplary labels include, but are not limited to, fluorescent labels (e.g. GFP) and label enzymes.

Exemplary tags include, but are not limited to, an optically-detectable label, a partner of a binding pair, and a surface substrate binding molecule (or attachment tag). As will be evident to the skilled artisan, many molecules may find use as more than one type of tag, depending upon how the tag is used. In one embodiment, the tag or label as described below is incorporated into the polypeptide as a fusion protein.

As will be appreciated by those in the art, tag-components of the invention can be made in various ways, depending largely upon the form of the tag. Components of the invention and tags are preferably attached by a covalent bond. Examples of tags are described below.

Exemplary Tags Useful in the Invention

In one embodiment, the tag is a polypeptide which is provided as a portion of a chimeric molecule comprising a first polypeptide fused to another, heterologous polypeptide or amino acid sequence. In one embodiment, such a chimeric molecule comprises a fusion of a first polypeptide with a tag polypeptide. The tag is generally placed at the amino-or carboxyl-terminus of the polypeptide. In embodiments in which the tagged polypeptide is to be used in a cell-based assay and is to be expressed a recombinant protein, the tag is usually a genetically encodable tag (e.g., fluorescent polypeptide, immunodetectable polypeptide, and the like).

The tag polypeptide can be, for example, an immunodetectable label (i.e., a polypeptide or other moiety which provides an epitope to which an anti-tag antibody can selectively bind), a polypeptide which serves as a ligand for binding to a receptor (e.g., to facilitate immobilization of the chimeric molecule on a substrate); an enzyme label (e.g., as described further below); or a fluorescent label (e.g., as described further below). Tag polypeptides provide for, for example, detection using an antibody against the tag polypeptide, and/or a ready means of isolating or purifying the tagged polypeptide (e.g., by affinity purification using an anti-tag antibody or another type of receptor-ligand matrix that binds to the tag). The production of tag-polypeptides by recombinant means is within the knowledge and skill in the art.

Production of immunodetectably-labeled proteins (e.g., use of FLAG, HIS, and the like, as a tag) is well known in the art and kits for such production are commercially available (for example, from Kodak and Sigma). See, e.g., Winston et al., Genes and Devel. 13:270-283 (1999), incorporated herein in its entirety, as well as product handbooks provided with the above-mentioned kits. Production of proteins having His-tags by recombinant means is well known, and kits for producing such proteins are commercially available. Such a kit and its use is described in the QIAexpress Handbook from Qiagen by Joanne Crowe et al., hereby expressly incorporated by reference.

Production of polypeptides having an optically-detectable label are well known. An “optically detectable label” includes labels that are detectably due to inherent properties (e.g., a fluorescent label), or which amy be reacted with a substrate or act as a substrate to provide an optically detectable (e.g., colored) reaction product (e.g., HRP).

By “fluorescent label” is meant any molecule that may be detected via its inherent fluorescent properties, which include fluorescence detectable upon excitation. Suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade BlueTM, Texas Red, IAEDANS, EDANS, BODIPY FL, LC Red 640, Cy 5, Cy 5.5, LC Red 705 and Oregon green. Suitable optical dyes are described in the 2002 Molecular Probes Handbook, 9th Ed., by Richard P. Haugland, hereby expressly incorporated by reference.

Suitable fluorescent labels include, but are not limited to, green fluorescent protein (GFP; Chalfie, et al., Science 263(5148):802-805 (Feb. 11, 1994); and EGFP; Clontech-Genbank Accession Number U55762), blue fluorescent protein (BFP; 1. Quantum Biotechnologies, Inc. 1801 de Maisonneuve Blvd. West, 8th Floor, Montreal (Quebec) Canada H3H 1J9; 2. Stauber, R. H. Biotechniques 24(3):462-471 (1998); 3. Heim, R. and Tsien, R. Y. Curr. Biol. 6:178-182 (1996)), enhanced yellow fluorescent protein (EYFP; 1. Clontech Laboratories, Inc., 1020 East Meadow Circle, Palo Alto, Calif. 94303), luciferase (Ichiki, et al., J. Immunol. 150(12):5408-5417 (1993)), -galactosidase (Nolan, et al., Proc Natl Acad Sci USA 85(8):2603-2607 (April 1988)) and Renilla WO 92/15673; WO 95/07463; WO 98/14605; WO 98/26277; WO 99/49019; U.S. Pat. No. 5,292,658; U.S. Pat. No. 5,418,155; U.S. Pat. No. 5,683,888; U.S. Pat. No. 5,741,668; U.S. Pat. No. 5,777,079; U.S. Pat. No. 5,804,387; U.S. Pat. No. 5,874,304; U.S. Pat. No. 5,876,995; and U.S. Pat. No. 5,925,558), and Ptilosarcus green fluorescent proteins (pGFP) (see WO 99/49019). All of the above-cited references are expressly incorporated herein by reference.

In some instances, multiple fluorescent labels are employed. In one embodiment, at least two fluorescent labels are used which are members of a fluorescence resonance energy transfer (FRET) pair. FRET can be used to detect association/dissociation of for example, MAPK and cyclin D1, FBXW8 and phosphorylated cyclin D1.; and the like. In general, such FRET pairs are used in in vitro assays.

FRET is phenomenon known in the art wherein excitation of one fluorescent dye is transferred to another without emission of a photon. A FRET pair consists of a donor fluorophore and an acceptor fluorophore (where the acceptor fluorophore may be a quencher molecule). The fluorescence emission spectrum of the donor and the fluorescence absorption spectrum of the acceptor must overlap, and the two molecules must be in close proximity. The distance between donor and acceptor at which 50% of donors are deactivated (transfer energy to the acceptor) is defined by the Forster radius, which is typically 10-100 angstroms. Changes in the fluorescence emission spectrum comprising FRET pairs can be detected, indicating changes in the number of that are in close proximity (i.e., within 100 angstroms of each other). This will typically result from the binding or dissociation of two molecules, one of which is labeled with a FRET donor and the other of which is labeled with a FRET acceptor, wherein such binding brings the FRET pair in close proximity.

Binding of such molecules will result in an increased fluorescence emission of the acceptor and/or quenching of the fluorescence 15 emission of the donor. FRET pairs (donor/acceptor) useful in the invention include, but are not limited to, EDANS/fluorescien, IAEDANS/fluorescein, fluoresceidtetramethylrhodamhe, fluoresceidLC Red 640, fluoresceidcy 5, fluoresceidcy 5.5 and fluoresceidLC Red.

In another aspect of FRET, a fluorescent donor molecule and a nonfluorescent acceptor molecule (“quencher”) may be employed. In this application, fluorescent emission of the donor will increase when quencher is displaced from close proximity to the donor and fluorescent emission will decrease when the quencher is brought into close proximity to the donor. Useful quenchers include, but are not limited to, DABCYL, QSY 7 and QSY 33. Useful fluorescent donodquencher pairs include, but are not limited to EDANS/DABCYL, Texas RedLDABCYL, BODIPYDABCYL, Lucifer yellowDABCYL, coumarin/DABCYL and fluoresceidQSY 7 dye.

The skilled artisan will appreciate that FRET and fluorescence quenching allow for monitoring of binding of labeled molecules over time, providing continuous information regarding the time course of binding reactions. It is important to remember that attachment of labels or other tags should not interfere with active groups on the interacting polypeptides. Amino acids or other moieties may be added to the sequence of a protein, through means well known in the art and described herein, for the express purpose of providing a linker and/or point of attachment for a label. In one embodiment, one or more amino acids are added to the sequence of a component for attaching a tag thereto, with a fluorescent label being of particular interest.

In other embodiments, detection involves bioluminescence resonance energy transfer (BRET). BRET is a protein-protein interaction assay based on energy transfer from a bioluminescent donor to a fluorescent acceptor protein. The BRET signal is measured by the amount of light emitted by the acceptor to the amount of light emitted by the donor. The ratio of these two values increases as the two proteins are brought into proximity. The BRET assay has been amply described in the literature. See, e.g., U.S. Pat. Nos. 6,020,192; 5,968,750; and 5,874,304; and Xu et al. (1999) Proc. Natl. Acad. Sci. USA 96:151-156. BRET assays may be performed by analyzing transfer between a bioluminescent donor protein and a fluorescent acceptor protein. Interaction between the donor and acceptor proteins can be monitored by a change in the ratio of light emitted by the bioluminescent and fluorescent proteins.

Alternatively, binding may be assayed by fluorescence anisotropy. Fluorescence anisotropy assays are amply described in the literature. See, e.g., Jameson and Sawyer (1995) Methods Enzymol. 246:283-300.

By “label enzyme” is meant an enzyme which may be reacted in the presence of a label enzyme substrate which produces a detectable product. Label enzymes may also be optically detectable labels (e.g., in the case of HRP), may Suitable label enzymes for use in the present invention include but are not limited to, horseradish peroxidase (HRP), alkaline phosphatase and glucose oxidase. Methods for the use of such substrates are well known in the art. The presence of the label enzyme is generally revealed through the enzyme's catalysis of a reaction with a label enzyme substrate, producing an identifiable product. Such products may be opaque, such as the reaction of horseradish peroxidase with tetramethyl benzedine, and may have a variety of colors. Other label enzyme substrates, such as Luminol (available fiom Pierce Chemical Co.), have been developed that produce fluorescent reaction products. Methods for identifying label enzymes with label enzyme substrates are well known in the art and many commercial kits are available. Examples and methods for the use of various label enzymes are described in Savage et al., Previews 247:6-9 (1998), Young, J. Virol. Methods 24:227-236 (1989), which are each hereby incorporated by reference in their entirety.

By “radioisotope” is meant any radioactive molecule. Suitable radioisotopes for use in the invention include, but are not limited to ¹⁴C, ³H, ³²P, ³³P, ³⁵S, ¹²⁵I, and ¹³¹I. The use of radioisotopes as labels is well known in the art.

In addition, labels may be indirectly detected, that is, the tag is a partner of a binding pair. By “partner of a binding pair” is meant one of a first and a second moiety, wherein said first and said second moiety have a specific binding affinity for each other. Suitable binding pairs for use in the invention include, but are not limited to, antigendantibodies (for example, digoxigeninlanti-digoxigenin, dinitrophenyl (DNP)/anti-DNP, dansyl-X-anti-dansyl, Fluoresceidanti-fluorescein, Lucifer yellow/anti-lucifer yellow, and rhodamine anti-rhodamine), biotirdavid (or biotirdstreptavidin) and calmodulin binding protein (CBP)/calmodulin. Other suitable binding pairs include polypeptides such as the FLAG-peptide (Hopp et al., BioTechnol, 6:1204-1210 (1988)); the KT3 epitope peptide (Martin et al., Science, 255:192-194 (1992)); tubulin epitope peptide (Skinner et al., J. Biol. Chem., 266: 15 163-15 166 (1991)); and the T7 gene 10 protein peptide tag (Lutz-Freyemuth et al., Proc. Natl. Acad. Sci. USA, a:6393-6397 (1990)) and the antibodies each thereto. Generally, in one embodiment, the smaller of the binding pair partners serves as the tag, as steric considerations in ubiquitin ligation may be important. As will be appreciated by those in the art, binding pair partners may be used in applications other than for labeling, such as immobilization of the protein on a substrate and other uses as described below.

As will be appreciated by those in the art, a partner of one binding pair may also be a partner of another binding pair. For example, an antigen (first moiety) may bind to a first antibody (second moiety) which may, in turn, be an antigen for a second antibody (third moiety). It will be further appreciated that such a circumstance allows indirect binding of a first moiety and a third moiety via an intermediary second moiety that is a binding pair partner to each. As will be appreciated by those in the art, a partner of a binding pair may comprise a label, as described above. It will further be appreciated that this allows for a tag to be indirectly labeled upon the binding of a binding partner comprising a label. Attaching a label to a tag which is a partner of a binding pair, as just described, is referred to herein as “indirect labeling”.

In one embodiment, the tag is surface substrate binding molecule. By “surface substrate binding molecule” and grammatical equivalents thereof is meant a molecule have binding affinity for a specific surface substrate, which substrate is generally a member of a binding pair applied, incorporated or otherwise attached to a surface. Suitable surface substrate binding molecules and their surface substrates include, but are not limited to poly-histidine (poly-his) or poly-histidine-glycine (poly-his-gly) tags and Nickel substrate; the Glutathione-S Transferase tag and its antibody substrate (available from Pierce Chemical); the flu HA tag polypeptide and its antibody 12CA5 substrate (Field et al., Mol. Cell. Biol., 8:2159-2165 (1988)); the c-myc tag and the 8F9,3C7,6E107 G4, B7 and 9E10 antibody substrates thereto (Evan et al., Molecular and Cellular Biol, 5:3610-3616 (1985)]; and the Herpes Simplex virus glycoprotein D (gD) tag and its antibody substrate (Paborsky et al., Protein Engineering, 3(6):547-553 (1990)). In general, surface binding substrate molecules useful in the present invention include, but are not limited to, polyhistidine structures (His-tags) that bind nickel substrates, antigens that bind to surface substrates comprising antibody, haptens that bind to avidin substrate (e.g., biotin) and CBP that binds to surface substrate comprising calmodulin.

Production of antibody-embedded substrates is well known; see Slinkin et al., Bioconj, Chem. 2:342-348 (1991); Torchilin et al., supra; Trubetskoy et al., Bioconi. Chem. 33323-327 (1992); King et al., Cancer Res. 54:6176-6185 (1994); and Wilbur et al., Bioconjugate Chem. 5:220-235 (1994) (all of which are hereby expressly incorporated by reference), and attachment of or production of proteins with antigens is described above. Calmodulin-embedded substrates are commercially available and production of proteins with CBP is described in Simcox et al., Strategies 8:40-43 (1995), which is hereby incorporated by reference in its entirety.

Where appropriate, functionalization of labels with chemically reactive groups such as thiols, amines, carboxyls, etc. is generally known in the art. In one embodiment, the tag is functionalized to facilitate covalent attachment.

Biotinylation of target molecules and substrates is well known, for example, a large number of biotinylation agents are known, including amine-reactive and thiol-reactive agents, for the biotinylation of proteins, nucleic acids, carbohydrates, carboxylic acids; see, e.g., chapter 4, Molecular Probes Catalog, Haugland, 6th Ed. 1996, hereby incorporated by reference. A biotinylated substrate can be attached to a biotinylated component via avidin or streptavidin. Similarly, a large number of haptenylation reagents are also known. Methods for labeling of proteins with radioisotopes are known in the art. For example, such methods are found in Ohta et al., Molec. Cell 3:535-541 (1999), which is hereby incorporated by reference in its entirety.

The covalent attachment of the tag may be either direct or via a linker. In one embodiment, the linker is a relatively short coupling moiety that is used to attach the molecules. A coupling moiety may be synthesized directly onto a component of the invention, ubiquitin for example, and contains at least one functional group to facilitate attachment of the tag. Alternatively, the coupling moiety may have at least two functional groups, which are used to attach a functionalized component to a functionalized tag, for example. In an additional embodiment, the linker is a polymer. In this embodiment, covalent attachment is accomplished either directly, or through the use of coupling moieties from the component or tag to the polymer.

In one embodiment, the covalent attachment is direct, that is, no linker is used. In this embodiment, the component can contain a functional group such as a carboxylic acid which is used for direct attachment to the functionalized tag. It should be understood that the component and tag may be attached in a variety of ways, including those listed above. What is important is that manner of attachment does not significantly alter the functionality of the component. For example, in tag-ubiquitin, the tag should be attached in such a manner as to allow the ubiquitin to be covalently bound to other ubiquitin to form polyubiquitin chains.

As will be appreciated by those in the art, the above description of covalent attachment of a label and ubiquitin applies equally to the attachment of virtually any two molecules of the present disclosure. In one embodiment, the tag is functionalized to facilitate covalent attachment, as is generally outlined above. Thus, a wide variety of tags are commercially available which contain functional groups, including, but not limited to, isothiocyanate groups, amino groups, haloacetyl groups, maleimides, succinimidyl esters, and sulfonyl halides, all of which may be used to covalently attach the tag to a second molecule, as is described herein. The choice of the functional group of the tag will depend on the site of attachment to either a linker, as outlined above or a component of the invention. Thus, for example, for direct linkage to a carboxylic acid group of FBXW8 F-box protein, amino modified or hydrazine modified tags will be used for coupling via carbodimide chemistry, for example using 1-ethyl-3-(3-dimethylaminopropyl)-carbodimide (EDAC) as is known in the art (see Set 9 and Set 11 of the Molecular Probes Catalog, supra; see also the Pierce 1994 Catalog and Handbook, pages T-155 to T-200, both of which are hereby incorporated by reference). In one embodiment, the carbodimide is first attached to the tag, such as is commercially available for many of the tags described herein.

Components for Polyubitquitination Assays

In some aspects, the assays of the invention involve assessing ubiquitination of phosphorylated cyclin D1 as mediated by an FBXW8-containing E3 ligase. Such assays can be conducted in vitro using isolated phosphorylated cyclin D1, isolated FBXW8, and cell extracts to provide other components of the ubiquitination pathway (e.g., SKP1 and at least one of CUL1 or CUL7, and the ubiquitin moiety). Alternatively, the minimal components required for ubiquitination of phosphorylated cyclin D1 are provided in solution under conditions suitable for ubiquitination of phosphorylated cyclin D1. The following describes the components of the ubiquitination pathway for phosphorylated cyclin D1.

In general, a ubiquitin pathway involves a ubiquitin moiety, an ubiquitin activating agent (E1), an ubiquitin conjugatin agent (E2), and a ubiquitin ligase (E3). In the assays of the present invention, the E3 comprises FBXW8 in a complex with SKP1 and at least one of CUL1 or CUL7. In general, ubiquitination assays are conducted with phosphorylated cyclin D1 (as the ubiquitin target substrate), a ubiquitin moiety, an E1, an E2, and the FBXW8-containing E3. Alternatively, the assays do not require an E1 or separate ubiquitin moiety, but instead involve a ubiquitinated E2. The components of the assay are provided below.

Ubiquitin Moieties

By “ubiquitin” or “ubiquitin moiety” is meant a polypeptide which is transferred or attached to another polypeptide by a ubiquitin agent. Ubiquitin as used in the assays below is generally selected to be a ubiquitin compatible for ubiquitination of phosphorylated cyclin D1 as mediated by FBXW8-containing E3 ligase. In an embodiment of particular interest, the ubiquitin moiety is encoded by as the nucleic acid sequence disclosed by GenBank accession number X04803.2.

As used herein, “poly-ubiquitin moiety” refers to a chain of ubiquitin moieties comprising more than one ubiquitin moiety. As used herein, “mono-ubiquitin moiety” refers to a single ubiquitin moiety. In the screening methods of the present invention, an un-ubiquitylated, phosphorylated cyclin D1 protein, or a mono- or poly-ubiquitylated, phosphorylated cyclin D1 protein can serve as a substrate for an FBXW8-containing E3 ligase for the transfer or attachment of a ubiquitin moiety (which can itself be a mono- or poly-ubiquitin moiety).

Variants of the ubiquitin moiety which retain characteristics of the native ubiquitin moiety in being capable of being attached and/or cleaved from a target substrate protein. Such ubiquitin moiety variants generally have an overall amino acid sequence identity of preferably greater than about 75%, more preferably greater than about 80%, even more preferably greater than about 85% and most preferably greater than 90% of the amino acid sequence of ubiquitin provided above. In some embodiments the sequence identity will be as high as about 93 to 95 or 98%. Regions of nucleotide and amino acid sequences that are suitable for modification (e.g., by substitution, deletion, insertion, and/or addition) will be readily apparent to the ordinarily skilled artisan upon alignment of the above-referenced nucleic acid and/or amino acid sequences, where areas of conserved or shared sequence should generally be maintained (e.g., domains, motifs).

Ubiquitin moieties useful in the assays may be shorter or longer than the amino acid sequence of human ubiquitin depicted above. For example, ubiquitin moieties can be made longer than the reference amino acid sequence; for example, by the addition of tags, the addition of other fusion sequences, or the elucidation of additional coding and non-coding sequences. As described below, the fusion of a ubiquitin moiety to a fluorescent peptide, such as Green Fluorescent Peptide (GFP), is of particular interest.

In one embodiment where the assay is conducated in a cell, the ubiquitin moiety can be endogenous (i.e., naturally expressed in the cell to be assayed). In an alternative embodiment, the ubiquitin moiety, as well as other proteins involved in the ubiquitination pathway, are exogenous, e.g., recombinant proteins.

Ubiquitin Activating Agents (E1)

As used herein “ubiquitin activating agent” or “E1” refers to a ubiquitin agent that transfers or attaches a ubiquitin moiety to a ubiquitin conjugating agent (E2). Generally, the ubiquitin activating agent forms a high energy thiolester bond with ubiquitin moiety, thereby “activating” the ubiquitin moiety, and transfers or attaches the ubiquitin moiety to a ubiquitin conjugating agent (e.g., E2).The ubiquitin activating agent is an E1, which can bind ubiquitin and transfer or attach ubiquitin to an E2, defined below.

In generally, the E1 is Ubiquitin Activating Enzyme having the amino acid sequence disclosed by GenBank Protein accession number NP_{—}003325, incorporated herein by reference. Ubiquitin Activating Enzyme is also described in Handley et al. 1991. Proc Natl Acad Sci USA, 88 (1), 258-262; and Handley et al. 1991. Proc Natl Acad Sci USA, Proc Natl Acad Sci USA, 88 (16), 7456; herein incorporated by reference. Human recombinant E1 is commercially available from BostonBiochem (Cat. # E-305). E1-encoding nucleic acids which may be used for producing E1 proteins for the invention include, but are not limited to, those disclosed by GenBank accession number M58028 and X56976, incorporated herein by reference.

The invention also contemplates use of variants of E1 which retain a characteristic of a native ubiquitin activating agent in being capable of facilitating activation of a ubiquitin conjugating agent. Such ubiquitin activating agent variants generally have an overall amino acid sequence identity of preferably greater than about 75%, more preferably greater than about 80%, even more preferably greater than about 85% and most preferably greater than 90% of the amino acid sequence of a ubiquitin provided above. In some embodiments the sequence identity will be as high as activating agent about 93% to 95% or 98%. Regions of nucleotide and amino acid sequences that are suitable for modification (e.g., by substitution, deletion, insertion, and/or addition) will be readily apparent to the ordinarily skilled artisan upon alignment of the above-referenced nucleic acid and/or amino acid sequences, where areas of conserved or shared sequence should generally be maintained.

Ubiquitin Conjugating Agents (E2)

As used herein “ubiquitin conjugating agent” or “E2” refers to a ubiquitin agent, capable of facilitating transfer or attaching a ubiquitin moiety to a substrate protein through interaction with a ubiquitin ligating agent. The ubiquitin conjugating agent generally facilitates transfer or attachment of a ubiquitin moiety to a mono- or poly-ubiquitin moiety, which in turn can be attached to a ubiquitin agent or target protein.

In general, the E2 used in the ubiquitination assays is UbcH5c, which is encoeed by the nucleic acid sequence disclosed by NCBI GID: 7323, herein incorporated by reference. UbcH5c can have the amino acid sequence disclosed in GenBank accession numbers: NP_{—}871616; AAA91461; NP_{—}871619; NP_{—}871622; NP_{—}871618; NP_{—}871615; NP_{—}003331; NP_{—}871617; NP_{—}871620; and NP_{—}871621; each of which are herein incorporated by reference. In embodiments of particular interest, E2 is a human E2. Human recombinant E2 is commercially available from BostonBiochem (Cat. # E2-627).

Sequences encoding a ubiquitin conjugating agent may also be used to make variants thereof that are suitable for use in the methods and compositions of the present invention. The ubiquitin conjugating agents and variants suitable for use in the methods and compositions of the present invention may be made as described herein.

The invention contemplates use of variants of E2 which retain a characteristic of a native ubiquitin conjugating agent in being capable of being activated by a ubiquitin activating agent and/or facilitating ubiquitylation of a target substrate protein in connection with a ubiquitin ligating agent. Such ubiquitin conjugating agent variants generally have an overall amino acid sequence identity of preferably greater than about 75%, more preferably greater than about 80%, even more preferably greater than about 85% and most preferably greater than 90% of the amino acid sequence of a ubiquitin conjugating agent provided above. In some embodiments the sequence identity will be as high as about 93 to 95 or 98%. Regions of nucleotide and amino acid sequences that are suitable for modification (e.g., by substitution, deletion, insertion, and/or addition) will be readily apparent to the ordinarily skilled artisan upon alignment of the above-referenced nucleic acid and/or amino acid sequences, where areas of conserved or shared sequence should generally be maintained. Variants include E2 having a tag, as defined herein, where the complex can be referred to as “tag-E2”. Exemplary E2 tags include, but are not limited to, labels, partners of binding pairs and substrate binding elements. In one embodiment of particular interest, the tag is a His-tag or GST-tag.

FBXW8-Containing Ubiquitin Ligating Agent (E3)

Ubiquitination assays of the invention involve a FBXW8-containg E3 as the ubiquitin ligating agent (E3). As used herein “ubiquitin ligating agent” refers to a ubiquitin agent, in this case a complex of proteins, which facilitates transfer or attachment of a ubiquitin moiety from a ubiquitin conjugating agent (E2) to phosphorylated cyclin D1. As dicussed herein, the FBXW8-containing E3 is composed of the partners FBXW8, SKP1, and at least one of CUL1 or CUL7. Components of the FBXW8-containing E3 have been described in detail above. The E3 complex can be formed by combining the complex partners in vitro or in vivo (e.g., in a cell that expresses all or some of the components from an endogenous gene or form an exogenous (recombinant) gene). Where the complex is formed in vitro, complex partners can be provided as isolated proteins, or in cell extracts (where the extract is obtained form a cell in which FBXW8-medaited ubiquitination occurs).

Host Cells for Use in Assays

Cells suitable for use with the assay methods of the present invention are generally any higher eukaryotic cell in which cyclin D1 phosphorylation and ubiquitin-mediated degradation occurs, or which has been modified recombinantly to provide the necessary components. Usually the host cells in the assays are mammalian cells.

It will be desirable that the cells are an easily manipulated, easily cultured mammalian cell line, preferably human cell lines. In other embodiments, cells suitable for use are non-transformed primary human cells. In still other embodiments, cells suitable for use with subject invention are cells derived from a patient sample such as a cell biopsy, wherein the cells may or may not have distinct characteristics associated with a proliferative cellular disease associated with aberrant cyclin D1 phosphorylation and/or cyclin D1 degradation (e.g., due to over-expression of cyclin D1, aberrations in MAPK activity, aberrations in FBXW8 activity, and the like). Cancer cells and cell lines are of particular interest in the assays of the invention.

Exemplary cell lines for use as cells in assays include, but are not necessarily limited to, mammalian cell lines (particularly human cell lines). Specific exemplary cells include, but are not limited to, HCT 116, SW480, T98G, CCD841 CoN, WI-38, NIH 3T3, U-2 OS, and HEK293 cells, and the like.

In some embodiments, the cells used in the assay exhibit overexpression of cyclin D1 relative to a normal cell of the same tissue origin are of particular interest, such as cancerous cells (e.g., in screening for inhibitors of cellular proliferation). Exemplary cancer cells in which cyclin D1 overexpression has been implicated in tumorigeneis include, without limitation: breast cancer (e.g., carcinoma in situ (e.g., ductal carcinoma in situ), estrogen receptor (ER)-positive breast cancer, ER-negative breast cancer, breast cancers having a mutant BRCA1 allele or other forms and/or stages of breast cancer); lung cancer (e.g., small cell carcinoma, non-small cell carcinoma, mesothelioma, and other forms and/or stages of lung cancer); colon cancer (e.g., adenomatous polyp, colorectal carcinoma, and other forms and/or stages of colon cancer) ovarian cancer; endometrial cancer; oral cancers (e.g., oral squamous cell carcinomas) squamous cell carcinoma of the head and neck; liver cancer (e.g., hepatitis-related liver cancer); pancreatic cancer; esophageal carcinoma; laryngeal cancer; leukemias, lymphomas; neural cancers; and rhabdoid tumors. As noted above, the cancer cells can be cancer cell lines, primary cells isolated from a tumor, or cell lines generated from primary tumor cells.

Recombinant Cells

In several embodiments, the assays of the invention are conducted using host cells engineered to express or overexpress one or more of polypeptides involved in the cyclin D1 phosphorylation pathway (e.g., MAPK and/or cyclin D1) and/or one more polypeptides involved in the ubiquitin-mediated degradation of phosphorylated cyclin D1 (e.g., cyclin D1, FBXW8, CUL1, CUL7, SKP1). The recombinant polypeptides expressed in such recombinant cells can be modified to include a genetically encodable tag, as discussed above.

The cell line is most conveniently one that can be readily propagated in culture and is readily manipulated using recombinant techniques. The host cells used for production of such recombinant cells can be any cell discussed above, including cell lines, primary cells, and the like, including primary cancer cells and cancer cell lines. Exemplary cell lines, include, but are not necessarily limited to, mammalian cell lines (particularly human cell lines), such as HCT 116, SW480, T98G, CCD841 CoN, WI-38, NIH 3T3, U-2 OS, and HEK293 cells, and the like.

In general, the recombinant cells can be produced as described above. The constructs can be introduced into the host cell using standard methods practiced by one with skill in the art. Where one or more recombinant polypeptides are to be introduced into the cell as a polynucleotides encoding the one or more polypeptides and an expression cassette, optionally carried on one or more transient expression vectors (e.g., the vector is maintained in an episomal manner by the cell), which comprise the polynucleotides encoding the desired polypeptides. Alternatively, or in addition, the one or more expression constructs encoding one or more polypeptides can be stably integrated into the cell line. In addition or alternatively, one or more of polynucleotides encoding one or more desired polypeptides can be stably integrated into the cell, while one or more other desired polypeptides expressed from one or more transient expression vectors. For example, a polynucleotide encoding a cyclin D1 polypeptides may be stably integrated in the cell line, while a polynucleotide encoding a FBXW8 polypeptide, CUL1 (or CUL7), and SKP1 are expressed from one or more transient expression vectors. Likewise, a polynucleotide encoding MAPK polypeptide may be stably integrated in the cell line, while a polynucleotide encoding a detectably labeled cyclin D1 is expressed from a transient expression vector. Other variations and combinations of stably integrated vectors and transient expression vectors will be readily apparent to the skilled artisan upon reading the present disclosure.

Candidate Agents

The assays of the invention are designed to identify candidate agents that act as modulators of cyclin D1 phosphorylation and/or cyclin D1 ubiquitylation as mediated by MAPK and by FBXW8, respectively. By “modulator” is meant a compound which can facilitate an increase or decrease in at least one of cyclin D1 phosphorylation or cyclin D1 ubiquitylation. The skilled artisan will appreciate that modulators of cyclin D1 phosphorylation may, for example, affect activity MAPK, including activity in transfer or removal of phosphase group from Thr286 of cyclin D1, interaction between MAPK and cyclin D1, or a combination of these. Modulators of cyclin D1 ubiquitylation may affect activity of an FBXW8-containing E3 ligase, including activity in transfer or removal of a ubiquitin moiety to phosphorylated cyclin D1, interaction between the FBXW8-containing E3 ligase and phosphorylated cyclin D1, combination of these and/or other biological activities related to ubiquitylation.

By “test agent” or “candidate agent”, “candidate”, “candidate modulator”, “candidate ubiquitination modulator”, “candidate phosphorylation modulator” or grammatical equivalents herein, which terms are used interchangeably herein, is meant any molecule (e.g. proteins (which herein includes proteins, polypeptides, and peptides), small (i.e., 5-1000 Da, 100-750 Da, 200-500 Da, or less than 500 Da in size), or organic or inorganic molecules, polysaccharides, polynucleotides, etc.) which are to be tested for activity in modulating an activity associated with cellular proliferation and mediated through cyclin D1 (e.g., phosphorylation cyclin D1, or ubiquitination of cyclin D1).

A variety of different candidate agents may be screened by the above methods. Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Moreover, screening may be directed to known pharmacologically active compounds and chemical analogs thereof, or to new agents with unknown properties such as those created through rational drug design.

In one embodiment, candidate modulators are synthetic compounds. Any number of techniques are available for the random and directed synthesis of a wide variety of organic compounds and biomolecules, including expression of randomized oligonucleotides. See for example WO 94/24314, hereby expressly incorporated by reference, which discusses methods for generating new compounds, including random chemistry methods as well as enzymatic methods. As described in WO 94/24314, one of the advantages of the present method is that it is not necessary to characterize the candidate modulator prior to the assay; only candidate modulators that affect ubiquitylation of a target substrate protein of interest need be identified.

In another embodiment, the candidate modulators are provided as libraries of natural compounds in the form of bacterial, fungal, plant and animal extracts that are available or readily produced. Additionally, natural or synthetically produced libraries and compounds are readily modified through conventional chemical, physical and biochemical means. Known pharmacological agents may be subjected to directed or random chemical modifications, including enzymatic modifications, to produce structural analogs.

In one embodiment, candidate modulators include proteins (including antibodies, antibody fragments (i.e., a fragment containing an antigen-binding region, e.g., a FAb), single chain antibodies, and the like), nucleic acids, and chemical moieties. In one embodiment, the candidate modulators are naturally occurring proteins or fragments of naturally occurring proteins. Thus, for example, cellular extracts containing proteins, or random or directed digests of proteinaceous cellular extracts, may be tested, as is more fully described below. In this way libraries of procaryotic and eucaryotic proteins may be made for screening against any number of ubiquitin ligase compositions. Other embodiments include libraries of bacterial, fungal, viral, and mammalian proteins, with the latter being preferred, and human proteins being especially preferred.

In one embodiment, the candidate modulators are organic moieties. In this embodiment, as is generally described in WO 94/243 14, candidate agents are synthesized from a series of substrates that can be chemically modified. “Chemically modified” herein includes traditional chemical reactions as well as enzymatic reactions. These substrates generally include, but are not limited to, alkyl groups (including alkanes, alkenes, alkynes and heteroalkyl), aryl groups (including arenes and heteroaryl), alcohols, ethers, amines, aldehydes, ketones, acids, esters, amides, cyclic compounds, heterocyclic compounds (including purines, pyrimidines, benzodiazepins, beta-lactams, tetracylines, cephalosporins, and carbohydrates), steroids (including estrogens, androgens, cortisone, ecodysone, etc.), alkaloids (including ergots, vinca, curare, pyrollizdine, and mitomycines), organometallic compounds, hetero-atom bearing compounds, amino acids, and nucleosides. Chemical (including enzymatic) reactions may be done on the moieties to form new substrates or candidate agents which can then be tested using the present invention.

Assays to Identify Agents that Modulate Cell Proliferation Through Modulation of Ubiquitination of Cyclin D1 and/or Modulation of MAPK-mediated Cyclin D1 Phosphorylation

The invention provides methods for identifying agents that modulate cell proliferation. The screening methods may be designed a number of different ways, where a variety of assay configurations and protocols may be employed, as are known in the art. In general, the assay methods provide for identification of agents that modulate ubiquitination of phosphorylated cyclin D1 mediated by an FBXW8-containing E3 ligase, and for identification of agents that modulate activity of MAPK in phosphorylation of cyclin D1. It will be appreciated that the assays can be performed alone, in series or parallel, and in some instances can be performed in a single assay (e.g., MAPK-mediated cyclin D1 phosphorylation and FBXW8-mediated cyclin D1 ubiquitination can be assessed in the same assay).

It will be readily apparent to the ordinarily skilled artisan upon reading the present disclosure that appropriate positive and/or negative controls may be included in the inventive assays. Exemplary positive controls include an assay performed with an agnet which is known to modulate the parameter being tested (e.g., a parameter that is a direct or indirect result of FBXW8-mediated activity in ubiquitination of phosphorylated cyclin D1 and/or degradation of phosphorylated cyclin D1; and/oor a parameter that is a direct or indirect result of MAPK-mediated activity in phosphorylation of cyclin D1). Exemplary negative controls include an assay performed in the absence of a component essential for the activity (e.g,. FBXW8 or MAPK; cyclin D1; ubiquitination or a source of phosphate (e.g., ATP), and the like).

The assays can be used to identify test agents having a desired activity; to confirm activity of agents known to have activity in modulation of cellular proliferation, MAPK-mediated cyclin D1 phosphorylation, and/or FBXW8-mediated ubiquitination of phosphorylated cyclin D1; and/or as a counterscreens to identify agents that modulate FBXW8-mediated ubiquitination of phosphorylated cyclin D1 without substantially affecting MAPK activity or, alternatively to identify agents that modulate MAPK activity without substantially affecting FBXW8-mediated ubiquitination of phosphorylated cyclin D1.

Exemplary assay formats are provided below.

Identification of Agents that Modulate FBXW8-mediated Ubiquitination of Phosphorylated Cyclin D1 and/or Degradation of Phosphorylated Cyclin D1

In one aspect, the invention provides methods for identifying agents that modulate FBXW8 activity. FBXW8 forms an E3 ubiquitin ligase complex which specifically interacts with phosphorylated cyclin D1. The FBXW8-containing E3 ligase complex includes either CUL7 or CUL1 and SKP1.

The three proteins form an SCF-like complex which recognizes cyclin D1 in a phosphorylation-dependent manner to mediate ubiquitination of cyclin D1. It will be readily apparent to the skilled artisan upon reading the present disclosure that many of the assays may be performed in vitro (i.e., cell-free) or in vivo (i.e., in a cell).

In general, assays to identify agents that modulate ubiquitination of phosphorylated cyclin D1 by FBXW8 involve contacting a test agent with phosphorylated cyclin D1 and FBXW8 (which may be provided in a FBXW8-containing E3 ligase complex), wherein the phosphorylated cyclin D1 and FBXW8 may be present in a cell-free assay or within a cell. Where cells are used in the assay, the cell may be a cell recombinant for one or both of phosphorylated cyclin D1 and FBXW8. In either in vitro or cell-based assays, one or both of cyclin D1 and FBXW8 may be detectably labeled. If both are detectably labeled, then the labels are different so as to provide for signals that are distinguishable. The agent is contacted with the phosphorylated cyclin D1 and FBXW8 for a time sufficient for the interaction between phosphorylated cyclin D1 and FBXW8 to occur, and the effect of the agent detected. Effects on interaction of phosphorylated cyclin D1 and FBXW8 can be detected by detecting an effect on binding of phosphorylated cyclin D1 and FBXW8, or an effect on activity of FBXW8 in mediating ubiquitination and/or ubiquitin-mediated ubiquitination.

An agent that modulates (increases or decreases) FBXW8-phosphorylated cyclin D1 interactions (as detected directly (e.g., by detecting binding of FBXW8 and phosphorylated cyclin D1) or indirectly (e.g., by detecting ubiquitination of phosphorylated cyclin D1, levels of total or phosphorylated cyclin D1, and the like) is an agent that provides for a change of at least about 10%, at least about 20%, at least about 30%, at least about 50%, at least about 75%, at least about 100%, at least about 2.5-fold, at least about 3-fold, at least about 4-fold, at least about 5-fold, at least about 10-fold, at least about 20-fold, or at least about 50-fold, in the detected parameter associated with FBXW8-phosphorylated cyclin D1 interaction (e.g., binding; ubiquitinated phosphorylated cyclin D1, total cyclin D1 levels; phosphorylated cyclin D1 levels, and the like).

Assays Assessing FBXW8 Binding with Phosphorylated Cyclin D1

The screening methods provided herein include assays to identify an agent that modulates binding of FBXW8 with phosphorylated cyclin D1. Such assays can be conducted in vitro (e.g., in vitro binding assays) or in vivo (e.g, using cells having detectably labeled FBXW8, detectably labeled cyclin D1, or both). Exemplary assays are described below.

The assay can involve, for example, contacting phosphorylated cyclin D1 and FBXW8, which in such assays is provided as an FBXW8-containing E3 complex (i.e., gb-CUL1/7-SKP1) with a test agent, and directly determining the effect, if any, of the test agent on the binding of phosphorylated cyclin D1 and FBXW8 or FBXW8-containing E3 complex. This methods can be conducted in vitro (i.e., cell-free) in a reaction mixture, using isolated polypeptides. Where desired or required, the in vitro assay reaction mixture can comprise cell extracts (e.g., cell cytoplasm extracts) so as to provide cellular components required for interaction between FBXW8 and phosphorylated cyclin D1. The cell extractis prepared from a cell in which FBXW8-mediated ubiquitination of phosphorylated cyclin D1 occurs (e.g., due to endogenous activity or activity as a result of genetic modification). Alternatively, the assay can be performed in a cell-based assay, where the cell can provide for assay components by expression from an endogenous or non-endogenous (recombinant) nucleic acid.

Formation of a binding complex between phosphorylated cyclin D1 and FBXW8 can be detected using any known method. Suitable methods include, but are not limited to: a FRET assay (including fluorescence quenching assays); a BRET assay; an immunological assay; and an assay involving binding of a detectably labeled protein to an immobilized protein (e.g., binding of detectably labeled phosphorylated cyclin D1 to FBXW8, or binding of detectably labled FBXW8 to phosphorylated cyclin D1.

Immunological assays binding of a detectably labeled protein can be provided in a variety of formats. For example, immunoprecipitation assays can be designed, wherein the phosphorylated cyclin D1/FBXW8 polypeptide complex is detected by precipitating the complex with antibody specific for phosphorylated cyclin D1, FBXW8, or antibody specific for an immunodetectable tag of a phosphorylated cyclin D1 fusion protein and/or a FBXW8 fusion protein. In some formats, either phosphorylated cyclin D1 or FBXW8 can be immobilized directly or indirectly (e.g., by binding to an immoblizied antibody or other immobilized protein) on an insoluble support. Insoluble supports include, but are not limited to, plastic surfaces (e.g., polystyrene, and the like) such as a multi-well plate; beads, including magnetic beads, plastic beads, and the like; membranes (e.g., polyvinylpyrrolidone, nitrocellulose, and the like); etc. Bound complexes can be detected directly (e.g., by the presence of a detectable label of phosphorylated cyclin D1 or FBXW8 in a complex) or indirectly (e.g., by use of an antibody the specifically binds an immunodetectable tag present on one of the binding partners of the complex).

In cell-based embodiments, formation of complexes of FBXW8 and phosphorylated cyclin D1 can be detected in a variety of ways. For example, after contacting the cell with the agent and incubating for a sufficient amount of time, the presence or absence of complexes can be detected. This can be accomplished by producing cell extracts by, after allowing time for production of phosphorylated cyclin D1 and FBXW8 and for activity of FBXW8 in ubiquitination of phosphorylated cyclin D1, lysing the cells and examining lysates for the phosphorylated cyclin D1-FBXW8 complexes (e.g., by detection of a detectable label(s) on the binding partners in the complex or use of antibodies that specifically bind a binding partner in the complex). Alternatively or in addition, formation of phosphorylated cyclin D1-FBXW8 complexes can be detected in the cell cytoplasm (e.g., by detection of a detectable label(s) on the binding partners in the complex or use of antibodies that specifically bind a binding partner in the complex).

Cells used the assays can be genetically modified with expression vectors that provide for production of phosphorylated cyclin D1 and/or FBXW8 in a suitable eukaryotic cell, as described above, and may comprise genetically encodable detectable tags.

Identification of Agent that Modulate Ubiquitination of Phosphorylated Cyclin D1 Mediated by FBXW8

In one embodiment, the method involves combining (e.g., in a test sample in vitro or in a cell) a test agent, phosphorylated cyclin D1, FBXW8, and components necessary for FBXW8-mediated ubiquitination of phosphorylated cyclin D1 (e.g., ubiquitin and E1 and E2; or a ubiquitinated E2) under conditions suitable for ubiquitination of phosphorylated cyclin D1. Assays to assess the effect of a test agent upon FBXW8-mediated ubiquitination of phosphorylated cyclin D1 can be conducted in vitro (i.e., in a cell-free assay) or in vivo (i.e., in a cell).

Ubiquitination assays can involve assessing a change in moleculare weight of cyclin D1. Since ubiquitination of a substrate protein is associated with an increase in molecular weight, ubiquitinated cyclin D1 can be detected using any suitable method to assess a change in molecular weight of cyclin D1 relative to a molecular weight unubiquitinated cyclin D1. For example, anti-cyclin D1 antibodies can be used to detect cyclin D1 in assays that provide for separation by molecular weight (e.g., SDS-PAGE). Alternatively or in addition, such assays can use a cyclin D1 fusion protein having a detectable tag, and the detectable tag detected to facilitate assessment of ubiquitination of cyclin D1.

Alternatively, ubiquitination assays can use a tagged ubiquitin moiety (tag-Ub), which can be tagged as discussed above. Ubiquitination of phosphorylated cyclin D1 can be detected by assaying for the presence of cyclin D1 having the tagged ubiquitin. Exemplary assays for detecting agents that modulate ubiquitination of a a substrate protein are described in for example, Sjolander et al. (1991) Anal. chem. 63:2338-2345; Szabo et al. (1995) Curr. Opin. Struct. Biol. 5:699-705; and U.S. Pat. Ser. No. 6,329,171 to Kapeller-Libermann et al.; Zhu et al. (1997) Journal of Biological Chemistry 272:51-57, Mitch et al. (1999) American Journal of Physiology 276: C1132-C1138; Liu et al. (1999) Molecular and Cell Biology 19:3029-3038; Ciechanover et al. (1994) The FASEB Journal 8:182-192; Chiechanover (1994) Biol. Chem. Hoppe-Seyler 375:565-581; Hershko et al. (1998) Annual Review of Biochemistry 67:425-479; Swartz (1999) Annual Review of Medicine 50:57-74, Ciechanover (1998) EMBO Journal 17:7151-7160; and D'Andrea et al. (1998) Critical Reviews in Biochemistry; and Molecular Biology 33:337-352).

In one format, the assay is conducted in a cell-free system using a reaction mixture including isolated phosphorylated cyclin D1 (or cyclin D1, a source of phosphate (e.g, ATP), and MAPK included in the reaction mixture), FBXW8, ubiquitin, and other cellular components necessary to effect ubiquitination of phosphorylated cyclin D1 (e.g., by including an appropriate cell extract in the reaction mixture). FBXW8-containing E3 ligase complexes can be isolated from appropriate cells for use in such in vitro assays. The test agent is added to the reaction mixture, the reaction mixture incubated for a time sufficient to allow for ubiquitination of phosphorylated cyclin D1 in the absence of the test agent, and the effect of the agent upon cyclin D1 ubiquitination levels assessed.

In another format, the assay is conducted in a cell, which expresses endogenous components necessary for the ubiquitination assays and/or can be genetically modified to express one or more of cyclin D1 and FBXW8. The cell is contacted with the test agent and incubated for a time sufficient to allow for ubiquitination of phosphorylated cyclin D1 in the absence of the test agent, and the effect of the agent upon cyclin D1 ubiquitination levels assessed (e.g., by detecting a change in ubiquitinated cyclin D1 levels, which may be detected as a ratio of total cyclin D1 or phosphorylated cyclin D1).

Level of Cyclin D1 and/or Phosphorylated Cyclin D1 and/or Ubiquitinated Cyclin D1 In Cells

In some embodiments, a subject screening method involves determining the effect of a test agent on the level of total cyclin D1 (phosphorylated or unphosphorylated, ubiquitinated or non-ubiquitinated), phosphorylated cyclin D1, and/or ubiquitinated cyclin D1 in a cell in the presence of FBXW8 protein. In such embodiments, the method involves contacting a cell that produces FBXW8 (particularly a cell genetically modified to produce a recombinant FBXW8) and phosphorylated cyclin D1 with a test agent; and determining the effect, if any, of the test agent on the level of total cyclin D1, phosphorylated cyclin D1, and/or ubiquitinated cyclin D1 in the cell.

Whether a test agent modulates FBXW8-mediated ubiquitination of phosphorylated cyclin D1 and/or FBXW8-induced degradation of phosphorylated cyclin D1 can be determined by any known method for determining the level of a particular protein in a cell. In some embodiments, the assay is an immunological assay, using a cyclin-D I-specific antibody. Such methods include, but are not limited to, immunoprecipitating cyclin-D1 from a cellular extract, and analyzing the immunoprecipitated cyclin-D1 by sodium dodecyl sulfate polyacrylamide gel electrophoresis (SDS-PAGE); detecting a detectable fusion partner in a cell that produces a fusion protein that includes cyclin-D1 and a fusion partner that provides a detectable signal; standard SDS-PAGE and immunoblotting (e.g., transfer of proteins from a gel generated during SDS-PAGE to a membrane, and probing the membrane with detectably labeled antibodies) of cyclin-D1 from cells producing cyclin-D1.

In other embodiments, the assay is an assay that detects a tag present in a a cyclin-D1 fusion protein. The tag can provide for, e.g., an optically detectable signal or an immunodetectable signal. Such tags can be detected in extracts or, particularly where the tag is a fluorescent tag, the total cyclin D1 can be assessed in whole cells (e.g,. using fluorescent microscopy).

Total cyclin D1 can be readily determined by, e.g., immunoblotting nuclear and cytoplasmic fractions with cyclin D1-specific antibody, or by detecting a tag of a tagged cyclin D1 in such fractions. The ratio of cytoplasmic to nuclear cyclin D1 can also be determined in a similar fashion. Phosphorylated cyclin D1 can also be detected in cytoplasmic and, optionally, nuclear fractions using antibodies specific for phosphorylated cyclin D1. In order to ensure that the effect on total cyclin D1, phosphorylated cyclin D1, and/or ubiquitinated cyclin D1 is specific to FBXW8-mediated activitiy, the assay can be repeated (in series or parallel) in a negative control in which FBXW8 activity is inhibited (e.g., due to the presence of a dominant negative FBXW8 mutant or, in a cell-based assay, due to the presence of siRNA specific for FBXW8 or in a FBXW8-knockout cell) or in a control in which, for example, FBXW8 is overexpressed to effectively dilute the effect of the test agent. Other means to determining that the agent specifically affects FBXW8-mediated ubiquitination will be readily apparent to the ordinarily skilled artisan, so as to confirm that the effect observed in the presence of the test agent is specific for interaction of FBXW8 with phosphorylated cyclin D1 (e.g., the agent does not detectably affect MAPK activity in phosphorylation of cyclin D1, i.e., the agent is not a modulator of MAPK activity, such as an MAPK inhibitor).

Identification of Agents that Modulate MAPK Activity in Cyclin D1 Phosphorylation

In general, assays to identify agents that modulate phosphorylation of cyclin D1 by MAPK involve contacting a test agent with unphosphorylated cyclin D1 and MAPK, wherein the cyclin D1 and MAPK may be present in a cell-free assay or within a cell. Where cells are used in the assay, the cell may be a cell recombinant for one or both of cyclin D1 and mk. In either in vitro or cell-based assays, one or both of cyclin D1 and MAPK may be detectably labeled. If both are detectably labeled, then the labels are different so as to provide for signals that are distinguishable. The agent is contacted with the cyclin D1 and MAPK for a time sufficient for the interaction between phosphorylated cyclin D1 and mk to occur, and the effect of the agent detected. Effects on interaction of cyclin D1 and MAPK can be detected by detecting an effect on binding of MAPK and cyclin D1, or an effect on activity of MAPK in mediating phosphorylation of cyclin D1. Exemplary assay formats are provided below.

An agent that modulates (increases or decreases) MAPK-cyclin D1 interactions (as detected directly (e.g., by detecting binding of MAPK and cyclin D1) or indirectly (e.g., by detecting phosphorylation of cyclin D1) is an agent that provides for a change of at least about 10%, at least about 20%, at least about 30%, at least about 50%, at least about 75%, at least about 100%, at least about 2.5-fold, at least about 3-fold, at least about 4-fold, at least about 5-fold, at least about 10-fold, at least about 20-fold, or at least about 50-fold, in the detected parameter associated with MAPK-cyclin D1 interaction (e.g., MAPK-cyclin D1 binding; phosphorylated cyclin D1, and the like).

Assays Assessing MAPK Binding with Cyclin D1

The screening methods provided herein include assays to identify an agent that modulates binding of MAPK with cyclin D1. Such assays can be conducted in vitro (e.g., in vitro binding assays) or in vivo (e.g, using cells having detectably labeled MAPK, detectably labeled cyclin D1, or both). Exemplary assays are described below.

The assay can involve, for example, contacting cyclin D1 and MAPK with a test agent, and directly determining the effect, if any, of the test agent on the binding of cyclin D1 and MAPK. This methods can be conducted in vitro (i.e., cell-free) in a reaction mixture, using isolated polypeptides. Where desired or required, the in vitro assay reaction mixture can comprise cell extracts (e.g., cell cytoplasm extracts) so as to provide cellular components required for interaction between MAPK and cyclin D1. The cell extract is prepared from a cell in which MAPK-mediated phosphorylation of cyclin D1 occurs (e.g., due to endogenous activity or activity as a result of genetic modification). Alternatively, the assay can be performed in a cell-based assay, where the cell can provide for assay components by expression from an endogenous or non-endogenous (recombinant) nucleic acid.

Formation of a binding complex between cyclin D1 and MAPK can be detected using any known method. Suitable methods include, but are not limited to: a FRET assay (including fluorescence quenching assays); a BRET assay; an immunological assay; and an assay involving binding of a detectably labeled protein to an immobilized protein (e.g., binding of detectably labeled cyclin D1 to MAPK, or binding of detectably labled MAPK to cyclin D1.

Immunological assays binding of a detectably labeled protein can be provided in a variety of formats. For example, immunoprecipitation assays can be designed, wherein the cyclin D1/MAPK complex is detected by precipitating the complex with antibody specific for cyclin D1, MAPK, or antibody specific for an immunodetectable tag of a cyclin D1 fusion protein and/or a MAPK fusion protein. In some formats, either cyclin D1 or MAPK can be immobilized directly or indirectly (e.g., by binding to an immoblized antibody or other immobilized protein) on an insoluble support. Insoluble supports include, but are not limited to, plastic surfaces (e.g., polystyrene, and the like) such as a multi-well plate; beads, including magnetic beads, plastic beads, and the like; membranes (e.g., polyvinylpyrrolidone, nitrocellulose, and the like); etc. Bound complexes can be detected directly (e.g., by the presence of a detectable label of cyclin D1 or MAPK in a complex) or indirectly (e.g., by use of an antibody the specifically binds an immunodetectable tag present on one of the binding partners of the complex).

In cell-based embodiments, formation of complexes of MAPK and cyclin D1 can be detected in a variety of ways. For example, after contacting the cell with the agent and incubating for a sufficient amount of time, the presence or absence of complexes can be detected. This can be accomplished by producing cell extracts by, after allowing time for production of cyclin D1 and MAPK and for activity of MAPK in phosphorylation of cyclin D1, lysing the cells and examining lysates for the cyclin D1-FBXW8 complexes (e.g., by detection of a detectable label(s) on the binding partners in the complex or use of antibodies that specifically bind a binding partner in the complex). Alternatively or in addition, formation of cyclin D1-MAPK complexes can be detected in the cell cytoplasm (e.g., by detection of a detectable label(s) on the binding partners in the complex or use of antibodies that specifically bind a binding partner in the complex).

Cells used the assays can be genetically modified with expression vectors that provide for production of cyclin D1 and/or MAPK in a suitable eukaryotic cell, as described above, and may comprise genetically encodable detectable tags.

Assays Assessing Phosphorylated Cyclin D1 (Cell-free or Cell-based)

In some embodiments, the screening method involves determining the effect of a test agent on the level of phosphorylated cyclin D1 produced by MAPK either in vitro or in vivo.

In vitro assays generally involve isolated cyclin D1, isolated MAPK, and a source of phosphate (e.g., ATP). Cell extracts of cells that have endogenous MAPK-mediated cyclin D1 phosphorylation activity, or are genetically modified to have such activity, can be used in the assays to provide other cellular components as may be necessary.

Cell-based methods generally involve contacting a cell that produces MAPK (particularly a cell genetically modified to produce a recombinant MAPK) and cyclin D1 (endogenous or recombinant cyclin D1) with a test agent; and determining the effect, if any, of the test agent on the level of phosphorylated cyclin D1.

Whether a test agent modulates MAPK-mediated phosphorylation of cyclin D1 can be determined by any known method for determining the level of a particular protein in a cell. In some embodiments, the assay is an immunological assay, using an antibody specific for phosphorylated cyclin-D1. Such methods include, but are not limited to, immunoprecipitating phosphorylated cyclin-D1 from a cellular extract, and analyzing the immunoprecipitated cyclin-D1 (e.g., by sodium dodecyl sulfate polyacrylamide gel electrophoresis (SDS-PAGE); detecting a detectable tag of a cyclin D1 fusion protein in a cell genetically modified to produce the cyclin D1 fusion protein and assaying the cyclin D1 fusion protein for the presence of a phosphorylated Thr286 residue; analyzing cell lysates by Western blot (or other like technique) using anti-phosphorylated cyclin D1 antibodies.

In other embodiments, the in vitro or cell-based assay includes a radiodetectably source of phosphate (e.g., ³²P), and the level of phosphorylated cyclin D1 is assessed by detection of the incorporation of the radiolabel into the phosphorylated cyclin D1 polypeptide.

In order to ensure that the effect on phosphorylated cyclin D1 is specific to MAPK-mediated activity, the assay can be repeated (in series or parallel) using negative controls (e.g., controls for comparison in which MAPK activity is inhibited (e.g., due to the presence of a specific MAPK inhibitor or, in cell-based assays, due to the presence of siRNA specific for MAPK or use of a MAPK-knockout cell)) or MAPK can be overexpressed in a cell contacted with the test agent to show that restoration of MAPK activity diminishes the effect of the agent. Other means to determinig that the agent specifically affects MAPK-phosphorylation of cyclin D1 will be readily apparent to the ordinarily skilled artisan, so as to confirm that the effect observed in the presence of the test agent is specific for interaction of FBXW8 with phosphorylated cyclin D1 (e.g., the agent does not detectably affect FBXW8-mediated cyclin D1 ubiquitination, i.e., the agent is not a modulator of FBXW8 activity, such as an FBXW8 ubiquitination activity inhibitor).

Agents that Modulate Cyclin D1 Phosphorylation and/or Ubiquitin-mediated Degradation

Agents that modulate cellular proliferation through modulating cyclin D1 phosphorylation and/or ubiquitin-mediated degradation can be providing in pharmaceutical formulations and administered to a subject for treatment of an appropriate condition. For example, where the agent provides for a decrease in cyclin D1 degradation (e.g., by inhibiting cyclin D1 phosphorylation and/or inhibiting cyclin D1 ubiquitination), the agent has activity in inhibiting cellular proliferation. Such agents are of interest for use in treatment of cellular proliferative diseases, such as cancer. Where the agent provides for an increase in cyclin D1 degradation (e.g., by promoting cyclin D1 phosphorylation and/or promoting cyclin D1 ubiquitination), the agent has activity in increasing cellular proliferation.

The inventors have identified siRNAs as exemplary agents that provide for inhibition of cellular proliferation. These exemplary agents are described in more detail below, as are methods of formulation and delivery of agents of interest.

siNAs as Agents For Expression-based Inhibition of FBXW8, CUL1, and/or CUL7

In one embodiment, inhibition of cellular proliferation is accomplished through RNA interference (RNAi) by contacting a cell with a small nucleic acid molecule, such as a short interfering nucleic acid (siNA), a short interfering RNA (siRNA), a double-stranded RNA (dsRNA), a micro-RNA (miRNA), or a short hairpin RNA (shRNA) molecule, or modulation of expression of a small interfering RNA (siRNA) so as to provide for decreased levels of at least one of FBXW8, CUL1, or CUL7 (e.g., through a decrease in mRNA levels and/or a decrease in polypeptide levels).

The term “short interfering nucleic acid”, “siNA”, “short interfering RNA”, “siRNA”, “short interfering nucleic acid molecule”, “short interfering oligonucleotide molecule”, or “chemically-modified short interfering nucleic acid molecule” as used herein refers to any nucleic acid molecule capable of inhibiting or down regulating gene expression, for example by mediating RNA interference “RNAi” or gene silencing in a sequence-specific manner. Design of RNAi molecules when given a target gene are routine in the art. See also US 2005/0282188 (which is incorporated herein by reference) as well as references cited therein. See, e.g., Pushparaj et al. Clin Exp Pharmacol Physiol. 2006 May-June;33(5-6):504-10; Lutzelberger et al. Handb Exp Pharmacol. 2006;(173):243-59; Aronin et al. Gene Ther. 2006 March;13(6):509-16; Xie et al. Drug Discov Today. 2006 January;11(1-2):67-73; et al. Curr Med Chem. 2005;12(26):3143-61; and Pekaraik et al. Brain Res Bull. 2005 Dec. 15;68(1-2):115-20. Epub 2005 Sep. 9.

Methods for design and production of siRNAs to a desired target are known in the art, and their application to FBXW8, CUL1 and CUL7 genes for the purposes disclosed herein will be readily apparent to the ordinarily skilled artisan, as are methods of production of siRNAs having modifications (e.g., chemical modifications) to provide for, e.g., enhanced stability, bioavailability, and other properties to enhance use as therapeutics. In addition, methods for formulation and delivery of siRNAs to a subject are also well known in the art. See, e.g., US 2005/0282188; US 2005/0239731; US 2005/0234232; US 2005/0176018; US 2005/0059817; US 2005/0020525; US 2004/0192626; US 2003/0073640; US 2002/0150936; US 2002/0142980; and US2002/0120129, each of which are incorporated herein by reference.

Publicly available tools to facilitate design of siRNAs are available in the art. See, e.g., DEQOR: Design and Quality Control of RNAi (available on the internet at cluster-1.mpi-cbg.de/Deqor/deqor.html). See also, Henschel et al. Nucleic Acids Res. 2004 Jul. 1;32(Web Server issue):W113-20. DEQOR is a web-based program which uses a scoring system based on state-of-the-art parameters for siRNA design to evaluate the inhibitory potency of siRNAs. DEQOR, therefore, can help to predict (i) regions in a gene that show high silencing capacity based on the base pair composition and (ii) siRNAs with high silencing potential for chemical synthesis. In addition, each siRNA arising from the input query is evaluated for possible cross-silencing activities by performing BLAST searches against the transcriptome or genome of a selected organism. DEQOR can therefore predict the probability that an mRNA fragment will cross-react with other genes in the cell and helps researchers to design experiments to test the specificity of siRNAs or chemically designed siRNAs.

Non limiting examples of target sites for design of siNA molecules for each of FBXW8, CUL1, and CUL7 are provided in the Examples below. Specifically, the following FBXW8, CUL1, and CUL7 siRNA oligonucleotides target sites were selected to knockdown endogenous expression: FBXW8 (AAGAUGUGCACAGGUGAGCAA), CUL1 (AAUAGACAUUGGGUUCGCCGU), and CUL7 (AAGGAUGAGAUCUAUGCCAAC). Additional target sites can be readily identified using the tools available to the ordinarily skilled artisan as discussed above.

It should be understood that the sequences provided above are the target sequences of the mRNAs encoding the target gene, and that the siRNA oligonucleotides used would comprise a sequence complementary to the target.

siNA molecules can be of any of a variety of forms. For example the siNA can be a double-stranded polynucleotide molecule comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof. siNA can also be assembled from two separate oligonucleotides, where one strand is the sense strand and the other is the antisense strand, wherein the antisense and sense strands are self-complementary. In this embodiment, each strand generally comprises nucleotide sequence that is complementary to nucleotide sequence in the other strand; such as where the antisense strand and sense strand form a duplex or double stranded structure, for example wherein the double stranded region is about 15 to about 30, e.g., about 15, 16, 17, 18, 19, 20, 21, 22, 23, 24, 25, 26, 27, 28, 29 or 30 base pairs; the antisense strand comprises nucleotide sequence that is complementary to nucleotide sequence in a target nucleic acid molecule or a portion thereof and the sense strand comprises nucleotide sequence corresponding to the target nucleic acid sequence or a portion thereof (e.g., about 15 to about 25 or more nucleotides of the siNA molecule are complementary to the target nucleic acid or a portion thereof).

Alternatively, the siNA can be assembled from a single oligonucleotide, where the self-complementary sense and antisense regions of the siNA are linked by a nucleic acid-based or non-nucleic acid-based linker(s). The siNA can be a polynucleotide with a duplex, asymmetric duplex, hairpin or asymmetric hairpin secondary structure, having self-complementary sense and antisense regions, wherein the antisense region comprises nucleotide sequence that is complementary to nucleotide sequence in a separate target nucleic acid molecule or a portion thereof and the sense region having nucleotide sequence corresponding to the target nucleic acid sequence or a portion thereof.

The siNA can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide can be processed either in vivo or in vitro to generate an active siNA molecule capable of mediating RNAi. The siNA can also comprise a single stranded polynucleotide having nucleotide sequence complementary to nucleotide sequence in a target nucleic acid molecule or a portion thereof (e.g., where such siNA molecule does not require the presence within the siNA molecule of nucleotide sequence corresponding to the target nucleic acid sequence or a portion thereof), wherein the single stranded polynucleotide can further comprise a terminal phosphate group, such as a 5′-phosphate (see for example Martinez et al., 2002, Cell., 110, 563-574 and Schwarz et al., 2002, Molecular Cell, 10, 537-568), or 5′,3′-diphosphate.

In certain embodiments, the siNA molecule contains separate sense and antisense sequences or regions, wherein the sense and antisense regions are covalently linked by nucleotide or non-nucleotide linkers molecules as is known in the art, or are alternately non-covalently linked by ionic interactions, hydrogen bonding, van der Waals interactions, hydrophobic interactions, and/or stacking interactions. In certain embodiments, the siNA molecules comprise nucleotide sequence that is complementary to nucleotide sequence of a target gene. In another embodiment, the siNA molecule interacts with nucleotide sequence of a target gene in a manner that causes inhibition of expression of the target gene.

As used herein, siNA molecules need not be limited to those molecules containing only RNA, but further encompasses chemically-modified nucleotides and non-nucleotides. In certain embodiments, the short interfering nucleic acid molecules of the invention lack 2′-hydroxy (2′-OH) containing nucleotides. siNAs do not necessarily require the presence of nucleotides having a 2′-hydroxy group for mediating RNAi and as such, siNA molecules of the invention optionally do not include any ribonucleotides (e.g., nucleotides having a 2′-OH group). Such siNA molecules that do not require the presence of ribonucleotides within the siNA molecule to support RNAi can however have an attached linker or linkers or other attached or associated groups, moieties, or chains containing one or more nucleotides with 2′-OH groups. Optionally, siNA molecules can comprise ribonucleotides at about 5, 10, 20, 30, 40, or 50% of the nucleotide positions. The modified short interfering nucleic acid molecules of the invention can also be referred to as short interfering modified oligonucleotides “siMON.”

As used herein, the term siNA is meant to be equivalent to other terms used to describe nucleic acid molecules that are capable of mediating sequence specific RNAi, for example short interfering RNA (siRNA), double-stranded RNA (dsRNA), micro-RNA (miRNA), short hairpin RNA (shRNA), short interfering oligonucleotide, short interfering nucleic acid, short interfering modified oligonucleotide, chemically-modified siRNA, post-transcriptional gene silencing RNA (ptgsRNA), and others. In addition, as used herein, the term RNAi is meant to be equivalent to other terms used to describe sequence specific RNA interference, such as post transcriptional gene silencing, translational inhibition, or epigenetics. For example, siNA molecules of the invention can be used to epigenetically silence a target gene at both the post-transcriptional level or the pre-transcriptional level. In a non-limiting example, epigenetic regulation of gene expression by siNA molecules of the invention can result from siNA mediated modification of chromatin structure or methylation pattern to alter gene expression (see, for example, Verdel et al., 2004, Science, 303, 672-676; Pal-Bhadra et al., 2004, Science, 303, 669-672; Allshire, 2002, Science, 297, 1818-1819; Volpe et al., 2002, Science, 297, 1833-1837; Jenuwein, 2002, Science, 297, 2215-2218; and Hall et al., 2002, Science, 297, 2232-2237).

siNA molecules contemplated herein can comprise a duplex forming oligonucleotide (DFO) see, e.g., WO 05/019453; and US 2005/0233329, which are incorporated herein by reference). siNA molecules also contemplated herein include multifunctional siNA, (see, e.g., WO 05/019453 and US 2004/0249178). The multifunctional siNA can comprise sequence targeting, for example, two regions of FBXW8, CUL1, and/or CUL7.

siNA molecules contemplated herein can comprise an asymmetric hairpin or asymmetric duplex. By “asymmetric hairpin” as used herein is meant a linear siNA molecule comprising an antisense region, a loop portion that can comprise nucleotides or non-nucleotides, and a sense region that comprises fewer nucleotides than the antisense region to the extent that the sense region has enough complementary nucleotides to base pair with the antisense region and form a duplex with loop. For example, an asymmetric hairpin siNA molecule can comprise an antisense region having length sufficient to mediate RNAi in a cell or in vitro system (e.g. about 15 to about 30, or about 15, 16, 17, 18, 19, 20, 21, 22, 23, 24, 25, 26, 27, 28, 29, or 30 nucleotides) and a loop region comprising about 4 to about 12 (e.g., about 4, 5, 6, 7, 8, 9, 10, 11, or 12) nucleotides, and a sense region having about 3 to about 25 (e.g., about 3, 4, 5, 6, 7, 8, 9, 10, 11, 12, 13, 14, 15, 16, 17, 18, 19, 20, 21, 22, 23, 24, or 25) nucleotides that are complementary to the antisense region. The asymmetric hairpin siNA molecule can also comprise a 5′-terminal phosphate group that can be chemically modified. The loop portion of the asymmetric hairpin siNA molecule can comprise nucleotides, non-nucleotides, linker molecules, or conjugate molecules as described herein.

By “asymmetric duplex” as used herein is meant a siNA molecule having two separate strands comprising a sense region and an antisense region, wherein the sense region comprises fewer nucleotides than the antisense region to the extent that the sense region has enough complementary nucleotides to base pair with the antisense region and form a duplex. For example, an asymmetric duplex siNA molecule of the invention can comprise an antisense region having length sufficient to mediate RNAi in a cell or in vitro system (e.g. about 15 to about 30, or about 15, 16, 17, 18, 19,20, 21, 22, 23, 24, 25, 26, 27, 28, 29, or 30 nucleotides) and a sense region having about 3 to about 25 (e.g., about 3, 4, 5, 6, 7, 8, 9, 10, 11, 12, 13, 14, 15, 16, 17, 18, 19, 20, 21, 22, 23, 24, or 25) nucleotides that are complementary to the antisense region.

Stability and/or half-life of siRNAs can be improved through chemically synthesizing nucleic acid molecules with modifications (base, sugar and/or phosphate) can prevent their degradation by serum ribonucleases, which can increase their potency (see e.g., Eckstein et al., International Publication No. WO 92/07065; Perrault et al., 1990 Nature 344, 565; Pieken et al., 1991, Science 253, 314; Usman and Cedergren, 1992, Trends in Biochem. Sci. 17, 334; Usman et al., International Publication No. WO 93/15187; and Rossi et al., International Publication No. WO 91/03162; Sproat, U.S. Pat. No. 5,334,711; Gold et al., U.S. Pat. No. 6,300,074; and Burgin et al., supra; all of which are incorporated by reference herein, describing various chemical modifications that can be made to the base, phosphate and/or sugar moieties of the nucleic acid molecules described herein. Modifications that enhance their efficacy in cells, and removal of bases from nucleic acid molecules to shorten oligonucleotide synthesis times and reduce chemical requirements are desired.

For example, oligonucleotides are modified to enhance stability and/or enhance biological activity by modification with nuclease resistant groups, for example, 2′-amino, 2′-C-allyl, 2′-fluoro, 2′-O-methyl, 2′-O-allyl, 2′-H, nucleotide base modifications (for a review see Usman and Cedergren, 1992, TIBS. 17, 34; Usman et al., 1994, Nucleic Acids Symp. Ser. 31, 163; Burgin et al., 1996, Biochemistry, 35, 14090). Sugar modification of nucleic acid molecules have been extensively described in the art (see Eckstein et al., International Publication PCT No. WO 92/07065; Perrault et al. Nature, 1990, 344, 565-568; Pieken et al. Science, 1991, 253, 314-317; Usman and Cedergren, Trends in Biochem. Sci., 1992, 17, 334-339; Usman et al. International Publication PCT No. WO 93/15187; Sproat, U.S. Pat. No. 5,334,711 and Beigelman et al., 1995, J. Biol. Chem., 270, 25702; Beigelman et al., International PCT publication No. WO 97/26270; Beigelman et al., U.S. Pat. No. 5,716,824; Usman et al., U.S. Pat. No. 5,627,053; Woolf et al., International PCT Publication No. WO 98/13526; Thompson et al., U.S. Ser. No. 60/082,404 which was filed on Apr. 20, 1998; Karpeisky et al., 1998, Tetrahedron Lett., 39, 1131; Eamshaw and Gait, 1998, Biopolymers (Nucleic Acid Sciences), 48, 39-55; Verma and Eckstein, 1998, Annu. Rev. Biochem., 67, 99-134; and Burlina et al., 1997, Bioorg. Med. Chem., 5, 1999-2010; each of which are hereby incorporated in their totality by reference herein). In view of such teachings, similar modifications can be used as described herein to modify the siNA nucleic acid molecules of disclosed herein so long as the ability of siNA to promote RNAi is cells is not significantly inhibited.

Short interfering nucleic acid (siNA) molecules having chemical modifications that maintain or enhance activity are contemplated herein. Such a nucleic acid is also generally more resistant to nucleases than an unmodified nucleic acid. Accordingly, the in vitro and/or in vivo activity should not be significantly lowered. Nucleic acid molecules delivered exogenously are generally selected to be be stable within cells at least for a period sufficient for transcription and/or translation of the target RNA to occur and to provide for modulation of production of the encoded mRNA and/or polypeptide so as to facilitate reduction of the level of the target gene product.

Production of RNA and DNA molecules can be accomplished synthetically and can provide for introduction of nucleotide modifications to provide for enhanced nuclease stability. (see, e.g., Wincott et al., 1995, Nucleic Acids Res. 23, 2677; Caruthers et al., 1992, Methods in Enzymology 211, 3-19, incorporated by reference herein. In one embodiment, nucleic acid molecules of the invention include one or more (e.g., about 1, 2, 3, 4, 5, 6, 7, 8, 9, 10, or more) G-clamp nucleotides, which are modified cytosine analogs which confer the ability to hydrogen bond both Watson-Crick and Hoogsteen faces of a complementary guanine within a duplex, and can provide for enahcned affinity and specificity to nucleic acid targets (see, e.g., Lin et al. 1998, J. Am. Chem. Soc., 120, 8531-8532). In another example, nucleic acid molecules can include one or more (e.g., about 1, 2, 3, 4, 5, 6, 7, 8, 9, 10, or more) LNA “locked nucleic acid” nucleotides such as a 2′,4′-C methylene bicyclo nucleotide (see, e.g., Wengel et al., WO 00/66604 and WO 99/14226).

siNA molecules can be provided as conjugates and/or complexes, e.g., to facilitate delivery of siNA molecules into a cell. Exemplary conjugates and/or complexes includes those composed of an siNA and a small molecule, lipid, cholesterol, phospholipid, nucleoside, antibody, toxin, negatively charged polymer (e.g., protein, peptide, hormone, carbohydrate, polyethylene glycol, or polyamine). In general, the transporters described are designed to be used either individually or as part of a multi-component system, with or without degradable linkers. These compounds can improve delivery and/or localization of nucleic acid molecules into cells in the presence or absence of serum (see, e.g., U.S. Pat. No. 5,854,038). Conjugates of the molecules described herein can be attached to biologically active molecules via linkers that are biodegradable, such as biodegradable nucleic acid linker molecules.

Administration and Formulation of Agents

Formulation of an agent of interest for delivery to a subject, as well as method of delivery of agents (including siNA molecules as described above), are available in the art. These include formulations and delivery methods to effect systemic delivery of an agent, as well as formulation and delivery methods to effect local delivery of an agent (e.g., to effect to a particular organ or compartment (e.g., to effect delivery to a tumor located in breast tissue, colon tissue, liver tissue, central nervous system (CNS), etc.)). Agents (such as an siNA) can be formulated to include a delivery vehicle for administration to a subject, carriers and diluents and their salts, and/or can be present in pharmaceutically acceptable formulations.

Suitable formulations at least in part depend upon the use or the route of entry, for example parenteral, oral, or transdermal. The term “parenteral” as used herein includes percutaneous, subcutaneous, intravascular (e.g., intravenous), intramuscular, or intrathecal injection or infusion techniques and the like.Formulations include pharmaceutically acceptable salts of an agent of interest, e.g., acid addition salts.

In one embodiment, compounds (such as siNA molecules) are administered to a subject by systemic administration in a pharmaceutically acceptable composition or formulation. By “systemic administration” is meant in vivo systemic absorption or accumulation of drugs in the blood stream to facilitate distribution through the body. Systemic administration routes include, e.g., intravenous, subcutaneous, portal vein, intraperitoneal, inhalation, oral, intrapulmonary and intramuscular.

Formulations of agents can also be administered orally, topically, parenterally, by inhalation or spray, or rectally in dosage unit formulations containing pharmaceutically acceptable carriers, adjuvants and/or vehicles. Pharmaceutically acceptable carriers or diluents for therapeutic use are well known in the pharmaceutical art, and are described, for example, in Remington's Pharmaceutical Sciences, Mack Publishing Co. (A. R. Gennaro edit. 1985), hereby incorporated herein by reference. For example, preservatives, stabilizers, dyes and flavoring agents can be provided. These include sodium benzoate, sorbic acid and esters of p-hydroxybenzoic acid. In addition, antioxidants and suspending agents can be used.

A pharmaceutically effective dose is that dose required to prevent, inhibit the occurrence, or treat (alleviate a symptom at least to some extent) of a disease state. The pharmaceutically effective dose depends on the type of disease, the composition used, the route of administration, the type of subject being treated, subject-dependent characteristics under consideration, concurrent medication, and other factors that those skilled in the medical arts will recognize. Generally, an amount between 0.1 mg/kg and 100 mg/kg body weight/day of active ingredients is administered.

Formulations and methods of delivery of agents to a tumor are well known in the art. Local delivery to tumor can be accomplished by, for example, intra or peritumoral injection, especially where a tumor is a solid tumor or semi-solid tumor (e.g., Hodgkins lymphoma, non-Hodgkins lymphoma, and the like). Local injection into a tissue defining a biological compartment (e.g., ovary, intrathecal space, synovial space, and the like) is also of interest.

Formulations and methods of delivery of agents (including nucleic acid molecules) to the liver are known in the art, see, e.g., Wen et al., 2004, World J Gastroenterol., 10, 244-9; Murao et al., 2002, Pharm Res., 19, 1808-14; Liu et al., 2003, Gene Ther., 10, 180-7; Hong et al., 2003, J Pharm Pharmacol., 54, 51-8; Herrmann et al., 2004, Arch Virol., 149, 1611-7; and Matsuno et al., 2003, Gene Ther., 10, 1559-66.

Where pulmonary delivery is desired, agents (e.g., nucleic acid molecules) can be administered by, e.g., inhalation of an aerosol or spray dried formulation administered by an inhalation device (e.g., nebulizer, insufflator, metered dose inhaler, and the like), providing uptake of the agent into pulmonary tissues. Solid particulate compositions containing respirable dry particles of micronized compositions containing a compound of interest (e.g., nucleic acid) can be prepared by standard techniques. A solid particulate composition can optionally contain a dispersant which serves to facilitate the formation of an aerosol. A suitable dispersant is lactose, which can be blended with the agent in any suitable ratio, such as a 1 to 1 ratio by weight. The active ingredient typically in about 0.1 to 100 w/w of the formulation. The agent can be delivered as a a suspension or solution formulation, and may involve use of a liquified propellant, e.g., a chlorofluorocarbon compound such as dichlorodifluoromethane, trichlorofluoromethane, dichlorotetrafluoroethane and mixtures thereof. Aerosol formulation can additionally contain one or more co-solvents, for example, ethanol, emulsifiers and other formulation surfactants, such as oleic acid or sorbitan trioleate, anti-oxidants and suitable flavoring agents. Other methods for pulmonary delivery are described in, for example US 2004/0037780, and U.S. Pat. No. 6,592,904; U.S. Pat. No. 6,582,728; U.S. Pat. No. 6,565,885, each of which are incorporated herein by reference.

Formulations and methods of delivery of agents (including nucleic acid molecules) to hematopoietic cells, including monocytes and lymphocytes, are known in the art, see, e.g., Hartmann et al., 1998, J. Phamacol. Exp. Ther., 285(2), 920-928; Kronenwett et al., 1998, Blood, 91(3), 852-862; Filion and Phillips, 1997, Biochim. Biophys. Acta., 1329(2), 345-356; Ma and Wei, 1996, Leuk. Res., 20(11/12), 925-930; and Bongartz et al., 1994, Nucleic Acids Research, 22(22), 4681-8. Such methods, as described above, include the use of free compound (e.g., oligonucleotide), cationic lipid formulations, liposome formulations including pH sensitive liposomes and immunoliposomes, and bioconjugates including oligonucleotides conjugated to fusogenic peptides, for delivery of compounds into hematopoietic cells.

Formulations and methods of delivery of agents (including nucleic acid molecules) to the skin or mucosa are known in the art. Such delivery systems include, e.g., aqueous and nonaqueous gels, creams, multiple emulsions, microemulsions, liposomes, ointments, aqueous and nonaqueous solutions, lotions, patches, suppositories, and tablets, and can contain excipients such as solubilizers, permeation enhancers (e.g., fatty acids, fatty acid esters, fatty alcohols and amino acids), and hydrophilic polymers (e.g., polycarbophil and polyvinylpyrolidone).

Delivery to the central nervous system (CNS) and/or peripheral nervous system can be accomplished by, for example, local administration of nucleic acids to nerve cells. Conventional approaches to CNS delivery that can be used include, but are not limited to, intrathecal and intracerebroventricular administration, implantation of catheters and pumps, direct injection or perfusion at the site of injury or lesion, injection into the brain arterial system, or by chemical or osmotic opening of the blood-brain barrier. Other approaches can include the use of various transport and carrier systems, for example though the use of conjugates and biodegradable polymers. See also, U.S. Pat. No. 6,180,613; WO 04/013280, describing delivery of nucleic acid molecules to the CNS, which are incorporated herein by reference.

Oral administration can be accomplished using pharmaceutical compositions containing an agent of interest (e.g., an siNA) formulated as tablets, lozenges, aqueous or oily suspensions, dispersible powders or granules, emulsion, hard or soft capsules, or syrups or elixirs. Such oral compositions can contain one or more such sweetening agents, flavoring agents, coloring agents or preservative agents in order to provide pharmaceutically elegant and palatable preparations. Tablets, which can be coated or uncoated, can be formulated to contain the active ingredient in admixture with non-toxic pharmaceutically acceptable excipients, e.g., inert diluents; such as calcium carbonate, sodium carbonate, lactose, calcium phosphate or sodium phosphate; granulating and disintegrating agents, for example, corn starch, or alginic acid; binding agents, for example starch, gelatin or acacia; and lubricating agents, for example magnesium stearate, stearic acid or talc. Where a coating is used, the coating delay disintegration and absorption in the gastrointestinal tract and thereby provide a sustained action over a longer period.

Where the formulation is an aqueous suspension, such can contain the active agent in a mixture with a suitable excipient(s). Such excipients can be, as appropriate, suspending agents (e.g., sodium carboxymethylcellulose, methylcellulose, hydropropyl-methylcellulose, sodium alginate, polyvinylpyrrolidone, gum tragacanth and gum acacia); dispersing or wetting agents; preservatives; coloring agents; and/or flavoring agents.

Suppositories, e.g., for rectal administration of agents, can be prepared by mixing the agent with a suitable non-irritating excipient that is solid at ordinary temperatures but liquid at the rectal temperature and will therefore melt in the rectum to release the drug. Such materials include cocoa butter and polyethylene glycols.

Dosage levels can be readily determined by the ordinarily skilled clinician, and can be modified as required, e.g., as required to modify a subject's response to therapy. In general dosage levels are on the order of from about 0.1 mg to about 140 mg per kilogram of body weight per day. The amount of active ingredient that can be combined with the carrier materials to produce a single dosage form varies depending upon the host treated and the particular mode of administration. Dosage unit forms generally contain between from about 1 mg to about 500 mg of an active ingredient.

The agents (including siNAs) can be administered to a subject in combination with other therapeutic compounds, e.g., so as to increase the overall therapeutic effect. For example, in the context of cancer therapy, it may be beneficial to administer the agent with another chemotherapy regimen (e.g., antibody-based therapy) and/or with agents that diminish undesirable side-effects. Examples of chemotherapeutic agents for use in combination therapy include, but are not limited to, daunorubicin, daunomycin, dactinomycin, doxorubicin, epirubicin, idarubicin, esorubicin, bleomycin, mafosfamide, ifosfamide, cytosine arabinoside, bis-chloroethyinitrosurea, busulfan, mitomycin C, actinomycin D, mithramycin, prednisone, hydroxyprogesterone, testosterone, tamoxifen, dacarbazine, procarbazine, hexamethylmelamine, pentamethylmelamine, mitoxantrone, amsacrine, chlorambucil, methylcyclohexylnitrosurea, nitrogen mustards, melphalan, cyclophosphamide, 6-mercaptopurine, 6-thioguanine, cytarabine, 5-azacytidine, hydroxyurea, deoxycoformycin, 4-hydroxyperoxycyclophosphor-amide, 5-fluorouracil (5-FU), 5-fluorodeoxyuridine (5-FUdR), methotrexate (MTX), colchicine, taxol, vincristine, vinblastine, etoposide (VP-16), trimetrexate, irinotecan, topotecan, gemcitabine, teniposide, cisplatin and diethylstilbestrol (DES).

Of particular interest are agents that a siNAs, as described above. Exemplary formulations and methods for the delivery of nucleic acid molecules are known in the art. For example, nucleic acid molecules can be administered to cells by a variety of methods known to those of skill in the art, including, but not restricted to, encapsulation in liposomes, by iontophoresis, or by incorporation into other vehicles, such as biodegradable polymers, hydrogels, cyclodextrins (see for example Gonzalez et al., 1999, Bioconjugate Chem., 10, 1068-1074; Wang et al., International PCT publication Nos. WO 03/47518 and WO 03/46185), poly(lactic-co-glycolic)acid (PLGA) and PLCA microspheres (see for example U.S. Pat. No. 6,447,796 and US Patent Application Publication No. U.S. 2002130430), biodegradable nanocapsules, and bioadhesive microspheres, or by proteinaceous vectors (O'Hare and Normand, International PCT Publication No. WO 00/53722). In another embodiment, the nucleic acid molecules of the invention can also be formulated or complexed with polyethyleneimine and derivatives thereof, such as polyethyleneimine-polyethyleneglycol-N-acetylgalactosamine (PEI-PEG-GAL) or polyethyleneimine-polyethyleneglycol-tri-N-acetylgalacto-samine (PEI-PEG-triGAL) derivatives. In one embodiment, the nucleic acid molecules of the invention are formulated as described in U.S. Patent Application Publication No. 20030077829, incorporated by reference herein in its entirety.

In one embodiment, a siNA molecule is complexed with membrane disruptive agents such as those described in US 2001/0007666, incorporated by reference herein in its entirety. In another embodiment, the membrane disruptive agent or agents and the siNA molecule are also complexed with a cationic lipid or helper lipid molecule, such as those lipids described in U.S. Pat. No. 6,235,310, incorporated by reference herein in its entirety. In one embodiment, a siNA molecule is complexed with delivery systems as described in US 2003/077829, WO 00/03683 and WO 02/087541, each incorporated herein by reference.

Alternatively, certain siNA molecules of the instant invention can be expressed within cells from eukaryotic promoters (e.g., Izant and Weintraub, 1985, Science, 229, 345; McGarry and Lindquist, 1986, Proc. Natl. Acad. Sci., USA 83, 399; Scanlon et al., 1991, Proc. Natl. Acad. Sci. USA, 88, 10591-5; Kashani-Sabet et al., 1992, Antisense Res. Dev., 2, 3-15; Dropulic et al., 1992, J. Virol., 66, 1432-41; Weerasinghe et al., 1991, J. Virol., 65, 5531-4; Ojwang et al., 1992, Proc. Natl. Acad. Sci. USA, 89, 10802-6; Chen et al., 1992, Nucleic Acids Res., 20, 4581-9; Sarver et al., 1990 Science, 247, 1222-1225; Thompson et al., 1995, Nucleic Acids Res., 23, 2259; Good et al., 1997, Gene Therapy, 4, 45. Those skilled in the art realize that any nucleic acid can be expressed in eukaryotic cells from the appropriate DNA/RNA vector. The activity of such nucleic acids can be augmented by their release from the primary transcript by a enzymatic nucleic acid (Draper et al., PCT WO 93/23569, and Sullivan et al., PCT WO 94/02595; Ohkawa et al., 1992, Nucleic Acids Symp. Ser., 27, 15-6; Taira et al., 1991, Nucleic Acids Res., 19, 5125-30; Ventura et al., 1993, Nucleic Acids Res., 21, 3249-55; Chowrira et al., 1994, J. Biol. Chem., 269, 25856.

Where the siNA is an RNA molecule, the siNA can be expressed from transcription units inserted into a vector. The recombinant vectors can be DNA plasmids, non-viral vectors or viral vectors. siNA expressing viral vectors can be constructed based on, but not limited to, adeno-associated virus, retrovirus, adenovirus, or alphavirus. The recombinant vectors capable of expressing the siNA molecules can be delivered as described above, and provide for transient or stable expression. For example, such vectors can include: 1) a transcription initiation region; 2) optionally, a transcription termination region; and 3) a nucleic acid sequence encoding at least one strand of an siNA molecule, wherein the sequence is operably linked to the initiation region and the termination region in a manner that allows expression and/or delivery of the siNA molecule.

Subject Amenable to Therapy

Agents that inhibit cellular proliferation (e.g., through inhibition of cyclin D1 phosphorylation and/or ubiquitination) are useful in treatment of any suitable cellular proliferative disease associated with cyclin D1-mediated aberrations in cell cycling, e.g., overexpression of cyclin D1. Several cancers have been characterized as having elevated cyclin D1 expression and/or elevated cyclin D1 degradation which mediates a tumorigenic phenotype. As discussed in the Examples below, elevated cyclin D1 degradation in a cancerous cell relative to a normal cell of the same tissue type indicates that tumorigenesis is mediated by cyclin D1 degradation, and thus the cancer is amenable to treatment by inhibition of cyclin D1 degradation (e.g., by inhibition of cyclin D1 phosphorylation by MAPK and/or inhibiton of ubiquitination of cyclin D1 by an FBXW8-containin E3 ligase.

Exemplary cancers include: breast cancer (e.g., carcinoma in situ (e.g., ductal carcinoma in situ), estrogen receptor (ER)-positive breast cancer, ER-negative breast cancer, breast cancers having a mutant BRCA1 allele or other forms and/or stages of breast cancer); lung cancer (e.g., small cell carcinoma, non-small cell carcinoma, mesothelioma, and other forms and/or stages of lung cancer); colon cancer (e.g., adenomatous polyp, colorectal carcinoma, and other forms and/or stages of colon cancer); ovarian cancer; endometrial cancer; oral cancers (e.g., oral squamous cell carcinomas); squamous cell carcinoma of the head and neck; liver cancer (e.g., hepatitis-related liver cancer); pancreatic cancer; esophageal carcinoma; laryngeal cancer; leukemias; lymphomas, neural cancers; and rhabdoid tumors.

Subjects suspected of having a cancer associated with aberrant cyclin D1 degradation can be screened prior to therapy. Further, subjects receiving therapy may be tested in order to assay the activity and efficacy of the agent administered, e.g., the siNA of FBXW8, CUL1, and/or CUL7. Significant improvements in one or more of parameters is indicative of efficacy. It is well within the skill of the ordinary healthcare worker (e.g., clinician) to adjust dosage regimen and dose amounts to provide for optimal benefit to the patient according to a variety of factors (e.g., patient-dependent factors such as the severity of the disease and the like, the compound administered, and the like).

Kits

Kits with unit doses of the subject compounds, usually in topical, oral or injectable doses, are provided. In such kits, in addition to the containers containing the unit doses will be an informational package insert describing the use and attendant benefits of the drugs in treating pathological condition of interest. Representative compounds and unit doses are those described herein above.

In one embodiment, the kit comprises components for carrying out the in vitro assays or in vivo assays described above. In other embodiments, the kit comprises an siNA formulation in a sterile vial or in a syringe, which formulation can be suitable for injection in a mammal, particularly a human.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

Methods and Materials

The following methods and materials are used in the examples below.

Chemicals, Cell culture, Establishment of Inducible Cell Lines and Cell Cycle Analysis. The proteasome inhibitor MG132 (Calbiochem), MEK inhibitor U0126 (Promega), CDK4 inhibitor AG12275, GSK3 inhibitor BIO (Calbiochem), ecdysone analog Ponasterone A (Invitrogen), 4-hydroxytamoxifen (Sigma) cyclohexamide (Sigma) were suspended in DMSO. Leptomycin B (Calbiochem) was resolved in 70% methanol. The GSK3 inhibitor LiCl and thymidine (Gibco) were suspended in distilled filtered water or PBS.

HCT 116, SW480, T98G, CCD841 CoN, WI-38, NIH 3T3, U-2 OS, and HEK293 cells were obtained from the American Type Culture Collection. ΔB-Raf:ER^{TAM} (ER-BRAF) NIH 3T3 cells are available in the art (see, e.g., Woods et al. Mol Cell Biol. 2001 May;21(9):3192-3205 and Pritchard et al. Mol Cell Biol. 1995 November;15(11):6430-6442). Ecdysone-inducible cell lines were established using the ecdysone-inducible mammalian expression system (Invitrogen). pIND-inducible expression vector resistant to Hygromycin B, which contains HA-tagged cyclin D1 T286A, was transfected by using FuGENE6 (Roche) upon HCT 116 cells carrying ecdysone response receptor. One hundred fifty single-cell derived independent drug-resistant colonies were cloned and screened for exogenous expression.

Cell cycle analysis was carried out as described previously (Tetsu et al. (1999) Nature 398: 422-6; Tetsu et al. (2003) Cancer Cell 3: 233-45.

Vectors, Site-directed Mutagenesis and Retroviral Gene Expression. CMV-HA tagged ubiquitin, pcDNA3 HPV16-E7, and pSG5 H-Ras V12 are available in the art (see, e.g., Aberle et al. EMBO 1997; 16(13):3797-3804; Smola-Hess et al. J. Gen Virol 2005; 86:1291-1296;and Rodriguez-Viciana et al. Cell 1997 May;89(3):457-467.

CUL1 expression vectors and CKS1 expression vectors are available in the art (see, e.g., Piva et al. Mol Cell Biol. 2002 December;22(23):8375-87 and Kitajima et al. Am J Pathol. 2004 December;165(6):2147-55).

CMV-Flag tagged CUL7 DNA plasmids are available in the art (see, e.g., Dias et al. Proc Natl Acad Sci USA. 2002 Dec. 24;99(26):16601-6).

pcDNA3 cyclin D1 T286A, cyclin D1 ΔD mutants, and F-box deletion (ΔF) mutant form of FBXW8 or SKP2, pcDNA3 MEK1 ΔN3/S218D/S222D and MEK1 K97M/S218A/S222A were generated by using site-directed mutagenesis according to the manufacturer's instructions (QuickChange and ExSite, Stratagene). Cyclin D1 T286A, FBXW8, ΔF FBXW8, ΔF SKP2 cDNA fragments was subcloned into pFB retrovirus expression vector or pFB-Neo retrovirus expression vectors (Stratagene). Transfection was carried out using amphotropic phoenix cells. Supernatant was harvested 48-72 hr after transfection, filtered, and stored at −80° C. Cells were infected with a virus media containing 8 (g/ml polybrene for 4 hours, subsequently replaced with a fresh media and cultured for further 48 hours.

Small Interfering (si) RNAs. The following FBXW8, SKP1, CUL1, and CUL7 siRNA oligonucleotides target sites were selected to knockdown endogenous expression:

FBXW8
(AAGAUGUGCACAGGUGAGCAA),
CUL1
(AAUAGACAUUGGGUUCGCCGU),
and
CUL7
(AAGGAUGAGAUCUAUGCCAAC).

Mismatch oligonucleotides for FBXW8, CUL1, and CUL7 are 8 bp nucleotides different from their target sequences respectively. Commercially available siRNAs for SKP1 (SMART pool, Dharmacon) were used. siRNAs were transfected by using Oligofectamine or Lipofectamine (Gibco, Invitrogen). Relative gene expression following siRNA treatment was measured by a real-time quantative RT-PCR analysis performed by the UCSF Cancer Center Genome Core Facility using the TaqMan assay (Applied Biosystems).

Transformation Assay in NIH 3T3 Cells. Low passage NIH 3T3 cells were seeded in 6-well dishes the day before transfection. Cells were transfected either with 40ng of pSG5 H-Ras V12, 1 (g of empty vector or cyclin D1 T286A using Lipofectamine (Invitrogen). Forty-eight hours later, the cells were trypsinized and re-plated into 100 mm dishes. After reaching confluence, the cells were kept for two weeks in DME media containing 5% calf serum, after which they were fixed with 100% methanol, and stained with Giemsa solution.

Immunofluorescence Analysis. Cultured cells on multiwell chamber slides (Nalge Nunc) were fixed with 4% paraformaldehyde in PBS and permeabilized in PBS containing 0.1% Triton X-100. Primary antibodies were diluted 1:100 in PBS containing 5% normal goat serum and applied for 2 hours. Proteins were detected either with mouse monoclonal antibodies or rabbit polyclonal antibodies followed by fluorescent substrate conjugated anti-mouse or anti-rabbit secondary antibody (Molecular Probes). For example, Cyclin D1 was detected either with mouse monoclonal cyclin D1 antibody (A-12, Santa Cruz) followed by fluorescent substrate conjugated anti-mouse or anti-rabbit secondary antibody (Molecular Probes). Nuclei were visualized using Hoechst 33258 (Molecular Probes). Fluorescence image was detected using LEICADMRD microscope (Leica).

Immunoblotting Analysis. Total protein was prepared as described previously (Tetsu and McCormick, 2003). NE-PER nuclear and cytoplasmic extraction reagents (Pierce) were used for nuclear and cytoplasmic fractionation. SDS-PAGE was described previously (Tetsu and McCormick, 2003). Western blots were developed by enhanced chemiluminescence (Amersham or Upstate). The following monoclonal and polyclonal primary and secondary antibodies were used: cyclin D1 (A-12, M-20, Santa Cruz), cyclin A (Transduction, C-19, Santa Cruz), cyclin D3 (Transduction), cyclin E (Ab-1, Calbiochem), p21 Cip1 (Transduction), p27 Kip1 (Transduction), ERK1/2 (Transduction or Promega), phospho-ERK1/2 (E-4, Santa Cruz), CDK4 (Transduction, or H-303, Santa Cruz), CDK6 (C-21, Santa Cruz), SKP1 (55893, PharMingen), CUL1 (ZL18, Zymed), CUL7 (BL653, Bethyl Laboratories), RBX1 (Ab-1, NeoMarkers), Ubiquitin (P4D1, Santa Cruz), GFP (FL, Santa Cruz), Rb (4H1, Cell Signaling), phospho-Rb on Ser780 and Ser795 (CeU Signaling), MEK1 (Transduction), Histone HI (AE-4, Santa Cruz), β-actin (Sigma), HA (12CA5, Roche), Flag (M2, Sigma), V5 (Invitrogen), p107 (C-18, Santa Cruz), p130 (C-20, Santa Cruz), E2F1 (Transduction), E2F2 (C-20, Santa Cruz), E2F3 (C-18, Santa Cruz), E2F4 (C-20, Santa Cruz), E2F5 (MH-5, Santa Cruz), Cdc6 (H-304, Santa Cruz), MCM3 (Abeam), GSK3β (Transduction), phospho-GSK3 (5G-2F, Upstate), ERK1/2 (Transduction or Promega), phospho-ERK1/2 (E-4, Santa Cruz), GFP (FL, Santa Cruz), V5 (Invitrogen), Sheep anti-mouse IgG HRP and Donkey anti-rabbit IgG HRP (Amersham, Roche). Intensities of bands were quantified using Gel Doc 600 and Quantity One software (BioRad).

Generation of a Cyclin D1 Phosphorylation Specific Antibody. Phospho-specific antibody against Thr286 of cyclin D1 was raised using KLH-conjugated phospho-peptide KDLAC-pT-PTDVR as an antigen in collaboration with Zymed Inc. Rabbits were immunized three times with the peptides and serum was collected at 3 months, and followed by affinity-purification using affinity gel coupled with phosphorylated peptide. Anti-nonphosphorylated cyclin D1 antibodies were eliminated by the affinity-absorption using gel coupled with unphosphorylated peptide (Zymed).

Immunoprecipitation and Immunoblotting Analysis. Immunoprecipitation and immunoblotting analysis was carried out as described previously (Tetsu and McCormick, 2003). Following antibodies were used for immunoprecipitation; Flag (M2 Agarose-conjugated, Sigma), cyclin D1 (A-12, Agarose-conjugated, Santa Cruz), CDK4 (H-303 or C-22 Agarose-conjugated, Santa Cruz), CDK6 (C-21, Santa Cruz), FLA (M2 Agarose-conjugated, Sigma), and HA (Y-11 Agarose-conjugated, Santa Cruz). Immunoblotting was performed using antibodies described above.

Generation of GST-fusion Proteins. Full-length WT cyclin D1, full-length T286A cyclin D1 mutant, or the ΔD C-terminal 131 residues of cyclin D1 were cloned into pET-42 vector (Novagen) respectively to generate in-frame GST-cyclin D1 fusion proteins. Plasmid DNA was transformed using One Shot BL21 (DE3) pLysS competent cells (Invitrogen). Fresh bacteria colonies were selected and cultured in LB medium to reach exponentially growing phase and then induced by the addition of 1 mM of isopropyl β-D-thiogalactopyranoside (IPTG) to express recombinant proteins. Bacteria were lysed in BugBuster protein extraction reagent (Novagen) containing 1 μl/ml Benzonase following repeated cycles of manipulation by freezing and thawing. GST-fusion proteins were absorbed to G1 utathione Sepharose 4B columns (Pharmacia) and then eluted with 50 mM Tris-HCl (pH 8.0) elution buffer containing 10 mM G1 utathione.

InVitro Kinase Assay. GST-cyclin D1 or GST-Rb (Santa Cruz) fusion proteins were used for in vitro kinase assays. Reactions were performed with the kinase buffer 50 mM Tris-HCl (pH 8.0) and 1 mM DTT containing 30 mM ATP and 10 μCi of γ-³²P ATP in the presence of 10 ng of recombinant MEK1 activated GST-ERK2 (14-550, Upstate) or CDK4 immune-complexes from cultured cells at 30° C. for 30 min. Reactions were stopped by adding sample loading buffer. Samples were separated with SDS-PAGE and then ³²P uptake was detected by autoradiography.

In Vitro Ubiquitination Assay. GST-full length wild-type cyclin D1 (CD1 WT), cyclin D1 T286A, or ΔD cyclin D1 fusion protein (100 ng) was mixed with HeLa cell extracts Fraction II (BostonBiochem), Ubiquitin (BostonBiochem), Ubiquitin Aldehyde (BostonBiochem), the proteasome inhibitor MG132 and ATP-regenerating system (BostonBiochem) either with or without 10 ng of recombinant active ERK2 (14-550, Upstate) in final volume of 20 μl. Reactions were performed at 37° C. for 2 hr and then terminated by boiling for 5 min with SDS sample buffer. Samples were separated by SDS-PAGE and immunoblotted with a cyclin D1 antibody.

In other assays, GST-full length cyclin D1 WT, T286A mutant fusion protein (100 ng) was mixed with each in vitro translated F-box protein with Fraction II cell extracts with ATP, Ubiquitin, and in vitro-translated either SKP1, RBX1 and CUL1, or SKP1, RBX1 and CUL7 proteins in the presence or absence of 10 ng of recombinant active ERK2 (14-550, Upstate) in a final volume of 20 μl. Reactions were performed at 30° C. for 2 hr and then terminated by boiling for 5 min with SDS sample buffer. Samples were separated by SDS-PAGE and immunoblotted with a cyclin D1 antibody.

Reconstitution of Cyclin D1 Polyubiquitination In Vitro. Recombinant SCFL^{FBXW8} were prepared from transfected HEK293 cells. Equal amounts of the SCFL^{FBXW8} immune-complexes were mixed with 1 μg GST-full-length CD1 WT protein in the presence of 30 ng recombinant active ERK2 (14-550, Upstate) and 0.5 mM ATP for 30 min on ice to allow binding. To the mixture was added 50 ng E1 (BostonBiochem), 100 ng E2 (UbcH5c, BostonBiochem), 2 μg ubiquitin (BostonBiochem), and 1 μg ubiquitin aldehyde (BostonBiochem). Reactions were performed with a buffer containing 50 mM Tris-HCl (pH 8.0), 1 mM DTT, 5 mM MgCl₂, 0.5 mM EDTA, 1.5 mM ATP in the presence of 10% glycerol at 30° C. for 1 hour and then terminated by boiling for 5 min with SDS sample loading buffer. Samples were separated by SDS-PAGE and immunoblotted with a cyclin D1 antibody (A-12, Santa Cruz).

Pulse-Chase Analysis. Cells were pulse-labeled with ³⁵S-methionine for an hour, chased with cold methionine for the indicated times, and then lysed. Cyclin D1 was immunoprecipitated and then analyzed with SDS-PAGE. Levels of metabolically labeled-cyclin D1 were estimated by quantitative scanning using the Quantity One (Bio-Rad) software and blotted on the graph to determine the half-life of cyclin D1.

Real-Time Quantitative RT-PCR Analysis. Total RNA was isolated using TRizol reagent (Invitrogen). iSCRIPT (Biorad) was used for cDNA synthesis. Pre-designed PCR primers and probes for CCNA2, CDC6, and MCM3 were purchased from Applied Biosystems. Real time quantitative RT-PCR analyses were performed by the UCSF Cancer Center Genome Core Facility using the TaqMan assay chemistry (Applied Biosystems).

### Example 1

### Cyclin D1 Protein is Destabilized Specifically in S Phase in Cancer Cells

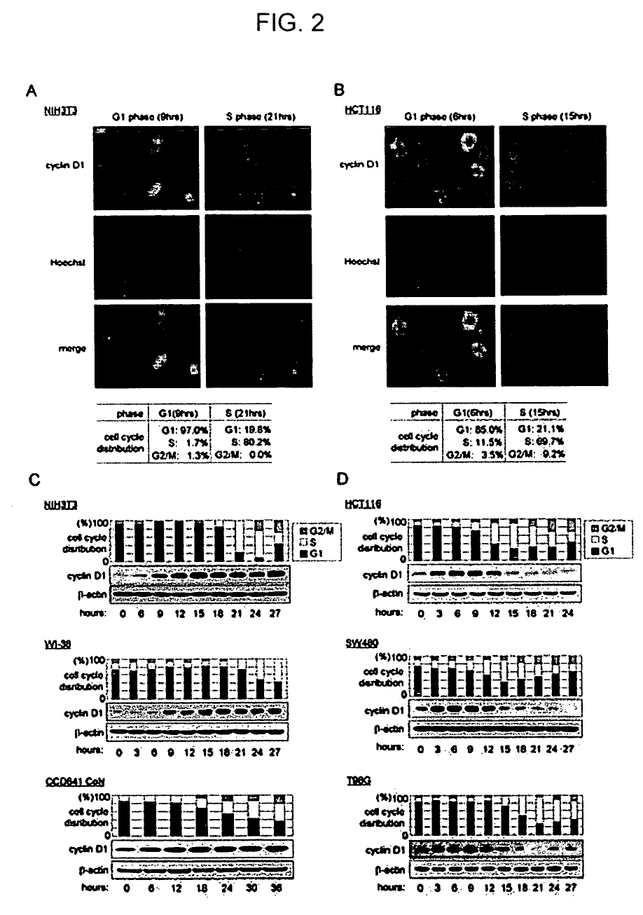

In order to examine the contribution of cyclin D1 to cell cycle in cancer cells, the subcellular distribution of endogenous cyclin D1 throughout the cell cycle in cancer cells were assessed in NIH 3T3 mouse fibroblast cells and HCT 116 colon cancer cells (FIG. 2, Panels A and B). Cells were rendered quiescent by serum starvation for 48 hours and then stimulated with the addition of 10% FBS containing media to allow synchronous progression. Cell cycle profiles were determined by flow-cytometric cell cycle analysis (FIG. 2, Panels A and B, bottom tables) and cyclin D1 was visualized in fixed cells by immunofluorescence microscopy. In both NIH 3T3 and HCT 116 cells, cyclin D1 was expressed in the nucleus during the G1 phase, and relocalized to the cytoplasm as cells proceeded into the S phase (FIG. 2, Panels A and B). However, the HCT 116 cells showed much lower expression of cyclin D1 during the S phase than in the G1 phase.

The expression profile of cyclin D1 protein during cell cycle progression from quiescence was examined in order to determine whether degradation of cyclin D1 protein is accelerated during the S phase in cancer cells. Three normal cell lines; NIH 3T3 mouse and WI-38 human fibroblasts, and CCD841 CoN normal colon epithelium cells and three cancer cell lines; HCT 116 and SW480 colon cancers and T98G glioblastomas (FIG. 2, Panels C and D) were released from quiescence at the G0/G1 phase. The cell cycle profiles were determined by flow-cytometric cell cycle analyses. In both normal and cancer cells, expression of cyclin D1 gradually increased after re-entry into the cell cycle and reached its maximum at the G1-S transition. In all three normal cells, levels of cyclin D1 remained constant during the S phase (FIG. 2, Panel C). In contrast, all three cancer cells showed a dramatic reduction of cyclin D1 expression during S phase (FIG. 2, Panel D). Similar data was obtained from U-2 OS osteosarcoma cells (data not shown). These results demonstrate that cyclin D1 protein turnover is accelerated during the S phase in cancer cells.

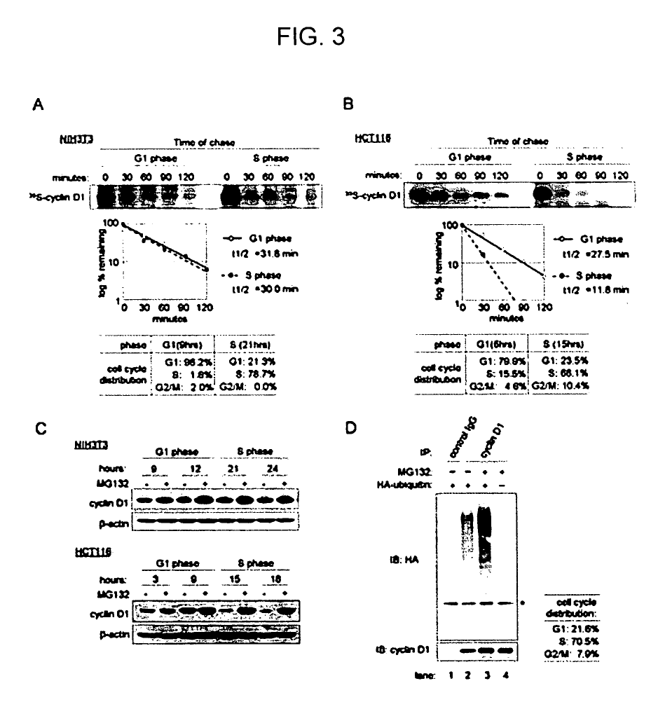

NIH 3T3 mouse fibroblast and HCT 116 colon cancer cells were synchronized at G0/G1 phase and released from quiescence in order to confirm that cyclin D1 protein turnover is accelerated during the S phase in cancer cells. At 9 (NIH 3T3) or 6 (HCT 116) hrs when some of the cells were in the G1 phase and at 21 (NIH 3T3) and 15 (HCT 116) hrs when the majority of the cells were in S phase (FIG. 3, Panels A and B, bottom tables), pulse-chase analyses were performed on metabolically labeled-cyclin D1 protein (FIG. 3, Panels A and B). Levels of metabolically labeled-cyclin D1 were estimated by quantitative scanning using the Quantity One (Bio-Rad) software (FIG. 3, Panels A and B, bottom graphs). In HCT 116 cells, the half-life of cyclin D1 protein in the S phase was reduced (T1/2=11.8 min) from the G1 phase (T1/2=27.5 min). In contrast, there was no difference in the half-life between the G1 and the S phase in the NIH 3T3 cells. These results demonstrate that cyclin D1 protein is destabilized specifically in S phase in cancer cells.

### Example 2

### Cyclin D1 Protein is Degraded During the S Phase Through the Ubiquitin-proteasome Pathway in Cancer Cells

HCT 116 and NIH 3T3 cells were treated with the proteasome inhibitor MG132 at each time point during cell cycle progression (FIG. 3, Panel C). In HCT 116 colon cancer cells, cyclin D1 protein accumulated significantly in S phase, although there was no significant accumulation during the G1 phase. In contrast, there was no difference between the G1 and S phase in NIH 3T3 cells. The experiment was repeated with SW480 colon cancer and T98G glioblastoma cells which resulted in a similar profile to the HCT 116 cells (data not shown). In contrast, there was no remarkable different between the G1 and S phase in WI-38 cells, a human diploid cell line derived from normal embryonic β months gestation) lung tissue. These results demonstrate that cyclin D1 is destabilized during the S phase through the 26S proteasome pathway in cancer cells.

To confirm that the destabilization of cyclin D1 in the S phase is related to polyubiquitination, HCT 116 colon cancer cells were transfected with (lanes 1-3) or without (lane 4) HA-tagged ubiquitin cDNA and then synchronized to the S phase (FIG. 3, Panel D). Cells were treated with (lanes 3 and 4) or without (lanes 1 and 2) the proteasome inhibitor MG132 for an hour. Lysates were immunoprecipitated with either a cyclin D1 antibody (lanes 2-4) or a control IgG (lane 1) and immunoblotted with a HA antibody (FIG. 3, Panel D). A group of slower migrating bands was detected by the HA antibody exclusively in the anti-cyclin D1 immunoprecipitates in the presence of ubiquitin (lane 2 and 3). The reduced mobility bands were enhanced further after exposure to MG132 (lane 3), indicating that these bands included polyubiquitinated cyclin D1. These results demonstrate that cyclin D1 protein is degraded during the S phase through the ubiquitin-proteasome pathway in cancer cells.

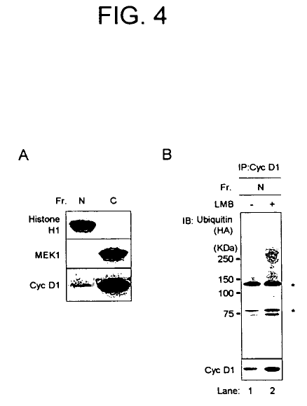

To determine the cellular fraction where cyclin D1 degradation is accelerated during S phase, we extracted nuclear (N) and cytoplasmic (C) protein from cell lysates. Histone HI was exclusively detected in the nuclear fraction, whereas MEK1 totally expressed in the cytoplasmic extract, suggesting that we successfully fractionated cell lysates (FIG. 4, Panel A). As we observed above, the majority of cyclin D1 was localized in the cytoplasm (FIG. 4, Panel A). Nuclear and cytoplasmic extracts were immunoprecipitated with antibodies to cyclin D1 (lanes 1 and 2) or IgG (lane 3) and immunoblotted with a HA antibody. Because polyubiquitinated cyclin D1 bands were predominantly detected in the cytoplasmic extracts and because inhibition of nuclear-to cytoplasmic localization of cyclin D1 through Leptomycin B (LMB) did not enhance these bands significantly in the nucleus (FIG. 4, Panel B), we concluded that cyclin D1 protein is degraded in the cytoplasm specifically in S phase by a proteasome-dependent mechanism in cancer cells.

### Example 3

### Expression of Cyclin D1 Protein in the Nucleus Decreases Through the G1-S Transition in Tumor Cells

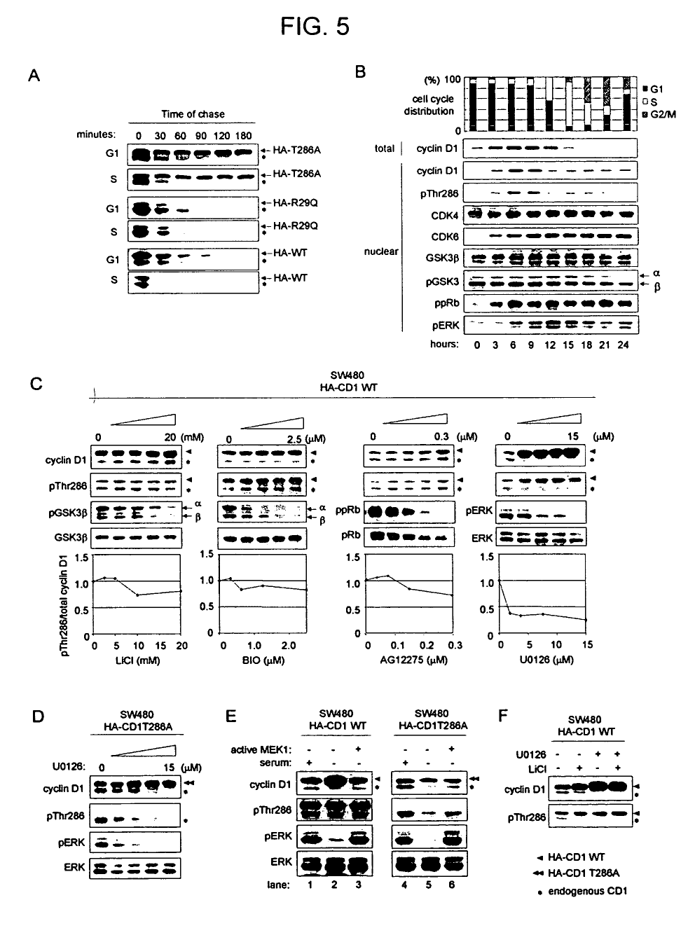

Nuclear proteins were fractioned from HCT 116 colon cancer cells to assess expression levels of cyclin D1 and its catalytic partners. HCT 116 colon cancer cells were synchronized at the G0/G1 phase by serum starvation and stimulated with an addition of 10% FBS containing media to induce re-entry into the cell cycle (FIG. 5, Panel B). Cyclin D1 expression was reduced in the nucleus at 12 hrs, which was 3 hrs earlier than detected in total cell lysates. Cell cycle analysis demonstrated that the 12 hr point corresponded to the G1-S transition. These findings demonstrate that cyclin D1 proteolysis is necessary for G1-S transition in tumorigenic cells. The same membrane was re-blotted with the phosphorylation-specific antibody for cyclin D1 Thr286. The peak of expression appeared at 9 hrs just before a decrease of cyclin D1 expression, demonstrating that phosphorylation-dependent cyclin D1 protein turnover in the nucleus is accelerated during G1-S transition in tumorigenic cells. In contrast, CDK4 and CDK6, the catalytic partners of cyclin D1, showed little change throughout a complete cycle, demonstrating that CDK4 and CDK6 activities are regulated by expression of cyclin D1 (FIG. 5, Panel B)

### Example 4

### Phosphorylation of Cyclin D1 Protein is Mediated by the Same Mechanism as Cyclin D1 Gene Expression, V_{1A} the MAPK Signaling Pathway

As illustrated in Example 1, degradation of cyclin D1 protein is accelerated during the S phase in T98G glioblastoma. T98G cells contain mutations in PTEN, which confer inhibition of GSK3β. A recent report questioned the role of GSK3β for cyclin D1 phosphorylation. The Ras pathway activates P13K/PKB/Akt kinases, which in turn inhibit GSK3β: therefore Ras should stabilize cyclin D1 protein (Diehl et al., 1998. Genes Dev. 12, 3499-511). However, Ras shows a completely opposite effect on cyclin D1 protein (Shao et al., 2000. J Biol Chem. 275, 22916-24). Ras signals facilitate cyclin D1 proteolysis but not stabilization which indicates that cyclin D1 turnover is independent of GSK3β and that GSK3β is probably not a major cyclin D1 kinase in vivo.

In order to investigate the role of GSK3β, immunoblot analysis was performed using a GSK3β antibody and a phosphorylation specific antibody to GSK3α and β. A phosphorylation specific antibody to GSK3α and β was used to measure their endogenous activities because phosphorylations of GSK3α at Tyr279 and GSK3β at Tyr216 are intramolecular autophosphorylation events in the cells and thereby phosphorylation status reflects their activities (Cole et al., 2004. Biochem J. 377, 249-55). There was no correlation between expression and activities of GSK3β and the cell cycle progression in tumorigenic cells because GSK3β and its phosphorylated form were ubiquitously expressed throughout the cell cycle and not linked to any specific phase of cell cycle in the examined cancer cells (FIG. 5, Panel B). The phosphorylation status of cyclin D1 protein was shown to be related to total cyclin D1 expression (FIG. 5, Panel B). These results indicate that cyclin D1 phosphorylation is mediated either by auto-phosphorylation through CDK4/6 kinases or by the same regulation mechanism as cyclin D1 gene expression via the MAPK signaling pathway.

The membrane was re-blotted with phosphorylation specific antibodies of Rb at Ser780 and p44 and p42 ERK1/2 at Thr202/Tyr204 respectively (FIG. 5, Panel B). Rb was phosphorylated throughout a complete cycle after re-entry into the cell cycle. In contrast, a dramatic induction of phosphorylated ERK1/2 in the nucleus was observed at 12 hrs and its gradual reduction after 12 hrs. Furthermore, there was an inverse correlation between cyclin D1 expression and ERK1/2 phosphorylation status, which indicates that phosphorylation of cyclin D1 protein can be mediated by the same regulation mechanism as cyclin D1 gene expression, via the MAPK signaling pathway.

### Example 5

### MAPK Regulates the Thr286 Phosphorylation of Cyclin D1 Protein In Vivo

To identify the kinase responsible for cyclin D1 Thr286 phosphorylation, small molecule inhibitors of GSK3β, CDK4, and MEK were used to determine whether they were able to alter the phosphorylation and stability of ectopically expressed WT cyclin D1 protein in cultured cells (FIG. 5, Panels C and D). Ectopically expressed WT cyclin D1 protein was used to assess the stability of cyclin D1 protein. The endogenous expression was not assessed because transcription of cyclin D1 is regulated by the Ras/MAPK signaling. Therefore, endogenous expression of cyclin D1 is unable to be detected following inhibition of MAPK activities through the MEK/MAPK inhibitors or serum starvation (Tetsu et al., 2003. Cancer Cell. 3, 233-45). Ectopic expression was distinguished from endogenous expression by the reduced mobility of the HA epitope tagged WT cyclin D1 protein as shown in FIG. 5, Panel A. The following inhibitors were used: LiCl and BIO for GSK3 inhibition (Meijer et al., 2003. Chem Biol. 10, 1255-66; Sato et al., 2004. Nat Med. 10, 55-63; Cohen et al., 2004. Nat Rev Drug Discov. 3, 479-87), AG12275 for CDK4 inhibition (Toogood, 2001. Med Res Rev 6, 487-98; Tetsu et al., 2003. Cancer Cell. 3, 233-45), and U0126 for MEK/MAPK inhibition (Favata et al., 1998. J. Bio. Chem. 273, 18623-32; Tetsu et al., 2003. Cancer Cell. 3, 233-45).

After 24 hours of treatment with these highly specific small molecule inhibitors, expression levels of cyclin D1 and its phosphorylated form were analyzed (FIG. 5, Panel C). Levels of phosphorylated cyclin D1 were estimated by quantitative scanning with Quantity One (Bio Rad) software and were normalized to levels of total cyclin D1 protein (FIG. 5, Panel C bottom graphs). The inhibition of the kinase activities were assessed using phosphorylation-specific antibodies for GSK3α/β, Rb and ERK1/2 in the same membrane (Cole et al., 2004. Biochem J. 377, 249-55; Kitagawa et al., 1996. EMBO J. 15, 7060-9; Tetsu et al., 2003. Cancer Cell. 3, 233-45). After 24 hours of treatment with the GSK3 inhibitors (LiCl or BIO) or the CDK4 inhibitor (AG12275), the phosphorylated forms of GSK3α/β and Rb had disappeared, indicating that the kinase activities of GSK3α/β or Rb were completely inhibited. However, there was no significant difference in both total cyclin D1 expression and its phosphorylated protein. Thus, the ratios of phosphorylated cyclin D1 protein at Thr286 to total cyclin D1 expression were not changed, which indicates that cyclin D1 phosphorylation was not mediated by GSK3α/β or CDK4 activities.

Cells were next treated with the MEK inhibitor U0126. Twenty-four hours after the exposure to U0126, phosphorylated ERK had disappeared, demonstrating that MAPK activities were totally inhibited. A dramatic induction of cyclin D1 expression was observed after U0126 treatment resulting in a significant reduction in the ratio although the phosphorylated cyclin D1 protein had not completely disappeared within the range of MEK/MAPK inhibitions through U0126. These results demonstrate that cyclin D1 protein is phosphorylated and destabilized by MAPK in cultured cells.

In order to rule-out the possibility that other phosphorylation sites within cyclin D1 protein might be involved with cyclin D1 stability, the cell line ectopically expressing cyclin D1 T286A was treated with U0126 (FIG. 5, Panel D). The ectopic expression of cyclin D1 T286A protein did not accumulate after drug treatment. These results indicate that MAPK-mediated cyclin D1 ubiquitination and degradation depend on Thr286 and not any other residue within the protein.

In order to determine whether the MEK inhibitor, U0126, inhibited other kinases that might be involve in cyclin D1 protein phosphorylation (Davies et al., 2000. Biochem J. 351, 95-105), endogenous MAPK activity was depleted by serum-starvation. Cell lines expressing either HA-WT or HA-T286A cyclin D1 (FIG. 5, Panel E) were tested. After 24 hours of serum depletion, MAPK activity was completely inhibited. Consequently, endogenous expression of cyclin D1 protein had significantly diminished. A dramatic induction of ectopically expressed WT cyclin D1 protein was observed without increasing the phosphorylated-cyclin D1 protein at Thr286. These results demonstrate that the majority of increased expression of cyclin D1 was not phosphorylated.

In contrast, there was no increase of ectopic expression of cyclin D1 T286A protein after serum starvation (FIG. 5, Panel E, lanes 1 and 2). To confirm WT cyclin D1 protein was accumulated through an inhibition of MAPK activities after serum depletion, an active form of MEK1 was transfected in the exponentially growing cells (FIG. 5, Panel E lane 3 and 6; Mansour et al., 1994. Science. 265, 966-70). After 24 hours after transfection, cells were serum-starved for an additional 24 hours. Panel E shows that phosphorylated-ERK, endogenous cyclin D1 and ectopic expression of WT were completely reversed. However, T286A cyclin D1 was not, which indicates that the effects of serum starvation were clearly via the Ras/MEK/MAPK signals.

To investigate whether the inhibition of MAPK activities would render these cells sensitive to GSK3 inhibition, HA-WT cyclin D1 SW480 cells were treated by combining U0126 with LiCl (FIG. 5, Panel F). The addition of LiCl resulted in little enhancement indicating that GSK3 does not contribute to phosphorylation and stability of cyclin D1 protein in vivo. Therefore, these results demonstrate that MAPK but not GSK3β is responsible for Thr286 phosphorylation.

### Example 6

### MAPK Ensures the Interaction with Cyclin D1 Protein Through the D-domain and Phosphorylates Thr286 of Cyclin D1

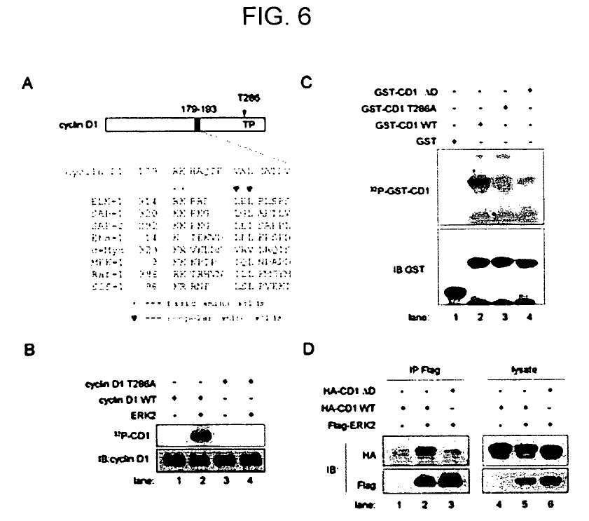

The cyclin D1 protein was searched for a D-domain using the Motif Scan software (http://scansite.mit.edu) because it is known that ERK/MAPK requires a kinase docking site (also known as a D-domain) on its substrate to increase the efficiency of phosphorylation (Sharrocks et al., 2000. Trends Biochem Sci. 25, 448-53). Through a series of searches, a highly stringent (within 0.041 percentile) D-domain in amino acids 179-193 of cyclin D1 protein (FIG. 6, Panel A) was identified, which indicates that the Ras/Raf/MEK/ERK MAPK signaling cascade is responsible for cyclin D1 phosphorylation.

In order to determine whether purified ERK/MAPK phosphorylates recombinant cyclin D1, p42 ERK2-associated GST-cyclin D1 in vitro kinase assays were performed (FIG. 6, Panels B and C). Kinase reactions performed in vitro demonstrated that purified ERK2 efficiently phosphorylated GST-full-length wild type (WT) cyclin D1 (FIG. 6, Panel B, lane 2). In contrast, ERK2 failed to phosphorylate the cyclin D1 mutant protein T286A (FIG. 6, Panel B, lane 4), indicating that Thr286 is the major phosphorylation site of ERK/MAPK. Identical results were obtained in the presence of purified CDK4 wherein ERK was shown to phosphorylate cyclin D1 at Thr286, not only in the monomeric form but also within CDK4-cyclin D1 complexes (data not shown).

To determine whether ERK/MAPK requires the D-domain for the efficient phosphorylation of cyclin D1 protein at Thr286, in vitro kinase assays were performed using a complete deletion of the D-domain (ΔD) from the GST-C-terminal cyclin D1 fusion protein that retains the biding site of MAPK. FIG. 6, Panel C shows that purified ERK2 effectively phosphorylated WT cyclin D1 (lane 2). However, this did not occur with the T286A (lane 3) and ΔD (lane 4) mutants. These results indicate that MAPK interacts with cyclin D1 protein through the D-domain to phosphorylate Thr286. Additionally, immunoprecipitation and immunoblotting analysis were performed following ectopic expression of Flag-tagged ERK2 with either HA-tagged WT or ΔD cyclin D1 in HCT 116 colon cancer cells (FIG. 6, Panel D). The ERK2 associated with WT cyclin D1 (lane 2) but associated poorly to ΔD cyclin D1 (lane 3). These experiments were repeated with SW480 colon carcinoma and T98G glioblastoma cells having similar results (data not shown).

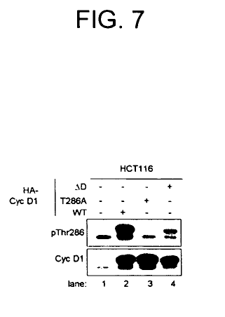

To establish the importance of MAPK on phosphorylation of cyclin D1 at Thr286 in cancer cells, various forms of cyclin D1 expression vectors were transfected into HCT 116 cells (FIG. 7). Ectopic expression of cyclin D1 was distinguished from endogenous expression by the reduced mobility of HA-tagged cyclin D1 protein. The phosphorylation status of exogenous cyclin D1 expression was analyzed at Thr286. The phosphorylation of cyclin D1 was significantly reduced by the deletion of the D-domain (FIG. 7, lane 4), which indicates that the majority of Thr286 phosphorylation occurs through MAPK activity.

### Example 7

### MAPK Regulates Stability of Cyclin D1 Protein

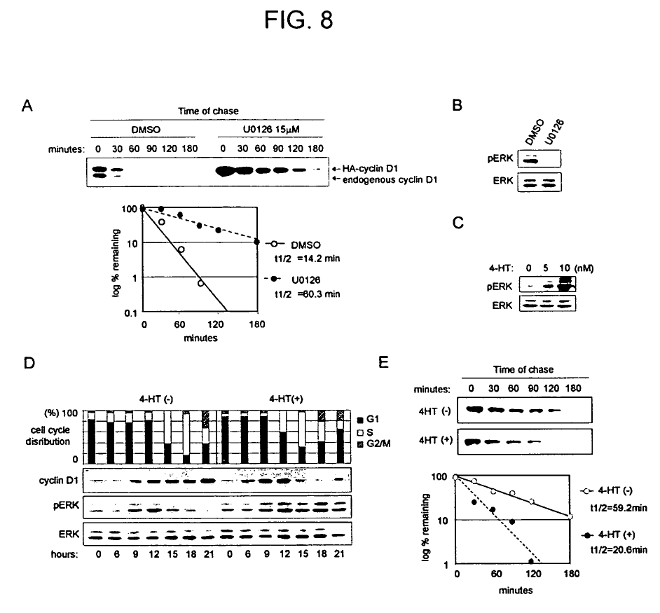

To determine whether accumulation of ectopically expressed WT cyclin D1 protein following MAPK inhibition was due to an increase of the protein stability, the half-life of ectopically expressed cyclin D1 protein was assessed following U0126 treatment (FIG. 8, Panels A and B). Exponentially growing HA-WT cyclin D1 SW480 cells were exposed to U0126 for 24 hours and subsequently treated with CHX and chased for 3 hours. Cells were harvested at different times and a protein blot was performed (FIG. 8, Panel A). FIG. 8, Panel B shows that MAPK activity was completely inhibited by U0126. Cyclin D1 expression was quantified and the half-life was calculated (FIG. 8, Panel A bottom graph). In U0126-treated cells, the half-life of cyclin D1 protein was extended (T1/2=60.3 min) from control DMSO-treated cells (T1/2=14.2 min). These results confirmed that the accumulation of WT cyclin D1 protein following MAPK inhibition was due to an increase of the protein stability which indicates that activation of the MAPK signals accelerates cyclin D1 proteolysis in tumorigenic cells.

NIH 3T3 cells stably expressing the ΔB-Raf:ER^{TAM} were next treated with 4-hydroxy-tamoxifen (4-HT) (FIG. 8, Panels C-E; Woods et al., 1997. Mol Cell Biol. 17, 5598-611; Ries et al., 2000. Cell. 103, 321-30. The addition of 10 nM 4-HT resulted in MAPK activation (FIG. 8, Panels C and D). The expression profile of cyclin D1 was examined during cell cycle progression from quiescence (FIG. 8, Panel D). Cells were serum starved for 48 hours and then stimulated by the addition of 10% FBS containing media with or without 4-HT. In the presence of 4-HT, a dramatic reduction of cyclin D1 expression was observed specifically in the S phase (FIG. 8, Panel D). In order to investigate whether this was due to accelerated turnover of the cyclin D1 protein, the half-life of endogenous expression of cyclin D1 protein (FIG. 8, Panel E) was assessed. Exponentially growing cells were cultured in the presence (+) or absence (−) of 10 nM 4-HT and subsequently treated with CHX and a half-life was calculated respectively. The half-life of cyclin D1 protein decreased significantly (T1/2=20.6 min) from control DMSO-treated cells (T1/2=59.2 min). These results demonstrate that MAPK regulates the stability of cyclin D1 protein and activation of the MAPK signals accelerates cyclin D1 proteolysis in tumorigenic cells.

### Example 8

### ERK/MAPK Phosphorylates Cyclin D1 at Thr286 In Vitro

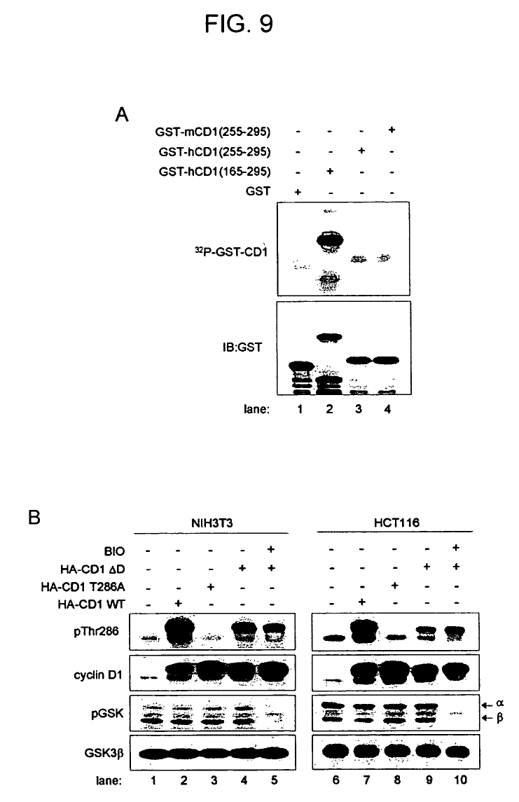

Purified MAPK or ERK was used to determine whether cyclin D1 is phosphorylated specifically at Thr286. A p42 ERK2-associated GST-cyclin D1 in vitro kinase assay was performed (FIG. 9, Panel A). The phosphorylation status of recombinant full length cyclin D1 was unable to be determined because the auto-phosphorylated form of ERK2 showed a similar mobility on SDS-PAGE. Therefore, an in-frame fusion protein between the carboxy (C)-terminal residues of cyclin D1 and GST was generated (FIG. 9, Panel A). It has become apparent that ERK requires a kinase docking site (also known as a D-domain) on its substrate to increase the efficiency of the phosphorylation (FIG. 6, Panel A and reviewed in Sharrocks et al., 2000). D-domains have been found in various ERK substrates such as Elk-1, Sap-1, Sap-2, Ets-1 and c-Myc (FIG. 6, Panel A; Bardwell et al., 2001. J Biol Chem. 276, 10374-86; Sharrocks et al., 2000. Trends Biochem Sci. 25, 448-53).

Two forms of GST-cyclin D1 fusions were tested as substrates (FIG. 9, Panel A lane 2-4). The GST-C-terminal 131 residues from 165 to 295 of cyclin D1 is the fusion protein that retains the biding site of MAPK (FIG. 6, Panel A, lane 2). The other form, GST-C-terminal 41 residues from 255 to 295 of cyclin D1, corresponds to the original fusion protein that GSK3β has been shown previously to markedly phosphorylate in vitro but does not have the D-domain of MAPK (FIG. 9, Panel A lane 3 and 4; Diehl et al., 1998. Genes Dev. 12, 3499-511). Kinase reactions performed in vitro demonstrated that purified ERK2 efficiently phosphorylated GST-cyclin D1 that retains the binding site of MAPK (FIG. 9, Panel A lane 2). In contrast, ERK2 failed to phosphorylate both human and mouse GST-C-terminal 41 residues of cyclin D1 proteins (FIG. 9, Panel A lane 3 and 4).

To establish the relative importance of MAPK on phosphorylation of cyclin D1 at Thr286 in vivo, various forms of cyclin D1 expression vectors were transfected in both NIH 3T3 mouse fibroblast and HCT 116 colon cancer cells (FIG. 9, Panel B). Ectopic expression of cyclin D1 was distinguished from endogenous expression by the reduced mobility of HA-tagged cyclin D1 protein. Phosphorylation status of exogenous cyclin D1 expression was analyzed at Thr286. Phosphorylation of cyclin D1 was dramatically reduced by the deletion of D-domain (lane 4 and 9 which indicates that the majority of Thr286 phosphorylation was through MAPK.

To determine the importance of GSK3β in the phosphorylation of cyclin D1 at Thr286, cells were treated with a highly specific GSK3β inhibitor BIO for an additional 24 hours following transfection of ΔD mutant form of cyclin D1 (FIG. 9, Panel B, lanes 5 and 10). Depletion of GSK3 kinase activities did not show any significant effect on the phosphorylation status of cyclin D1 at Thr286 in normal and cancer cells, although a slight change in NIH 3T3 cells was observed. These results demonstrate that MAPK is the major kinase for cyclin D1 phosphorylation at Thr286 and that Ras/MAPK-mediated phosphorylation of cyclin D1 protein followed by its protein ubiquitination and degradation is directly linked to an association of MAPK/ERK with cyclin D1.

### Example 9

### Ras/MAPK-Mediated Ubiquitination and Degradation of Cyclin D1 Protein is Directly Linked to the Association of MAPK/ERK with Cyclin D1

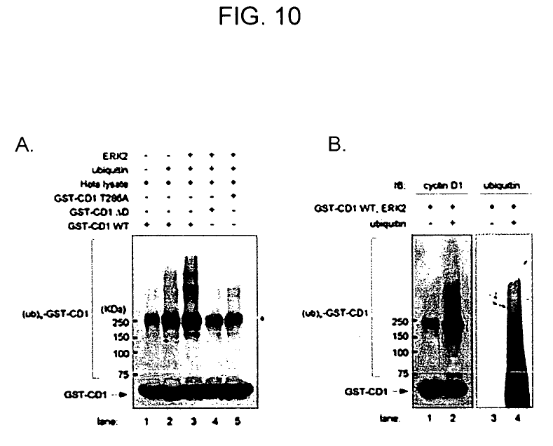

An ubiquitination assay was used to determine whether ubiquitination of cyclin D1 in vitro is required for MAPK-mediated phosphorylation of cyclin D1 protein (FIG. 10, Panels A and B). The ubiquitination assay system uses fraction II HeLa cell extracts as a source of the enzymes necessary to conjugate ubiquitin to substrates and ATP (Montagnoli et al., 1999. Genes Dev. 13, 1181-9). Ubiquitination of cyclin D1 was detected in an ubiquitin-dependent manner (FIG. 10, Panels A and B, lanes 1 and 2) in the presence of ATP (Diehl et al., 1997. Genes Dev. 11, 957-72).

The process was enhanced further by ERK2 (FIG. 10, Panel A, lane 3 and FIG. 10, Panel B, lanes 2 and 4). Slower migrating bands could not be detected in the absence of ubiquitin (FIG. 10, Panel B, lanes 1 and 3), indicating that these bands consist of polyubiquitinated forms of cyclin D1 (FIG. 10, Panel B, lanes 2 and 4). The ubiquitination was largely prevented in the D-domain deletion mutant form (ΔD) and the alanine for Thr286 substitution (T286A) of cyclin D1 (FIG. 10, Panel A, lanes 4 and 5). These results demonstrate that polyubiquitination requires the direct interaction of ERK2 with cyclin D1 and the phosphorylation of cyclin D1 at Thr286.

### Example 10

### Degradation of Cyclin D1 Protein Depends on Phosphorylation at Thr286 by ERK/MAPK

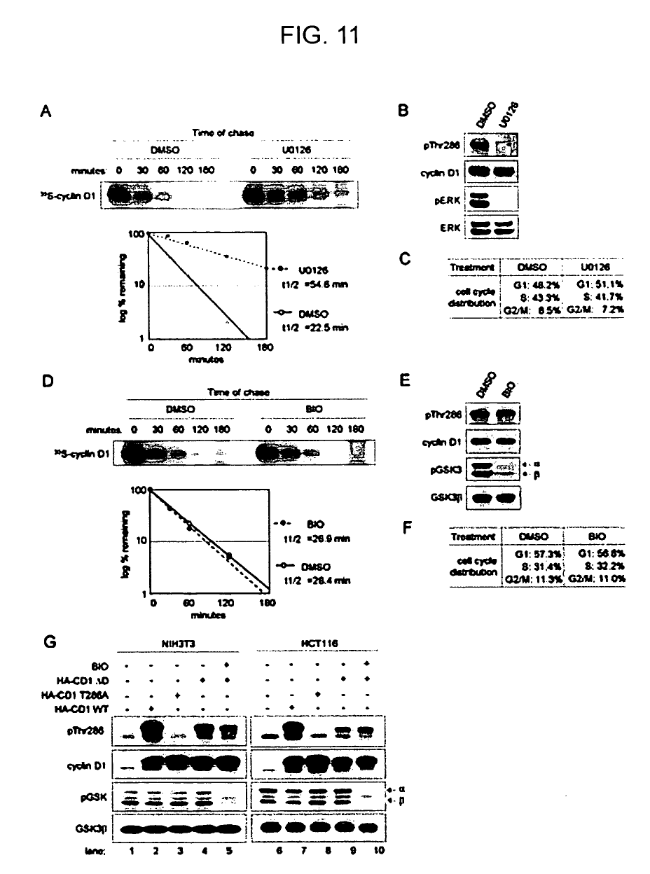

To determine the contribution of ERK/MAPK to the stability of cyclin D1 in cancer cells, MAPK activity was inhibited with the MEK inhibitor U0126 (Favata et al., 1998. J Bio Chem. 273, 18623-32; Davies et al., 2000. Biochem J. 351, 95-105). Exponentially growing HCT 116 colon cancer cells were treated with U0126 for 30 minutes (FIG. 11, Panels A-C). U0126 significantly depleted the phosphorylated form of ERK (pERK) from cultured cells, indicating that MEK had been completely inhibited (FIG. 11, Panel B) without affecting the cell cycle profile (FIG. 11, Panel C). This resulted in a dramatic reduction of phosphorylation of cyclin D1 at Thr286 (pThr286), although the level of total cyclin D1 was not changed through this short period of MEK/MAPK inhibition (FIG. 11, Panel B).

Pulse-chase analysis was performed on metabolically labeled-cyclin D1 protein after inhibition of MAPK activities (FIG. 11, Panel A). Levels of metabolically labeled-cyclin D1 were estimated by quantitative scanning using the Quantity One (Bio-Rad) software (Panel A, bottom graph). Reduction of MAPK activities led to an increase in the half-life of cyclin D1 protein from 22.5 min to 54.6 min. Similar observations were obtained from SW480 colon cancer cells (data not shown). These data indicate that phosphorylation and stability of cyclin D1 protein is regulated by ERK/MAPK activity.

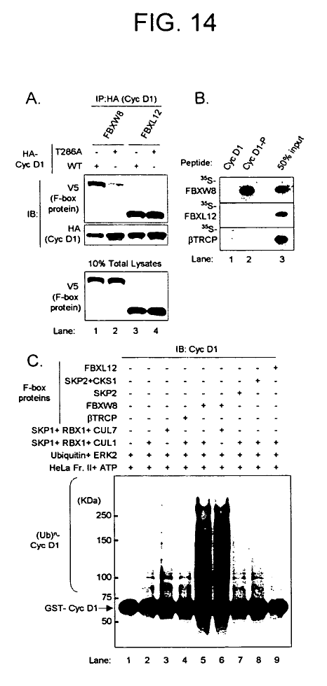

In contrast, no effect on phosphorylation status and stability of cyclin D1 protein through inhibition of GSK3α/β (FIG. 14, Panels D-F) were detected. GSK3α/β activities were inhibited through the highly selective GSK3α/β inhibitor BIO (Meijer et al., 2003. Chem Biol. 10, 1255-66; Sato et al., 2004. Nat Med. 10, 55-63; Cohen et al., 2004. Nat Rev Drug Discov. 3, 479-87). Cycling HCT 116 colon cancer cells were treated with BIO for 24 hours (FIG. 11, Panels D-F). A phosphorylation specific antibody to GSK3α and β was used to measure their endogenous activities because phosphorylations of GSK3α at Tyr279 and GSK3β at Tyr216 are intramolecular autophosphorylation events in the cells. Thus, phosphorylation status reflects their kinase activities (Cole et al., 2004. Biochem J. 377, 249-55).

After the treatment with BIO, phosphorylated forms of GSK3α/β disappeared (FIG. 11, Panel E), indicating that the kinase activities of GSK3α and β were completely inhibited without affecting the cell cycle profile (FIG. 11, Panel F). However, there was no significant difference in the expression of total cyclin D1, its phosphorylated protein or in the half-life of cyclin D1 protein (FIG. 11, Panels D and E). Similar data was obtained using another GSK3α/β inhibitor LiCl (data not shown). These results indicate that GSK3β does not play any significant role in the phosphorylation and stabilization of cyclin D1 protein in cancer cells.

To establish the importance of MAPK on phosphorylation of cyclin D1 at Thr286 in cancer cells, various forms of cyclin D1 expression vectors were transfected in both HCT 116 colon cancer and NIH 3T3 mouse fibroblast cells (FIG. 11, Panel G). Ectopic expression of cyclin D1 was distinguished from endogenous expression by the reduced mobility of HA-tagged cyclin D1 protein. The phosphorylation status of exogenous cyclin D1 expression was analyzed at Thr286. Phosphorylation of cyclin D1 was significantly reduced by the deletion of D-domain (FIG. 11, Panel G, lanes 4 and 9). This effect was dramatic in HCT 116 colon cancer cells which display sustained MAPK signaling (lane 9; Tetsu, et al., 2003. Cancer Cell. 233-45). These results indicate that the majority of Thr286 phosphorylation is through MAPK activity.

In order to determine the importance of GSK3β in the phosphorylation of cyclin D1 at Thr286, cells were treated with the GSK3 inhibitor BIO for 24 hours following transfection of the ΔD mutant form of cyclin D1 (FIG. 11, Panel G, lanes 5 and 10). A minimal effect on the phosphorylation status of cyclin D1 at Thr286 in HCT 116 cancer cells was observed after depletion of GSK3α/β kinase activity. Additionally, a reduction in NIH 3T3 cells was also observed. The inhibition of GSK3β kinase activity was tested to determine whether it affected localization of cyclin D1 in cancer cells (Alt et al., 2000. Genes Dev. 14, 3102-14).

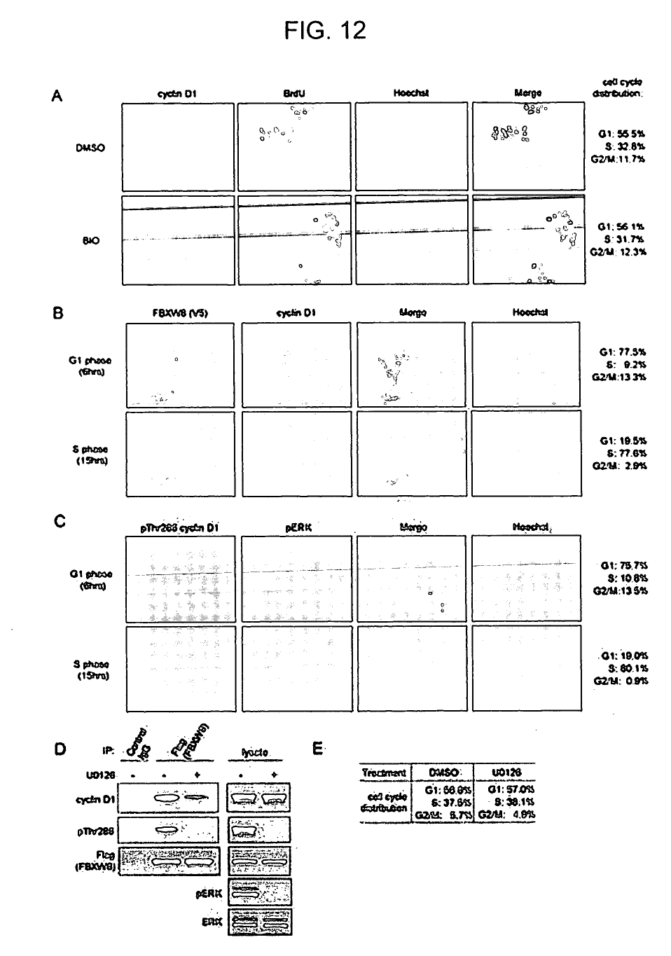

Exponentially growing HCT 116 colon cancer cells were treated with the GSK3 inhibitor BIO for 24 hours. After the treatment with BIO, the kinase activity of GSK3α/β was completely inhibited (see FIG. 11, Panel E) without affecting the cell cycle profile. To identify the cells in S phase, these cultured cells were pulse-labeled with thymidine analogue bromodeoxyuridine (BrdU) for an hour in the presence or absence of BIO. The subcellular distribution of cyclin D1 was visualized under the microscope (FIG. 12, Panel A). There was no significant difference in the subcellular localization of cyclin D1 following inhibition of GSK3 kinase activities. In both control and BIO-treated cells, a large proportion of S phase cells expressed cyclin D1 in the cytoplasm. In contrast, most of BrdU negative cells expressed cyclin D1 in the nucleus, indicating that these cells were probably in the G1 phase. The data indicates that GSK3β kinase activities are not necessary for MAPK-mediated cyclin D1 turnover in cancer cells. Therefore, the results demonstrate that MAPK is the major kinase for cyclin D1 phosphorylation at Thr286 and that Ras/MAPK-mediated phosphorylation of cyclin D1 protein followed by its protein ubiquitination and degradation is directly linked to an association of MAPK/ERK with cyclin D1 in cancer cells.

### Example 11

### Degradation of Cyclin D1 is Linked to an Increase in the Association of Cyclin D1 with the E3 Ligase Through the Enhanced Phosphorylation of Cyclin D1 by MAPK

HCT 116 colon cancer cells were transfected with V5 epitope-tagged FBXW8. Twenty-four hours later, cells were rendered quiescent by serum starvation and then stimulated with an addition of serum containing media to allow synchronous progression. Cell cycle profiles were determined by flow-cytometric cell cycle analyses. At various times, cells were fixed and we performed immunofluorescence with V5 epitope tag and cyclin D1 antibodies. The majority of FBXW8 was expressed in the cytoplasm throughout cell cycle. Colocalization of FBXW8 with cyclin D1 during S phase indicates that ubiquitination and subsequent degradation of cyclin D1 is accelerated in the cytoplasm as cells proceed into S phase. FBXW8 exclusively recognizes cyclin D1 protein in a phosphorylation-dependent manner and regulates the stability of cyclin D1 through the proteasome pathway (data not shown).

The subcellular localization of the FBXW8-containing E3 ligase and the phosphorylated forms of cyclin D1 and ERK/MAPK throughout the cell cycle in HCT 116 colon cancer cells (pThr286 cyclin D1 and pERK; FIG. 12, Panels B and C; Chen et al., 1992. Mol Cell Biol. 12, 915-27) were assessed in order to determine whether accelerated degradation of cyclin D1 could be linked to the increased association of cyclin D1 with the E3 ligase. Cells were synchronized and fixed as described above. Immunofluorescence was performed with phosphorylation specific antibodies to Thr286 cyclin D1 and ERK. This showed that phosphorylated cyclin D1 accumulated in the nucleus of HCT 116 cells in the G1 phase and was expressed in the cytoplasm during the S phase, which is a similar profile to its total expression. In contrast, HCT 116 colon cancer cells expressed a greater number of phosphorylated ERK in the cytoplasm throughout the cell cycle, although a lesser extent of activated form of ERK was detected in the nucleus. These results demonstrate that relocalization of cyclin D1 into the cytoplasm as cells proceed into S phase facilitate phosphorylation of cyclin D1 through ERK/MAPK.

FBXW8 DNA plasmid together with cyclin D1 and CDK4 expression vectors were transiently transfected in exponentially growing HCT 116 colon cancer cells. After 24 hours, cells were treated with U0126 to inhibit MEK/MAPK signaling for 30 minutes. Subsequently cells were collected and an immunoprecipitated-immunoblotting analysis was performed (FIG. 12, Panels D and E). U0126 significantly inhibited MAPK activities (pERK) and phosphorylation status of cyclin D1 at Thr286 (pThr286) without affecting the level of total cyclin D1 (FIG. 12, Panel D right panel) and cell cycle profile (FIG. 12, Panel E). This resulted in a significant decrease in the association of cyclin D1 with the E3 ligase FBXW8 (FIG. 12, Panel D left). These results demonstrated that accelerated cyclin D1 degradation is linked to an increase in the association of cyclin D1 with the E3 ligase through the enhanced phosphorylation of cyclin D1 via MAPK.

### Example 12

### The Stability of Cyclin D1 is Regulated Through the SCF or the SCF-like Pathway

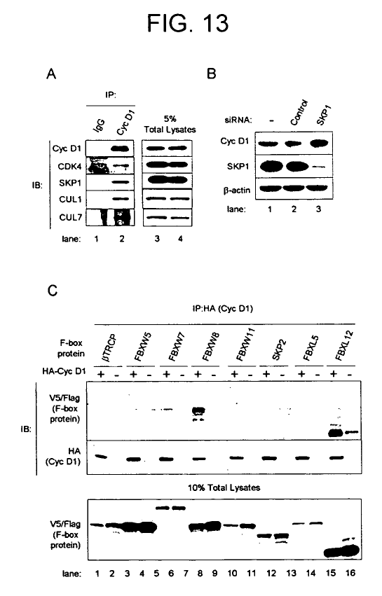

Immunoprecipitation-immunoblotting analysis was performed to determine whether cyclin D1 proteolysis is mediated by SCF or an SCF-like complex of E3 ligases in which an F-box protein determines the specificity of the substrate (FIG. 13, Panel A). Cyclin D1 from exponentially growing HCT 116 cells was immunoprecipitated and sequentially blotted with antibodies to cyclin D1, CDK4, SKP1, CUL1 and CUL7. Cyclin D1 was found to be associated with SKP1, CUL1 or CUL7, and CDK4, indicating that cyclin D1 proteolysis is mediated by the SCF (SKP1-CUL1-F-box protein) or the SCF-like (SKP1-CUL7-FBXW8) complex of E3 ligases.

To test whether levels of cyclin D1 protein are mainly regulated by the SCF or the SCF-like pathway, immunoblot analysis was performed 48 hours after depleting SKP1 expression with small interfering (si) RNA double-strand oligonucleotides in HCT 116 cells (FIG. 13, Panel B). siRNA for SKP1 significantly reduced SKP1 expression and resulted in accumulation of cyclin D1 without affecting the cell cycle profile. These experiments were repeated with SW480 colon cancer cells and T98G glioblastoma cells (data not shown) having similar results. These results demonstrate that stability of cyclin D1 is regulated through the SCF or the SCF-like pathway.

### Example 13

### The F-box Protein FBXW8 Specifically Associates with Cyclin D1 in a Thr286 Phosphorylation Dependent Manner

Candidate human F-box protein genes were tested to identify which one is the unique E3 ubiquitin ligase for cyclin D1. Substrate specificity of SCF complexes is determined through protein-protein interaction domains that are often tryptophan-aspartic acid (WD) 40 motifs or leucine-rich repeats (LRR) within F-box proteins (Cardozo, et al., 2004. Nat Rev Mol Cell Biol. 5, 739-51; Jin et al., 2004. Genes Dev. 18, 2573-80). The NCBI databases were searched for human F-box proteins with WD40 or LRR motifs. Approximately 70 potential genes containing F-box protein motifs were found. Among these, nine had WD40 repeat motifs and 17 had LRR motifs.

A reverse transcriptase-polymerase chain reaction (RT-PCR) was performed using total RNA from HEK 293, HCT 116 or WI-38 cells to obtain these 26 F-box protein genes. The full-length cDNAs that were retrieved were cloned into V5 or Flag epitope tag expression vectors. To address whether any of these 26 gene products could recognize cyclin D1, these Flag-tagged F-box proteins DNA plasmids were transiently transfected into T98G glioblastoma cells with or without N-terminal HA-tagged cyclin D1 and CDK4 expression vectors (FIG. 13, Panel C). After 24 hours, the cells were collected and an immunoprecipitated-immunoblotting analysis was performed. The samples were precipitated with an HA epitope tag antibody and subsequently stained with Flag (FBXW7 and FBXL5) or V5 (others), and cyclin D1 antibodies. FIG. 13, Panel C shows cyclin D1 associating with two F-box proteins. One F-box protein possessed WD40 motifs: FBXW8 (lane 7) and the other had LRR motifs: FBXL12 (lane 15).

Because F-box proteins substrates must be phosphorylated (Deshaies et al., 1999. Annu Rev Cell Dev Biol. 15, 435-67), FBXW8 and FBXL12 were tested to determine whether each one specifically recognizes cyclin D1 in a Thr286 phosphorylation-dependent manner (Cardozo et al., 2004. Nat Rev Mol Cell Biol. 5, 739-51). V5-tagged F-box proteins DNA plasmids together with cyclin D1 (wild type or the T286A mutant) and CDK4 expression vectors were transiently transfected into T98G glioblastoma cells.

Samples were precipitated with a HA epitope tag antibody and blotted with V5 and HA antibodies (FIG. 14, Panel A). FBXW8 was associated with both cyclin D1 wild type and the T286A mutant, but the majority was bound to wild type. In contrast, there was no significant difference between wild-type cyclin D1 and the mutant in association with FBXL12. These observations indicate that FBXW8, but not FBXL12, specifically recognizes cyclin D1 in a Thr286 phosphorylation-dependent manner.

To confirm this finding, an in vitro binding assay was performed (FIG. 14, Panel B; Carrono et al., 1999. Nat Cell Biol. 1, 193-9). ³⁵S-labeled in vitro translated FBXW8, FBXL12 or β-TRCP were incubated with rabbit reticulocyte cell extracts and beads coupled to either the Thr286 phosphorylated cyclin D1 peptide (Cyclin D1-P; lane 2, corresponding to the amino acids 282-291 of human cyclin D1) or unphosphorylated cyclin D1 peptide (lane 1, Cyclin D1). Lane 3 contains 50% input of each in in vitro-translated product. FBXW8 was specifically bound to Thr286 phosphorylated cyclin D1 peptide. In contrast, little association of FBXL12 with each peptide was observed, indicating that FBXL12 requires different sites from the C-terminus of cyclin D1 for association. Consistent with this finding, FBXL12 was not involved in polyubiquitination of cyclin D1 in vitro (FIG. 14, Panel C, lane 9). These results demonstrate that FBXW8 plays a role in cyclin D1 stability.

### Example 14

### FBXW8 Ubiquitinates Cyclin D1 in a Thr-286 Phosphorylation Dependent Manner

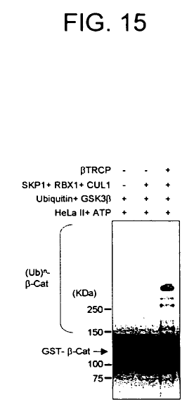

In order to determine whether in vitro ubiquitination of cyclin D1 requires FBXW8 (FIG. 14, Panel C), each in vitro-translated F-box protein was incubated with recombinant GST-cyclin D1 (CD1), Fraction II HeLa cell extracts with ATP, ubiquitin and ERK2, and in vitro-translated either SKP1, RBX1 and CUL1, or SKP1, RBX1 and CUL7 proteins, and then blotted with a cyclin D1 antibody. To confirm that the SCF complexes were assembled properly upon in vitro translation, an immmunoprecipitation was performed with each F-box protein in the ³⁵S-labeled in vitro translated samples (data not shown) and tested to determine whether the complexes containing β-TRCP were functional for polyubiquitination of β-catenin (FIG. 15). Ubiquitination of cyclin D1 was detected in the combinations of SKP1, CUL1, FBXW8, and RBX1 (lane 5), or SKP1, CUL7, FBXW8 and RBX1 (lane 6). However, polyubiquitinated-bands did not appear to be increased through other combinations. These results indicate that cyclin D1 ubiquitination involves FBXW8.

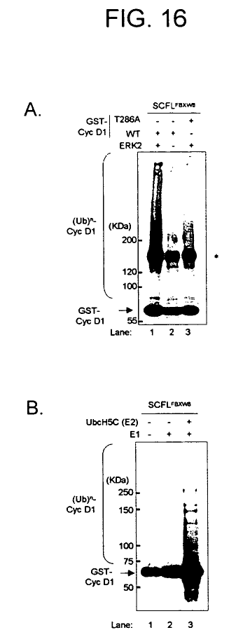

In vitro ubiquitination of cyclin D1 through the SCF-like (SCFL) complex FBXW8 (SKP1-CUL7-FBXW8-RBX1/SCFL^{FBXW8}) was investigated to determine whether it requires phosphorylation of cyclin D1 at Thr286 (FIG. 16, Panel A). Polyubiquitination through the SCFL^{FBXW8} was dramatically reduced by the depletion of ERK2 (lane 2). Furthermore, the polyubiquitination of cyclin D1 was largely prevented by the alanine-for-Thr286 substitution (T286A, lane 3), indicating that phosphorylation of cyclin D1 at Thr286 is necessary for ubiquitination by SCFL^{FBXW8}. These results confirm that FBXW8 specifically associates with cyclin D1 in a Thr286 phosphorylation-dependent manner.

Finally, polyubiquitination of cyclin D1 was reconstituted in vitro using purified E1 and E2 (FIG. 16, Panel B). The V5 immunoprecipitates containing SCFL^{FBXW8} exhibited significant E3 activities for polyubiquitination of cyclin D1 in the presence of both E1 and E2/UbcH5C. These results demonstrate that 1) cyclin D1 can be ubiquitinated by FBXW8 E3 ligase and 2) that this process is dependent on Thr286 phosphorylation of cyclin D1 by ERK/MAPK.

### Example 15

### Cyclin D1 Protein Levels are Regulated by FBXW8

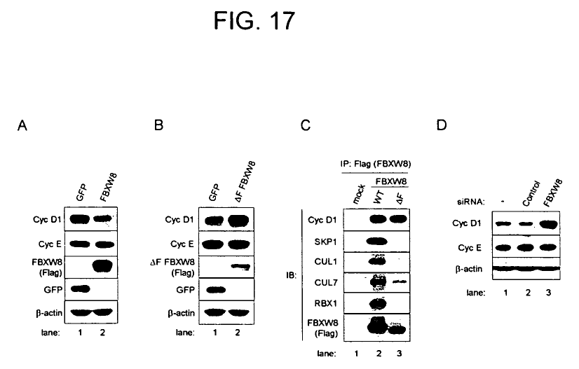

HCT 116 cells were infected with a retrovirus expressing the FBXW8 or a control retrovirus expressing GFP in order to determine whether ectopic expression of FBXW8 reduces levels of endogenous cyclin D1 in cultured cells (FIG. 17, Panel A). Overexpression of FBXW8 reduced endogenous expression of cyclin D1. However, ectopically expressed FBXW8 did not significantly change expression profiles of cyclin E. Similar profiles were obtained from SW480 colon cancers, U-2 OS osteosarcomas, and T98G glioblastomas (data not shown).

A dominant-negative form of FBXW8 was overexpressed to determine whether it causes accumulation of cyclin D1 protein in exponentially growing cultured cells. The F-box deletion ΔF) mutant form of FBXW8 serves as a dominant-negative because the mutant is able to bind to cyclin D1 but barely associates with SKP1, CUL1 and CUL7 (FIG. 17, Panel C, lane 3), and therefore does not bring cyclin D1 into the ubiquitin-proteasome pathway. HCT 116 cells were infected with the retrovirus expressing the ΔF FBXW8 mutant or a control retrovirus expressing GFP (FIG. 17, Panel B). Significant accumulation of cyclin D1 was observed following ΔF FBXW8 expression. In contrast, an ectopically expressed dominant-negative form of FBXW8 did not significantly change levels of another cell cycle regulator cyclin E. These experiments were repeated in SW480 colon cancer cells and T98G glioblastoma cells (data not shown) resulting in similar observations.

To confirm this finding, the depletion of endogenous FBXW8 expression by siRNA double-strand oligonucleotides was tested to determine whether it causes cyclin D1 protein to accumulate in HCT 116 cells (FIG. 17, Panel D). HCT 116 cells were treated with control or FBXW8 siRNA for 48 hours. Inhibition of FBXW8 was verified RT-PCR analysis. Approximately 95% inhibition of FBXW8 was observed compared to the control sample (data not shown). A significant accumulation of cyclin D1 was found in the sample treated with FBXW8 siRNA (lane 3) without affecting levels of cyclin E. These results demonstrate that cyclin D1 protein levels are regulated by FBXW8.

### Example 16

### The Stability of Cyclin D1 Protein is Regulated Through the Complexes Containing FBXW8

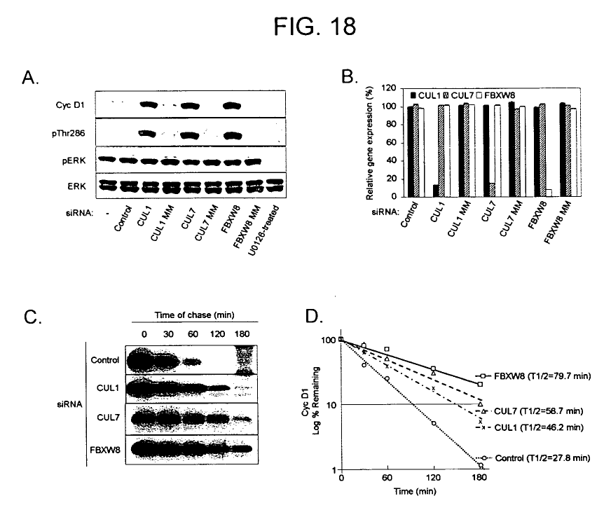

Expression of CUL1 or CUL7 was knocked down with siRNA double-strand oligonucleotides for 48 hours in HCT 116 cells (FIG. 18, Panels A and B). In parallel, RT-PCR analysis was performed to confirm that siRNA transfection was working efficiently (FIG. 18, Panel B). The siRNAs for CUL1, CUL7, or FBXW8 significantly reduced expression of CUL1, CUL7, or FBXW8 and resulted in accumulation of cyclin D1, which was mostly phosphorylated at Thr286 (FIG. 18, Panels A and B). The effect was achieved without affecting MAPK activities (pERK) in the first 48 hours of siRNA treatment (FIG. 18, Panel A). Comparable data were obtained from SW480 colon cancer cells, U-2 OS osteosarcoma cells, and T98 glioblastoma cells (data not shown).

To confirm that accumulation of cyclin D1 protein through depletion of FBXW8, CUL1, or CUL7 was due to an increase of cyclin D1 stability, a pulse-chase analysis was performed on metabolically labeled-cyclin D1 protein after depriving cell cultures of FBXW8, CUL1, or CUL7 from HCT 116 via siRNA double-strand oligonucleotides (FIG. 18, Panel C). Levels of metabolically labeled-cyclin D1 were estimated as described above (FIG. 18, Panel D). Reducing FBXW8, CUL7 or CUL1 led to stabilization of cyclin D1. The half-life of cyclin D1 was extended (T1/2=79.7, 58.7, or 46.2 min by FBXW8, CUL7, or CUL1 siRNA treatment, respectively) from control non-targeting siRNA-treated cells (T1/2=27.8 min). These results confirm that accumulation of cyclin D1 protein through depletion of FBXW8, CUL1, or CUL7 (FIG. 18, Panel A) was caused by the increase of cyclin D1 stability. These results demonstrate that cyclin D1 stability is regulated by complexes containing FBXW8, through the ubiquitin-proteasome pathway.

### Example 17

### FBXW8-mediated Cyclin D1 Degradation in the Cytoplasm is Required for Proliferation of Cancer Cells

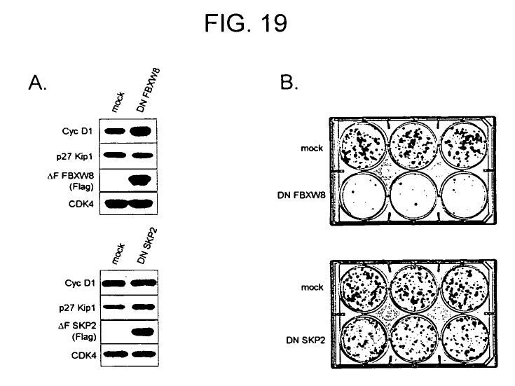

Cyclin D1 proteolysis was first inhibited in the cytoplasm through a dominant-negative (DN) form of FBXW8 (ΔF FBXW8) in order to determine whether degradation is necessary for proliferation in cancer cells through a colony-forming assay. Exponentially growing HCT 116 cells were infected with a retrovirus expressing a control empty vector (mock) or a DN FBXW8 or SKP2 (ΔF FBXW8 or ΔF SKP2; Carrano et al., 1999. Nat Cell Biol. 1, 193-9; Sutterluty et al., 1999. Nat Cell Biol. 1, 207-214). Infected cells were selected with G418 for 2 weeks. Western blot analysis was performed in mock-infected, DN FBXW8, and DN SKP2 cells to assess production of cyclin D1, p27 Kip1, CDK4 and either DN FBXW8 or DN SKP2 (where the latter were detected using an antibody that specifically binds the FLAG tag). Ectopic expression of ΔF FBXW8 reduced the number and size of colonies formed relative to the control. (FIG. 19, Panels A-B) However, ΔF SKP2 had little effect on cell growth because its major target p27 Kip1 (Nakayama et al., 2004) does not play any significant role in the growth control of HCT 116 cells (Tetsu et al., 2003. Cancer Cell. 3, 233-45). (FIGS. 19, Panels A-B) These results indicate that cyclin D1 proteolysis is crucial for proliferation of cancer cells.

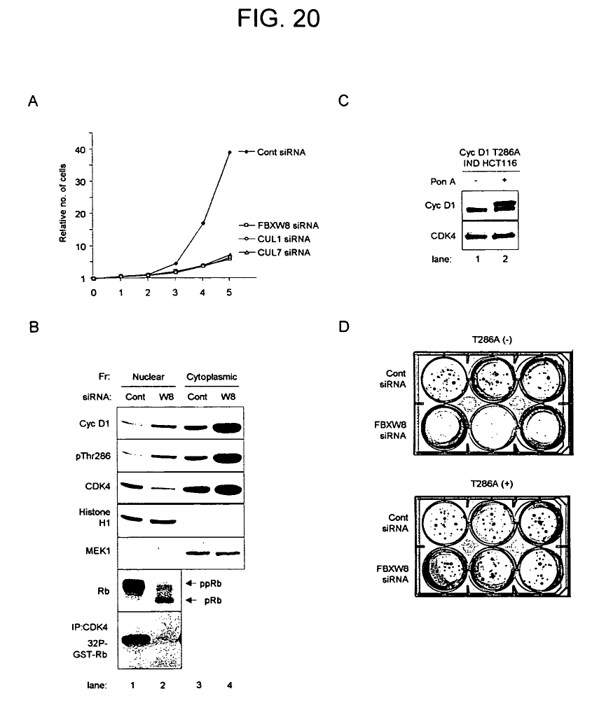

Cyclin D1 degradation was next inhibited by using siRNA to knock down E3 ligase components such as FBXW8, CUL1 or CUL7 in HCT 116 cells. The cell numbers were counted for five days (FIG. 20, Panel A) resulting in significantly reduced cell numbers in siRNA for FBXW8, CUL1, or CUL7. These results deomonstrate that the rapid turnover of cyclin D1 is required for proliferation of cancer cells.

The reduction of cell proliferation was tested through knockdown of FBXW8 expression is caused by accumulation of cyclin D1, and subsequent sequestration of CDK4 into the cytoplasm (FIG. 20, Panel B). HCT 116 cells were treated either with control (Cont) or FBXW8 (W8) siRNA for 72 hours. Inhibition of FBXW8 expression was verified by a RT-PCR. More than 95% inhibition of FBXW8 mRNA was observed compared to control samples. Nuclear and cytoplasmic proteins were fractioned. Panel B shows that depleting FBXW8 caused a significant accumulation of cyclin D1 protein in the cytoplasm, which was mostly phosphorylated at Thr286. This process resulted in relocalization of CDK4 from the nucleus to the cytoplasm. This caused dramatic reduction of the nuclear CDK4 kinase activities assessed by both phosphorylation status of Rb protein (pRb) and CDK4-associated GST-Rb in vitro kinase assay (FIG. 20, Panel B bottom). These observations show that inhibiting rapid turnover of cyclin D1 induced growth arrest in an Rb-dependent process.

The constitutive expression of the nuclear protein cyclin D1 T286A-CDK4 was examined to determine whether the complex could abrogate to block cell proliferation caused by siRNA against FBXW8 (FIG. 20, Panels C-D). Cyclin D1 mutant was tested because it is not only resistant to polyubiquitination but also prevents the nuclear export of cyclin D1 during S phase, resulting in its constitutive nuclear localization (Alt et al., 2000. Genes Dev. 14, 3102-14). Importantly, this mutant is functional; ectopically expressed T286A assembled with CDK4 in cultured cells and showed similar levels of kinase activities to wild type cyclin D1 as others demonstrated previously (data not shown; Cheng et al., 1999. EMBO J. 18, 1571-83).

A cyclin D1 ecdysone-inducible (IND) system in HCT 116 cells was generated. Pon A induced ectopic expression of HA-tagged T286A in physiological levels (FIG. 20, Panel C). A colony formation assay (FIG. 20, Panel D) was then performed. One hundred single cells from T286A IND HCT 116 were cultured in the presence (+) or absence (−) of Pon A, and control (Cont) or FBXW8 siRNA. Cells were cultured for 2 weeks, and stained with crystal violet. FIG. 20, Panel D shows that ectopically expressed physiological levels of nuclear protein cyclin D1 T286A dramatically rescued cells from growth arrest. These results demonstrate that FBXWS-mediated cyclin D1 degradation is essential for proliferation of cancer cells.

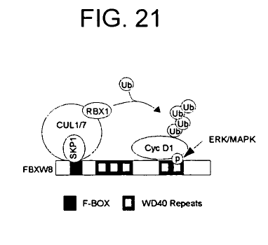

FIG. 21 is a schematic showing a model of ubiquitination of cyclin D1 through the complex containing FBXW8 provides a FBXW8 recognizes cyclin D1 through a WD40 repeat motif in an ERK/MAPK-mediated Thr286 phosphorylation-dependent manner. SKP1 interacts with FBXW8 together with CUL1 or CUL7 via a domain called F-box in the N-terminus. CUL1 or CUL7 recruits RBX1, which in turn conscripts an ubiquitin-conjugating enzyme E2 to add a multiubiquitin chain to cyclin D1.

### Example 18

### Production of an Antibody that Specifically Binds Phosphorylated Cyclin D1

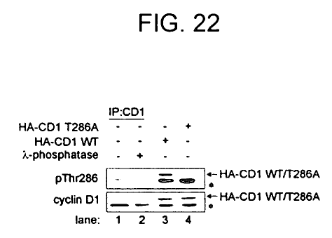

A phosphorylation-specific polyclonal antibody was established in order to detect Thr286 phosphorylation (FIG. 22). The antibody was used to detect cyclin D1 in stable SW480 cells expressing HA-tagged wildtype (WT) cyclin D1 or HA-tagged cyclin D1 T286A (FIG. 6, Panel A). The anti-phosphorylated cyclin D1 antibody detected WT cyclin D1, but not cyclin D1 T286A protein (FIG. 22, lanes 3 and 4).

That which is claimed is:

1. A method for controlling cell proliferation comprising:
contacting a cell with an agent that modulates activity of a FBXW8 polypeptide, thereby controlling cell proliferation.

2. The method of claim 1, wherein the agent modulates activity of the FBXW8 polypeptide by:
modulating transcription of a nucleic acid encoding the FBXW8 polypeptide,
modulating translation of a nucleic acid encoding the FBXW8 polypeptide,
modulating activation of a n E3 complex comprising the FBXW8 polypeptide,
modulating degradation of the FBXW8 polypeptide, or
modulating interaction of FBXW8 with cyclin D1 polypeptide.

3. A method for decreasing cell proliferation comprising:
contacting a cell with an agent, wherein the agent decreases activity of a FBXW8 polypeptide, thereby decreasing cell proliferation.

4. The method of claim 3, wherein the agent modulates activity of the FBXW8 polypeptide by:
modulating transcription of a nucleic acid encoding the FBXW8 polypeptide,
modulating translation of a nucleic acid encoding the FBXW8 polypeptide,
modulating activation of a n E3 complex comprising the FBXW8 polypeptide,
modulating degradation of the FBXW8 polypeptide, or
modulating interaction of FBXW8 with cyclin D1 polypeptide.

5. The method of claim 3, wherein cell proliferation is associated with cancer or tumor growth.

6. The method of claim 3, wherein the agent is a MAP kinase inhibitor, a Raf inhibitor, or an MEK inhibitor.

7. A method of screening a test agent for activity in modulating cell proliferation, the method comprising:
contacting a FBXW8 polypeptide and a phosphorylated cyclin D1 polypeptide with a test agent, said contacting being under conditions suitable for interaction of a FBXW8 polypeptide and a phosphorylated cyclin D1 polypeptide to provide for ubiquitination of the phosphorylated cyclin D1 polypeptide by the FBXW8 polypeptide;
detecting the presence or absence of an effect of the test agent upon interaction between the FBXW8 polypeptide and the cyclin D1 polypeptide;
wherein an effect of the test agent upon said interaction in the presence of the test agent as compared to the absence of the test agent indicates the test agent is capable of modulating cell proliferation.

8. The method of claim 7, wherein said detecting is by detecting an effect of the test agent on binding of the FBXW8 polypeptide to the phosphorylated cyclin D1 polypeptide in an in vitro assay.

9. The method of claim 7, wherein said detecting is by detecting an effect of the test agent on ubiquitination of phosphorylated cyclin D1 polypeptide by the FBXW8 polypeptide in an in vitro assay.

10. The method of claim 7, wherein said contacting is in the presence of a detectably labeled ubiquitin molecule, and said detecting the effect of the test agent on levels of detectably labeled, ubiquitinated cyclin D1 polypeptide .

11. The method of claim 7, wherein said detecting is by detecting an effect of the test agent on binding of the FBXW8 polypeptide to the phosphorylated cyclin D1 polypeptide in a cell-based assay.

12. The method of claim 7, wherein said detecting is by detecting an effect of the test agent on ubiquitination of phosphorylated cyclin D1 polypeptide by the FBXW8 polypeptide in a cell-based assay.

13. The method of claim 7, wherein said detecting is by detecting an effect of the test agent on total phosphorylated cyclin D1 polypeptide levels in a cell, and wherein said effect is specific for interaction of the FBXW8 polypeptide and the cyclin D1 polypeptide.

14. The method of claim 7, wherein said detecting is by detecting an effect of the test agent on total levels of cyclin D1 polypeptide in a cell, and wherein said effect is specific for interaction of the FBXW8 polypeptide and the cyclin D1 polypeptide.

15. The method of claim 7, wherein said detecting is by detecting an effect of the test agent on total levels of ubiquitinated cyclin D1 in a cell.

16. The method of claim 15, wherein the cell comprises a detectably labeled ubiquitin molecule, and said detecting the effect of the test agent on levels of detectably labeled, ubiquitinated cyclin D1 in the cell.

17. The method of claim 7, wherein at least one of the FBXW8 polypeptide and the cyclin D1 polypeptide are expressed from a recombinant nucleic acid construct in a cell.

18. The method of claim 17, wherein at least one of the FBXW8 polypeptide and cyclin D1 polypeptide are provided as a fusion protein comprising a detectable label.

19. The method of claim 18, wherein the detectable label is an immunodetectable label, an enzymatic polypeptide, or a fluorescent polypeptide.

20. The method of claim 19, wherein the immunodetectable label comprises a FLAG epitope.

21. The method of claim 19, wherein the enzymatic polypeptide is glutathione-S-transferase.

22. The method of claim 19, wherein the fluorescent polypeptide is a green fluorescent polypeptide.

23. A method of screening a test agent for activity in modulating cell proliferation, the method comprising:
contacting a MAPK polypeptide and a cyclin D1 polypeptide with a test agent, said contacting being under conditions suitable for interaction of a MAPK polypeptide and cyclin D1 polypeptide to provide for phosphorylation of the cyclin D1 polypeptide by the MAPK polypeptide;
detecting the presence or absence of an effect of the test agent upon interaction between the MAPK polypeptide and the cyclin D1 polypeptide;
wherein an effect of the test agent upon said interaction in the presence of the test agent as compared to the absence of the test agent indicates the test agent is capable of modulating cell proliferation.

24. The method of claim 23, wherein said detecting is by detecting an effect of the test agent on binding of the MAPK polypeptide to the cyclin D1 polypeptide in an in vitro assay.

25. The method of claim 23, wherein said detecting is by detecting an effect of the test agent on phosphorylation cyclin D1 by the MAPK polypeptide in an in vitro assay.

26. The method of claim 23, wherein said detecting is by detecting an effect of the test agent on binding of the MAPK polypeptide to the cyclin D1 polypeptide in a cell-based assay.

27. The method of claim 23, wherein said detecting is by detecting an effect of the test agent on phosphorylation of the cyclin D1 polypeptide by the MAPK polypeptide in a cell-based assay.

28. The method of claim 23, wherein said detecting is by detecting an effect of the test agent on total levels of phosphorylated cyclin D1 in a cell, and wherein said effect is specific for interaction of a MAPK polypeptide and a cyclin D1 polypeptide.

29. The method of claim 23, wherein said detecting is by detecting an effect of the test agent on total levels of cyclin D1 in a cell, and wherein said effect is specific for interaction of a MAPK polypeptide and a cyclin D1 polypeptide.

30. The method of claim 23, wherein said detecting is by detecting an effect of the test agent on total levels of ubiquitinated cyclin D1 in a cell, and wherein said effect is specific for interaction of a MAPK polypeptide and a cyclin D1 polypeptide.

31. The method of claim 23, wherein at least one of the MAPK polypeptide and the cyclin D1 polypeptide are expressed from a recombinant nucleic acid construct in a cell.

32. The method of claim 31, wherein at least one of the MAPK polypeptide and cyclin D1 polypeptide are provided as a fusion protein comprising a detectable label.

33. The method of claim 32, wherein the detectable label is an immunodetectable label, an enzymatic polypeptide, or a fluorescent polypeptide.

34. The method of claim 33, wherein the immunodetectable label comprises a FLAG epitope.

35. The method of claim 33, wherein the enzymatic polypeptide is glutathione-S-transferase.

36. The method of claim 33, wherein the fluorescent polypeptide is a green fluorescent polypeptide.

37. An isolated polypeptide complex comprising:
a FBXW8 polypeptide;
a Cullin polypeptide, wherein the Cullin polypeptide is a CUL1 polypeptide or a CUL7 polypeptide;
a SKP1 polypeptide; and
a phosphorylated cyclin D1 polypeptide;
wherein the complex is capable of binding a phosphorylated cyclin D1 polypeptide.

38. The isolated polypeptide complex of claim 37, wherein at least one polypeptide of the complex is detectably labeled.

39. A reaction mixture comprising:
an isolated cyclin D1; and
an isolated MAPK polypeptide.

40. The reaction mixture of claim 39, further comprising a source of phosphate for phosphorylation of cyclin D1 by MAPK.

41. A reaction mixture comprising:
an isolated complex comprising
an isolated FBXW8 polypeptide,
a Cullin polypeptide, wherein the Cullin polypeptide is a CUL1 polypeptide or
a CUL7 polypeptide, and
a SKP1 polypeptide; and
an isolated phosphorylated cyclin D1 polypeptide.

42. A method for inhibiting cell proliferation comprising:
contacting a cell with an effective amount of a small interfering nucleic acid (siNA) for at least one of an FBXW8-encoding nucleic acid, a CUL1-encoding nucleic acid, or a CUL7-encoding nucleic acid;
wherein said contacting provides for inhibition of proliferation of the cell.

43. The method of claim 42, wherein the cell is a cancerous cell.

44. The method of claim 42, wherein said contacting is effective to inhibit growth of a tumor.

45. A composition comprising:
an isolated small interfering nucleic acid (siNA), wherein the siNA comprises a sequence effective to inhibit transcription or translation of an FBXW8-encoding nucleic acid, a CUL1-encoding nucleic acid, or a CUL7-encoding nucleic acid; and
a pharmaceutically acceptable carrier.
